SEQUENCE LISTING (35 sequences)

SEQ ID NO: 1 - DNA, 25 bp, Artificial Sequence
  source: /note=“Description of Artificial Sequence Synthetic oligonucleotide”
gttcttaaggcacaggaactgggac

SEQ ID NO: 2 - DNA, 25 bp, Artificial Sequence
  source: /note=“Description of Artificial Sequence Synthetic oligonucleotide”
gaagttaaccctgtcgttctgcgac

SEQ ID NO: 3 - DNA, 25 bp, Artificial Sequence
  source: /note=“Description of Artificial Sequence Synthetic oligonucleotide”
gttctatagggtctgcttgtcgctc

SEQ ID NO: 4 - DNA, 34 bp, Artificial Sequence
  source: /note=“Description of Artificial Sequence Synthetic probe”
gccagtcccagttcctgtgccttaagaacctcgc

SEQ ID NO: 5 - DNA, 34 bp, Artificial Sequence
  source: /note=“Description of Artificial Sequence Synthetic probe”
gcgagtcgcagaacgacagggttaacttcctcgc

SEQ ID NO: 6 - DNA, 5997 bp, Homo sapiens
atggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataa

SEQ ID NO: 7 - DNA, 6018 bp, Homo sapiens
atggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcatatgtgacagagtttgtggacctgggcaatgtctcagcgttgagaacattcagagttctccgagcattgaaaacaatttcagtcattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaa

SEQ ID NO: 8 - DNA, 6003 bp, Homo sapiens
atggcacaggcactgttggtacccccaggacctgaaagcttccgcctttttactagagaatctcttgctgctatcgaaaaacgtgctgcagaagagaaagccaagaagcccaaaaaggaacaagataatgatgatgagaacaaaccaaagccaaatagtgacttggaagctggaaagaaccttccatttatttatggagacattcctccagagatggtgtcagagcccctggaggacctggatccctactatatcaataagaaaacttttatagtaatgaataaaggaaaggcaattttccgattcagtgccacctctgccttgtatattttaactccactaaaccctgttaggaaaattgctatcaagattttggtacattctttattcagcatgcttatcatgtgcactattttgaccaactgtgtatttatgaccttgagcaaccctcctgactggacaaagaatgtagagtacacattcactggaatctatacctttgagtcacttataaaaatcttggcaagagggttttgcttagaagattttacgtttcttcgtgatccatggaactggctggatttcagtgtcattgtgatggcatatgtgacagagtttgtggacctgggcaatgtctcagcgttgagaacattcagagttctccgagcactgaaaacaatttcagtcattccaggtttaaagaccattgtgggggccctgatccagtcggtaaagaagctttctgatgtgatgatcctgactgtgttctgtctgagcgtgtttgctctcattgggctgcagctgttcatgggcaatctgaggaataaatgtttgcagtggcccccaagcgattctgcttttgaaaccaacaccacttcctactttaatggcacaatggattcaaatgggacatttgttaatgtaacaatgagcacatttaactggaaggattacattggagatgacagtcacttttatgttttggatgggcaaaaagaccctttactctgtggaaatggctcagatgcaggccagtgtccagaaggatacatctgtgtgaaggctggtcgaaaccccaactatggctacacaagctttgacacctttagctgggctttcctgtctctatttcgactcatgactcaagactactgggaaaatctttaccagttgacattacgtgctgctgggaaaacatacatgatattttttgtcctggtcattttcttgggctcattttatttggtgaatttgatcctggctgtggtggccatggcctatgaggagcagaatcaggccaccttggaagaagcagaacaaaaagaggccgaatttcagcagatgctcgaacagcttaaaaagcaacaggaagaagctcaggcagttgcggcagcatcagctgcttcaagagatttcagtggaataggtgggttaggagagctgttggaaagttcttcagaagcatcaaagttgagttccaaaagtgctaaagaatggaggaaccgaaggaagaaaagaagacagagagagcaccttgaaggaaacaacaaaggagagagagacagctttcccaaatccgaatctgaagacagcgtcaaaagaagcagcttccttttctccatggatggaaacagactgaccagtgacaaaaaattctgctcccctcatcagtctctcttgagtatccgtggctccctgttttccccaagacgcaatagcaaaacaagcattttcagtttcagaggtcgggcaaaggatgttggatctgaaaatgactttgctgatgatgaacacagcacatttgaagacagcgaaagcaggagagactcactgtttgtgccgcacagacatggagagcgacgcaacagtaacgttagtcaggccagtatgtcatccaggatggtgccagggcttccagcaaatgggaagatgcacagcactgtggattgcaatggtgtggtttccttggtgggtggaccttcagctctaacgtcacctactggacaacttcccccagagggcaccaccacagaaacggaagtcagaaagagaaggttaagctcttaccagatttcaatggagatgctggaggattcctctggaaggcaaagagccgtgagcatagccagcattctgaccaacacaatggaagaacttgaagaatctagacagaaatgtccgccatgctggtatagatttgccaatgtgttcttgatctgggactgctgtgatgcatggttaaaagtaaaacatcttgtgaatttaattgttatggatccatttgttgatcttgccatcactatttgcattgtcttaaataccctctttatggccatggagcactaccccatgactgagcaattcagtagtgtgttgactgtaggaaacctggtctttactgggattttcacagcagaaatggttctcaagatcattgccatggatccttattactatttccaagaaggctggaatatctttgatggaattattgtcagcctcagtttaatggagcttggtctgtcaaatgtggagggattgtctgtactgcgatcattcagactgcttagagttttcaagttggcaaaatcctggcccacactaaatatgctaattaagatcattggcaattctgtgggggctctaggaaacctcaccttggtgttggccatcatcgtcttcatttttgctgtggtcggcatgcagctctttggtaagagctacaaagaatgtgtctgcaagatcaatgatgactgtacgctcccacggtggcacatgaacgacttcttccactccttcctgattgtgttccgcgtgctgtgtggagagtggatagagaccatgtgggactgtatggaggtcgctggccaaaccatgtgccttattgttttcatgttggtcatggtcattggaaaccttgtggttctgaacctctttctggccttattgttgagttcatttagctcagacaaccttgctgctactgatgatgacaatgaaatgaataatctgcagattgcagtaggaagaatgcaaaagggaattgattatgtgaaaaataagatgcgggagtgtttccaaaaagccttttttagaaagccaaaagttatagaaatccatgaaggcaataagatagacagctgcatgtccaataatactggaattgaaataagcaaagagcttaattatcttagagatgggaatggaaccaccagtggtgtaggtactggaagcagtgttgaaaaatacgtaatcgatgaaaatgattatatgtcattcataaacaaccccagcctcaccgtcacagtgccaattgctgttggagagtctgactttgaaaacttaaatactgaagagttcagcagtgagtcagaactagaagaaagcaaagagaaattaaatgcaaccagctcatctgaaggaagcacagttgatgttgttctaccccgagaaggtgaacaagctgaaactgaacccgaagaagaccttaaaccggaagcttgttttactgaaggatgtattaaaaagtttccattctgtcaagtaagtacagaagaaggcaaagggaagatctggtggaatcttcgaaaaacctgctacagtattgttgagcacaactggtttgagactttcattgtgttcatgatccttctcagtagtggtgcattggcctttgaagatatatacattgaacagcgaaagactatcaaaaccatgctagaatatgctgacaaagtctttacctatatattcattctggaaatgcttctcaaatgggttgcttatggatttcaaacatatttcactaatgcctggtgctggctagatttcttgatcgttgatgtttctttggttagcctggtagccaatgctcttggctactcagaactcggtgccatcaaatcattacggacattaagagctttaagacctctaagagccttatcccggtttgaaggcatgagggtggttgtgaatgctcttgttggagcaattccctctatcatgaatgtgctgttggtctgtctcatcttctggttgatctttagcatcatgggtgtgaatttgtttgctggcaagttctaccactgtgttaacatgacaacgggtaacatgtttgacattagtgatgttaacaatttgagtgactgtcaggctcttggcaagcaagctcggtggaaaaacgtgaaagtaaactttgataatgttggcgctggctatcttgcactgcttcaagtggccacatttaaaggctggatggatattatgtatgcagctgttgattcacgagatgttaaacttcagcctgtatatgaagaaaatctgtacatgtatttatactttgtcatctttatcatctttgggtcattcttcactctgaatctattcattggtgtcatcatagataacttcaaccagcagaaaaagaagtttggaggtcaagacatctttatgacagaggaacagaaaaaatattacaatgcaatgaagaaacttggatccaagaaacctcagaaacccatacctcgcccagcaaacaaattccaaggaatggtctttgattttgtaaccagacaagtctttgatatcagcatcatgatcctcatctgcctcaacatggtcaccatgatggtggaaacggatgaccagggcaaatacatgaccctagttttgtcccggatcaacctagtgttcattgttctgttcactggagaatttgtgctgaagctcgtctccctcagacactactacttcactataggctggaacatctttgactttgtggtggtgattctctccattgtaggtatgtttctggctgagatgatagaaaagtattttgtgtcccctaccttgttccgagtgatccgtcttgccaggattggccgaatcctacgtctgatcaaaggagcaaaggggatccgcacgctgctctttgctttgatgatgtcccttcctgcgttgtttaacatcggcctcctgctcttcctggtcatgtttatctatgccatctttgggatgtccaactttgcctatgttaaaaaggaagctggaattgatgacatgttcaactttgagacctttggcaacagcatgatctgcttgttccaaattacaacctctgctggctgggatggattgctagcacctattcttaatagtgcaccacccgactgtgaccctgacacaattcaccctggcagctcagttaagggagactgtgggaacccatctgttgggattttcttttttgtcagttacatcatcatatccttcctggttgtggtgaacatgtacatcgcggtcatcctggagaacttcagtgttgctactgaagaaagtgcagagcccctgagtgaggatgactttgagatgttctatgaggtttgggaaaagtttgatcccgatgcgacccagtttatagagttctctaaactctctgattttgcagctgccctggatcctcctcttctcatagcaaaacccaacaaagtccagcttattgccatggatctgcccatggtcagtggtgaccggatccactgtcttgatattttatttgcctttacaaagcgtgttttgggtgagagtggagagatggatgcccttcgaatacagatggaagacaggtttatggcatcaaacccctccaaagtctcttatgagcctattacaaccactttgaaacgtaaacaagaggaggtgtctgccgctatcattcagcgtaatttcagatgttatcttttaaagcaaaggttaaaaaatatatcaagtaactataacaaagaggcaattaaagggaggattgacttacctataaaacaagacatgattattgacaaactaaatgggaactccactccagaaaaaacagatgggagttcctctaccacctctcctccttcctatgatagtgtaacaaaaccagacaaggaaaagtttgagaaagacaaaccagaaaaagaaagcaaaggaaaagaggtcagagaaaatcaaaagtaa

SEQ ID NO: 9 - DNA, 5511 bp, Homo sapiens
atggccagaccatctctgtgcaccctggtgcctctgggccctgagtgcttgcgccccttcacccgggagtcactggcagccatagaacagcgggcggtggaggaggaggcccggctgcagcggaataagcagatggagattgaggagcccgaacggaagccacgaagtgacttggaggctggcaagaacctacccatgatctacggagaccccccgccggaggtcatcggcatccccctggaggacctggatccctactacagcaataagaagaccttcatcgtactcaacaagggcaaggccatcttccgcttctccgccacacctgctctctacctgctgagccccttcagcgtagtcaggcgcggggccatcaaggtgctcatccatgcgctgttcagcatgttcatcatgatcaccatcttgaccaactgcgtattcatgaccatgagtgacccgcctccctggtccaagaatgtggagtacaccttcacagggatctacacctttgagtccctcatcaagatactggcccgaggcttctgtgtcgacgacttcacattcctccgggacccctggaactggctggacttcagtgtcatcatgatggcgtacctgacagagtttgtggacttgggcaacatctcagccctgaggaccttccgggtgctgcgggccctcaaaaccatcacggtcatcccagggctgaagacgatcgtgggggccctgatccagtcggtgaaaaagctgtcggatgtgatgatcctcactgtcttctgcctgagcgtctttgcgctggtaggactgcagctcttcatgggaaacctgaggcagaagtgtgtgcgctggcccccgccgttcaacgacaccaacaccacgtggtacagcaatgacacgtggtacggcaatgacacatggtatggcaatgagatgtggtacggcaatgactcatggtatgccaacgacacgtggaacagccatgcaagctgggccaccaacgatacctttgattgggacgcctacatcagtgatgaagggaacttctacttcctggagggctccaacgatgccctgctctgtgggaacagcagtgatgctgggcactgccctgagggttatgagtgcatcaagaccgggcggaaccccaactatggctacaccagctatgacaccttcagctgggccttcttggctctcttccgcctcatgacacaggactattgggagaacctcttccagctgacccttcgagcagctggcaagacctacatgatcttcttcgtggtcatcatcttcctgggctctttctacctcatcaatctgatcctggccgtggtggccatggcatatgccgagcagaatgaggccaccctggccgaggataaggagaaagaggaggagtttcagcagatgcttgagaagttcaaaaagcaccaggaggagctggagaaggccaaggccgcccaagctctggaaggtggggaggcagatggggacccagcccatggcaaagactgcaatggcagcctggacacatcgcaaggggagaagggagccccgaggcagagcagcagcggagacagcggcatctccgacgccatggaagaactggaagaggcccaccaaaagtgcccaccatggtggtacaagtgcgcccacaaagtgctcatatggaactgctgcgccccgtggctgaagttcaagaacatcatccacctgatcgtcatggacccgttcgtggacctgggcatcaccatctgcatcgtgctcaacaccctcttcatggccatggaacattaccccatgacggagcactttgacaacgtgctcactgtgggcaacctggtcttcacaggcatcttcacagcagagatggttctgaagctgattgccatggacccctacgagtatttccagcagggttggaatatcttcgacagcatcatcgtcaccctcagcctggtagagctaggcctggccaacgtacagggactgtctgtgctacgctccttccgtctgctgcgggtcttcaagctggccaagtcgtggccaacgctgaacatgctcatcaagatcattggcaattcagtgggggcgctgggtaacctgacgctggtgctggctatcatcgtgttcatcttcgccgtggtgggcatgcagctgtttggcaagagctacaaggagtgcgtgtgcaagattgccttggactgcaacctgccgcgctggcacatgcatgatttcttccactccttcctcatcgtcttccgcatcctgtgcggggagtggatcgagaccatgtgggactgcatggaggtggccggccaagccatgtgcctcaccgtcttcctcatggtcatggtcatcggcaatcttgtggtcctgaacctgttcctggctctgctgctgagctccttcagcgccgacagtctggcagcctcggatgaggatggcgagatgaacaacctgcagattgccatcgggcgcatcaagttgggcatcggctttgccaaggccttcctcctggggctgctgcatggcaagatcctgagccccaaggacatcatgctcagcctcggggaggctgacggggccggggaggctggagaggcgggggagactgcccccgaggatgagaagaaggagccgcccgaggaggacctgaagaaggacaatcacatcctgaaccacatgggcctggctgacggccccccatccagcctcgagctggaccaccttaacttcatcaacaacccctacctgaccatacaggtgcccatcgcctccgaggagtccgacctggagatgcccaccgaggaggaaaccgacactttctcagagcctgaggatagcaagaagccgccgcagcctctctatgatgggaactcgtccgtctgcagcacagctgactacaagccccccgaggaggaccctgaggagcaggcagaggagaaccccgagggggagcagcctgaggagtgcttcactgaggcctgcgtgcagcgctggccctgcctctacgtggacatctcccagggccgtgggaagaagtggtggactctgcgcagggcctgcttcaagattgtcgagcacaactggttcgagaccttcattgtcttcatgatcctgctcagcagtggggctctggccttcgaggacatctacattgagcagcggcgagtcattcgcaccatcctagaatatgccgacaaggtcttcacctacatcttcatcatggagatgctgctcaaatgggtggcctacggctttaaggtgtacttcaccaacgcctggtgctggctcgacttcctcatcgtggatgtctccatcatcagcttggtggccaactggctgggctactcggagctgggacccatcaaatccctgcggacactgcgggccctgcgtcccctgagggcactgtcccgattcgagggcatgagggtggtggtgaacgccctcctaggcgccatcccctccatcatgaatgtgctgcttgtctgcctcatcttctggctgatcttcagcatcatgggtgtcaacctgtttgccggcaagttctactactgcatcaacaccaccacctctgagaggttcgacatctccgaggtcaacaacaagtctgagtgcgagagcctcatgcacacaggccaggtccgctggctcaatgtcaaggtcaactacgacaacgtgggtctgggctacctctccctcctgcaggtggccaccttcaagggttggatggacatcatgtatgcagccgtggactcccgggagaaggaggagcagccgcagtacgaggtgaacctctacatgtacctctactttgtcatcttcatcatctttggctccttcttcaccctcaacctcttcattggcgtcatcattgacaacttcaaccagcagaagaagaagttaggggggaaagacatctttatgacggaggaacagaagaaatactataacgccatgaagaagcttggctccaagaagcctcagaagccaattccccggccccagaacaagatccagggcatggtgtatgacctcgtgacgaagcaggccttcgacatcaccatcatgatcctcatctgcctcaacatggtcaccatgatggtggagacagacaaccagagccagctcaaggtggacatcctgtacaacatcaacatgatcttcatcatcatcttcacaggggagtgcgtgctcaagatgctcgccctgcgccagtactacttcaccgttggctggaacatctttgacttcgtggtcgtcatcctgtccattgtgggccttgccctctctgacctgatccagaagtacttcgtgtcacccacgctgttccgtgtgatccgcctggcgcggattgggcgtgtcctgcggctgatccgcggggccaagggcatccggacgctgctgttcgccctcatgatgtcgctgcctgccctcttcaacatcggcctcctcctcttcctggtcatgttcatctactccatcttcggcatgtccaactttgcctacgtcaagaaggagtcgggcatcgatgatatgttcaacttcgagaccttcggcaacagcatcatctgcctgttcgagatcaccacgtcggccggctgggacgggctcctcaaccccatcctcaacagcgggcccccagactgtgaccccaacctggagaacccgggcaccagtgtcaagggtgactgcggcaacccctccatcggcatctgcttcttctgcagctatatcatcatctccttcctcatcgtggtcaacatgtacatcgccatcatcctggagaacttcaatgtggccacagaggagagcagcgagccccttggtgaagatgactttgagatgttctacgagacatgggagaagttcgaccccgacgccacccagttcatcgcctacagccgcctctcagacttcgtggacaccctgcaggaaccgctgaggattgccaagcccaacaagatcaagctcatcacactggacttgcccatggtgccaggggacaagatccactgcctggacatcctctttgccctgaccaaagaggtcctgggtgactctggggaaatggacgccctcaagcagaccatggaggagaagttcatggcagccaacccctccaaggtgtcctacgagcccatcaccaccaccctcaagaggaagcacgaggaggtgtgcgccatcaagatccagagggcctaccgccggcacctgctacagcgctccatgaagcaggcatcctacatgtaccgccacagccacgacggcagcggggatgacgcccctgagaaggaggggctgcttgccaacaccatgagcaagatgtatggccacgagaatgggaacagcagctcgccaagcccggaggagaagggcgaggcaggggacgccggacccactatggggctgatgcccatcagcccctcagacactgcctggcctcccgcccctcccccagggcagactgtgcgcccaggtgtcaaggagtctcttgtctag

SEQ ID NO: 10 - DNA, 6051 bp, Homo sapiens
atggcaaacttcctattacctcggggcaccagcagcttccgcaggttcacacgggagtccctggcagccatcgagaagcgcatggcagagaagcaagcccgcggctcaaccaccttgcaggagagccgagaggggctgcccgaggaggaggctccccggccccagctggacctgcaggcctccaaaaagctgccagatctctatggcaatccaccccaagagctcatcggagagcccctggaggacctggaccccttctatagcacccaaaagactttcatcgtactgaataaaggcaagaccatcttccggttcagtgccaccaacgccttgtatgtcctcagtcccttccaccccatccggagagcggctgtgaagattctggttcactcgctcttcaacatgctcatcatgtgcaccatcctcaccaactgcgtgttcatggcccagcacgaccctccaccctggaccaagtatgtcgagtacaccttcaccgccatttacacctttgagtctctggtcaagattctggctcgaggcttctgcctgcacgcgttcactttccttcgggacccatggaactggctggactttagtgtgattatcatggcatacacaactgaatttgtggacctgggcaatgtctcagccttacgcaccttccgagtcctccgggccctgaaaactatatcagtcatttcagggctgaagaccatcgtgggggccctgatccagtctgtgaagaagctggctgatgtgatggtcctcacagtcttctgcctcagcgtctttgccctcatcggcctgcagctcttcatgggcaacctaaggcacaagtgcgtgcgcaacttcacagcgctcaacggcaccaacggctccgtggaggccgacggcttggtctgggaatccctggacctttacctcagtgatccagaaaattacctgctcaagaacggcacctctgatgtgttactgtgtgggaacagctctgacgctgggacatgtccggagggctaccggtgcctaaaggcaggcgagaaccccgaccacggctacaccagcttcgattcctttgcctgggcctttcttgcactcttccgcctgatgacgcaggactgctgggagcgcctctatcagcagaccctcaggtccgcagggaagatctacatgatcttcttcatgcttgtcatcttcctggggtccttctacctggtgaacctgatcctggccgtggtcgcaatggcctatgaggagcaaaaccaagccaccatcgctgagaccgaggagaaggaaaagcgcttccaggaggccatggaaatgctcaagaaagaacacgaggccctcaccatcaggggtgtggataccgtgtcccgtagctccttggagatgtcccctttggccccagtaaacagccatgagagaagaagcaagaggagaaaacggatgtcttcaggaactgaggagtgtggggaggacaggctccccaagtctgactcagaagatggtcccagagcaatgaatcatctcagcctcacccgtggcctcagcaggacttctatgaagccacgttccagccgcgggagcattttcacctttcgcaggcgagacctgggttctgaagcagattttgcagatgatgaaaacagcacagcgggggagagcgagagccaccacacatcactgctggtgccctggcccctgcgccggaccagtgcccagggacagcccagtcccggaacctcggctcctggccacgccctccatggcaaaaagaacagcactgtggactgcaatggggtggtctcattactgggggcaggcgacccagaggccacatccccaggaagccacctcctccgccctgtgatgctagagcacccgccagacacgaccacgccatcggaggagccaggcgggccccagatgctgacctcccaggctccgtgtgtagatggcttcgaggagccaggagcacggcagcgggccctcagcgcagtcagcgtcctcaccagcgcactggaagagttagaggagtctcgccacaagtgtccaccatgctggaaccgtctcgcccagcgctacctgatctgggagtgctgcccgctgtggatgtccatcaagcagggagtgaagttggtggtcatggacccgtttactgacctcaccatcactatgtgcatcgtactcaacacactcttcatggcgctggagcactacaacatgacaagtgaattcgaggagatgctgcaggtcggaaacctggtcttcacagggattttcacagcagagatgaccttcaagatcattgccctcgacccctactactacttccaacagggctggaacatcttcgacagcatcatcgtcatccttagcctcatggagctgggcctgtcccgcatgagcaacttgtcggtgctgcgctccttccgcctgctgcgggtcttcaagctggccaaatcatggcccaccctgaacacactcatcaagatcatcgggaactcagtgggggcactggggaacctgacactggtgctagccatcatcgtgttcatctttgctgtggtgggcatgcagctctttggcaagaactactcggagctgagggacagcgactcaggcctgctgcctcgctggcacatgatggacttctttcatgccttcctcatcatcttccgcatcctctgtggagagtggatcgagaccatgtgggactgcatggaggtgtcggggcagtcattatgcctgctggtcttcttgcttgttatggtcattggcaaccttgtggtcctgaatctcttcctggccttgctgctcagctccttcagtgcagacaacctcacagcccctgatgaggacagagagatgaacaacctccagctggccctggcccgcatccagaggggcctgcgctttgtcaagcggaccacctgggatttctgctgtggtctcctgcggcagcggcctcagaagcccgcagcccttgccgcccagggccagctgcccagctgcattgccaccccctactccccgccacccccagagacggagaaggtgcctcccacccgcaaggaaacacggtttgaggaaggcgagcaaccaggccagggcacccccggggatccagagcccgtgtgtgtgcccatcgctgtggccgagtcagacacagatgaccaagaagaagatgaggagaacagcctgggcacggaggaggagtccagcaagcagcaggaatcccagcctgtgtccggtggcccagaggcccctccggattccaggacctggagccaggtgtcagcgactgcctcctctgaggccgaggccagtgcatctcaggccgactggcggcagcagtggaaagcggaaccccaggccccagggtgcggtgagaccccagaggacagttgctccgagggcagcacagcagacatgaccaacaccgctgagctcctggagcagatccctgacctcggccaggatgtcaaggacccagaggactgcttcactgaaggctgtgtccggcgctgtccctgctgtgcggtggacaccacacaggccccagggaaggtctggtggcggttgcgcaagacctgctaccacatcgtggagcacagctggttcgagacattcatcatcttcatgatcctactcagcagtggagcgctggccttcgaggacatctacctagaggagcggaagaccatcaaggttctgcttgagtatgccgacaagatgttcacatatgtcttcgtgctggagatgctgctcaagtgggtggcctacggcttcaagaagtacttcaccaatgcctggtgctggctcgacttcctcatcgtagacgtctctctggtcagcctggtggccaacaccctgggctttgccgagatgggccccatcaagtcactgcggacgctgcgtgcactccgtcctctgagagctctgtcacgatttgagggcatgagggtggtggtcaatgccctggtgggcgccatcccgtccatcatgaacgtcctcctcgtctgcctcatcttctggctcatcttcagcatcatgggcgtgaacctctttgcggggaagtttgggaggtgcatcaaccagacagagggagacttgcctttgaactacaccatcgtgaacaacaagagccagtgtgagtccttgaacttgaccggagaattgtactggaccaaggtgaaagtcaactttgacaacgtgggggccgggtacctggcccttctgcaggtggcaacatttaaaggctggatggacattatgtatgcagctgtggactccagggggtatgaagagcagcctcagtgggaatacaacctctacatgtacatctattttgtcattttcatcatctttgggtctttcttcaccctgaacctctttattggtgtcatcattgacaacttcaaccaacagaagaaaaagttagggggccaggacatcttcatgacagaggagcagaagaagtactacaatgccatgaagaagctgggctccaagaagccccagaagcccatcccacggcccctgaacaagtaccagggcttcatattcgacattgtgaccaagcaggcctttgacgtcaccatcatgtttctgatctgcttgaatatggtgaccatgatggtggagacagatgaccaaagtcctgagaaaatcaacatcttggccaagatcaacctgctctttgtggccatcttcacaggcgagtgtattgtcaagctggctgccctgcgccactactacttcaccaacagctggaatatcttcgacttcgtggttgtcatcctctccatcgtgggcactgtgctctcggacatcatccagaagtacttcttctccccgacgctcttccgagtcatccgcctggcccgaataggccgcatcctcagactgatccgaggggccaaggggatccgcacgctgctctttgccctcatgatgtccctgcctgccctcttcaacatcgggctgctgctcttcctcgtcatgttcatctactccatctttggcatggccaacttcgcttatgtcaagtgggaggctggcatcgacgacatgttcaacttccagaccttcgccaacagcatgctgtgcctcttccagatcaccacgtcggccggctgggatggcctcctcagccccatcctcaacactgggccgccctactgcgaccccactctgcccaacagcaatggctctcggggggactgcgggagcccagccgtgggcatcctcttcttcaccacctacatcatcatctccttcctcatcgtggtcaacatgtacattgccatcatcctggagaacttcagcgtggccacggaggagagcaccgagcccctgagtgaggacgacttcgatatgttctatgagatctgggagaaatttgacccagaggccactcagtttattgagtattcggtcctgtctgactttgccgatgccctgtctgagccactccgtatcgccaagcccaaccagataagcctcatcaacatggacctgcccatggtgagtggggaccgcatccattgcatggacattctctttgccttcaccaaaagggtcctgggggagtctggggagatggacgccctgaagatccagatggaggagaagttcatggcagccaacccatccaagatctcctacgagcccatcaccaccacactccggcgcaagcacgaagaggtgtcggccatggttatccagagagccttccgcaggcacctgctgcaacgctctttgaagcatgcctccttcctcttccgtcagcaggcgggcagcggcctctccgaagaggatgcccctgagcgagagggcctcatcgcctacgtgatgagtgagaacttctcccgaccccttggcccaccctccagctcctccatctcctccacttccttcccaccctcctatgacagtgtcactagagccaccagcgataacctccaggtgcgggggtctgactacagccacagtgaagatctcgccgacttccccccttctccggacagggaccgtgagtccatcgtgtga

SEQ ID NO: 11 - DNA, 5049 bp, Homo sapiens
atgttggcttcaccagaacctaagggccttgttcccttcactaaagagtcttttgaacttataaaacagcatattgctaaaacacataatgaagaccatgaagaagaagacttaaagccaactcctgatttggaagttggcaaaaagcttccatttatttatggaaacctttctcaaggaatggtgtcagagcccttggaagatgtggacccatattactacaagaaaaaaaatactttcatagtattaaataaaaatagaacaatcttcagattcaatgcggcttccatcttgtgtacattgtctcctttcaattgtattagaagaacaactatcaaggttttggtacatccctttttccaactgtttattctaattagtgtcctgattgattgcgtattcatgtccctgactaatttgccaaaatggagaccagtattagagaatactttgcttggaatttacacatttgaaatacttgtaaaactctttgcaagaggtgtctgggcaggatcattttccttcctcggtgatccatggaactggctcgatttcagcgtaactgtgtttgaggttattataagatactcacctctggacttcattccaacgcttcaaactgcaagaactttgagaattttaaaaattattcctttaaatcaaggtctgaaatcccttgtaggggtcctgatccactgcttgaagcagcttattggtgtcattatcctaactctgttttttctgagcatattttctctaattgggatggggctcttcatgggcaacttgaaacataaatgttttcgatggccccaagagaatgaaaatgaaaccctgcacaacagaactggaaacccatattatattcgagaaacagaaaacttttattatttggaaggagaaagatatgctctcctttgtggcaacaggacagatgctggtcagtgtcctgaaggatatgtgtgtgtaaaagctggcataaatcctgatcaaggcttcacaaattttgacagttttggctgggccttatttgccctatttcggttaatggctcaggattaccctgaagtactttatcaccagatactttatgcttctgggaaggtctacatgatattttttgtggtggtaagttttttgttttccttttatatggcaagtttgttcttaggcatacttgccatggcctatgaagaagaaaagcagagagttggtgaaatatctaagaagattgaaccaaaatttcaacagactggaaaagaacttcaagaaggaaatgaaacagatgaggccaagaccatacaaatagaaatgaagaaaaggtcaccaatttccacagacacatcattggatgtgttggaagatgctactctcagacataaggaagaacttgaaaaatccaagaagatatgcccattatactggtataagtttgctaaaactttcttgatctggaattgttctccctgttggttaaaattgaaagagtttgtccataggattataatggcaccatttactgatcttttccttatcatatgcataattttaaacgtatgttttctgaccttggagcattatccaatgagtaaacaaactaacactcttctcaacattggaaacctggttttcattggaattttcacagcagaaatgatttttaaaataattgcaatgcatccatatgggtatttccaagtaggttggaacatttttgatagcatgatagtgttccatggtttaatagaactttgtctagcaaatgttgcaggaatggctcttcttcgattattcaggatgttaagaattttcaagttgggaaagtattggccaacattccagattttgatgtggtctcttagtaactcatgggtggccctgaaagacttggtcctgttgttgttcacattcatcttcttttctgctgcattcggcatgaagctgtttggtaagaattatgaagaatttgtctgccacatagacaaagactgtcaactcccacgctggcacatgcatgactttttccactccttcctgaatgtgttccgaattctctgtggagagtgggtagagaccttgtgggactgtatggaggttgcaggccaatcctggtgtattcctttttacctgatggtcattttaattggaaatttactggtactttacctgtttctggcattggtgagctcatttagttcatgcaaggatgtaacagctgaagagaataatgaagcaaaaaatctccagcttgcagtggcaagaattaaaaaaggaataaactatgtgcttcttaaaatactatgcaaaacacaaaatgtcccaaaggacacaatggaccatgtaaatgaggtatatgttaaagaagatatttctgaccataccctttctgaattgagcaacacccaagattttctcaaagataaggaaaaaagcagtggcacagagaaaaacgctactgaaaatgagagccaatcacttatccccagtcctagtgtctcagaaactgtaccaattgcttcaggagaatctgatatagaaaatctggataataaggagattcagagtaagtctggtgatggaggcagcaaagagaaaataaagcaatctagctcatctgaatgcagtactgttgatattgctatctctgaagaagaagaaatgttctatggaggtgaaagatcaaagcatctgaaaaatggttgcagacgcggatcttcacttggtcaaatcagtggagcatccaagaaaggaaaaatctggcagaacatcaggaaaacctgctgcaagattgtagagaacaattggtttaagtgttttattgggcttgttactctgctcagcactggcactctggcttttgaagatatatatatggatcagagaaagacaattaaaattttattagaatatgctgacatgatctttacttatatcttcattctggaaatgcttctaaaatggatggcatatggttttaaggcctatttctctaatggctggtacaggctggacttcgtggttgttattgtgttttgtcttagcttaataggcaaaactcgggaagaactaaaacctcttatttccatgaaattccttcggcccctcagagttctatctcaatttgaaagaatgaaggtggttgtgagagctttgatcaaaacaaccttacccactttgaatgtgtttcttgtctgcctgatgatctggctgatttttagtatcatgggagtagacttatttgctggcagattctatgaatgcattgacccaacaagtggagaaaggtttccttcatctgaagtcatgaataagagtcggtgtgaaagccttctgtttaacgaatccatgctatgggaaaatgcaaaaatgaactttgataatgttggaaatggtttcctttctctgcttcaagtagcaacatttaatggatggatcactattatgaattcagcaattgattctgttgctgttaatatacagcctcattttgaagtcaacatctacatgtattgttactttatcaactttattatatttggagtatttctccctctgagtatgctgattactgttattattgataatttcaacaagcataaaataaagctgggaggctcaaatatctttataacggttaaacagagaaaacagtaccgcaggctgaagaagctaatgtatgaggattctcaaagaccagtacctcgcccattaaacaagctccaaggattcatctttgatgtggtaacaagccaagcttttaatgtcattgttatggttcttatatgtttccaagcaatagccatgatgatagacactgatgttcagagtctacaaatgtccattgctctctactggattaactcaatttttgttatgctatatactatggaatgtatactgaagctcatcgctttccgttgtttttatttcaccattgcgtggaacatttttgattttatggtggttattttctccatcacaggactatgtctgcctatgacagtaggatcctaccttgtgcctccttcacttgtgcaactgatacttctctcacggatcattcacatgctgcgtcttggaaaaggaccaaaggtgtttcataatctgatgcttcctttgatgctgtccctcccagcattattgaacatcattcttctcatcttcctggtcatgttcatctatgccgtatttggaatgtataattttgcctatgttaaaaaagaagctggaattaatgatgtgtctaattttgaaacctttggcaacagtatgctctgtctttttcaagttgcaatatttgctggttgggatgggatgcttgatgcaattttcaacagtaaatggtctgactgtgatcctgataaaattaaccctgggactcaagttagaggagattgtgggaacccctctgttgggattttttattttgtcagttatatcctcatatcatggctgatcattgtaaatatgtacattgttgttgtcatggagtttttaaatattgcttctaagaagaaaaacaagaccttgagtgaagatgattttaggaaattctttcaggtatggaaaaggtttgatcctgataggacccagtacatagactctagcaagctttcagattttgcagctgctcttgatcctcctcttttcatggcaaaaccaaacaagggccagctcattgctttggacctccccatggctgttggggacagaattcattgcctcgatatcttacttgcttttacaaagagagttatgggtcaagatgtgaggatggagaaagttgtttcagaaatagaatcagggtttttgttagccaacccttttaagatcacatgtgagccaattacgactactttgaaacgaaaacaagaggcagtttcagcaaccatcattcaacgtgcttataaaaattaccgcttgaggcgaaatgacaaaaatacatcagatattcatatgatagatggtgacagagatgttcatgctactaaagaaggtgcctattttgacaaagctaaggaaaagtcacctattcaaagccagatctaa

SEQ ID NO: 12 - DNA, 5943 bp, Homo sapiens
atggcagcgcggctgcttgcaccaccaggccctgatagtttcaagcctttcacccctgagtcactggcaaacattgagaggcgcattgctgagagcaagctcaagaaaccaccaaaggccgatggcagtcatcgggaggacgatgaggacagcaagcccaagccaaacagcgacctggaagcagggaagagtttgcctttcatctacggggacatcccccaaggcctggttgcagttcccctggaggactttgacccatactatttgacgcagaaaacctttgtagtattaaacagagggaaaactctcttcagatttagtgccacgcctgccttgtacattttaagtccttttaacctgataagaagaatagctattaaaattttgatacattcagtatttagcatgatcattatgtgcactattttgaccaactgtgtattcatgacttttagtaaccctcctgactggtcgaagaatgtggagtacacgttcacagggatttatacatttgaatcactagtgaaaatcattgcaagaggtttctgcatagatggctttacctttttacgggacccatggaactggttagatttcagtgtcatcatgatggcgtatataacagagtttgtaaacctaggcaatgtttcagctctacgcactttcagggtactgagggctttgaaaactatttcggtaatcccaggcctgaagacaattgtgggtgccctgattcagtctgtgaagaaactgtcagatgtgatgatcctgacagtgttctgcctgagtgtttttgccttgatcggactgcagctgttcatggggaaccttcgaaacaagtgtgttgtgtggcccataaacttcaacgagagctatcttgaaaatggcaccaaaggctttgattgggaagagtatatcaacaataaaacaaatttctacacagttcctggcatgctggaacctttactctgtgggaacagttctgatgctgggcaatgcccagagggataccagtgtatgaaagcaggaaggaaccccaactatggttacacaagttttgacacttttagctgggccttcttggcattatttcgccttatgacccaggactattgggaaaacttgtatcaattgactttacgagcagccgggaaaacatacatgatcttcttcgtcttggtcatctttgtgggttctttctatctggtgaacttgatcttggctgtggtggccatggcttatgaagaacagaatcaggcaacactggaggaggcagaacaaaaagaggctgaatttaaagcaatgttggagcaacttaagaagcaacaggaagaggcacaggctgctgcgatggccacttcagcaggaactgtctcagaagatgccatagaggaagaaggtgaagaaggagggggctcccctcggagctcttctgaaatctctaaactcagctcaaagagtgcaaaggaaagacgtaacaggagaaagaagaggaagcaaaaggaactctctgaaggagaggagaaaggggatcccgagaaggtgtttaagtcagagtcagaagatggcatgagaaggaaggcctttcggctgccagacaacagaatagggaggaaattttccatcatgaatcagtcactgctcagcatcccaggctcgcccttcctctcccgccacaacagcaagagcagcatcttcagtttcaggggacctgggcggttccgagacccgggctccgagaatgagttcgcggatgacgagcacagcacggtggaggagagcgagggccgccgggactccctcttcatccccatccgggcccgcgagcgccggagcagctacagcggctacagcggctacagccagggcagccgctcctcgcgcatcttccccagcctgcggcgcagcgtgaagcgcaacagcacggtggactgcaacggcgtggtgtccctcatcggcggccccggctcccacatcggcgggcgtctcctgccagaggctacaactgaggtggaaattaagaagaaaggccctggatctcttttagtttccatggaccaattagcctcctacgggcggaaggacagaatcaacagtataatgagtgttgttacaaatacactagtagaagaactggaagagtctcagagaaagtgcccgccatgctggtataaatttgccaacactttcctcatctgggagtgccacccctactggataaaactgaaagagattgtgaacttgatagttatggacccttttgtggatttagccatcaccatctgcatcgtcctgaatacactgtttatggcaatggagcaccatcctatgacaccacaatttgaacatgtcttggctgtaggaaatctggttttcactggaattttcacagcggaaatgttcctgaagctcatagccatggatccctactattatttccaagaaggttggaacatttttgacggatttattgtctccctcagtttaatggaactgagtctagcagacgtggaggggctttcagtgctgcgatctttccgattgctccgagtcttcaaattggccaaatcctggcccaccctgaacatgctaatcaagattattggaaattcagtgggtgccctgggcaacctgacactggtgctggccattattgtcttcatctttgccgtggtggggatgcaactctttggaaaaagctacaaagagtgtgtctgcaagatcaaccaggactgtgaactccctcgctggcatatgcatgactttttccattccttcctcattgtctttcgagtgttgtgcggggagtggattgagaccatgtgggactgcatggaagtggcaggccaggccatgtgcctcattgtctttatgatggtcatggtgattggcaacttggtggtgctgaacctgtttctggccttgctcctgagctccttcagtgcagacaacctggctgccacagatgacgatggggaaatgaacaacctccagatctcagtgatccgtatcaagaagggtgtggcctggaccaaactaaaggtgcacgccttcatgcaggcccactttaagcagcgtgaggctgatgaggtgaagcctctggatgagttgtatgaaaagaaggccaactgtatcgccaatcacaccggtgcagacatccaccggaatggtgacttccagaagaatggcaatggcacaaccagcggcattggcagcagcgtggagaagtacatcattgatgaggaccacatgtccttcatcaacaaccccaacttgactgtacgggtacccattgctgtgggcgagtctgactttgagaacctcaacacagaggatgttagcagcgagtcggatcctgaaggcagcaaagataaactagatgacaccagctcctctgaaggaagcaccattgatatcaaaccagaagtagaagaggtccctgtggaacagcctgaggaatacttggatccagatgcctgcttcacagaaggttgtgtccagcggttcaagtgctgccaggtcaacatcgaggaagggctaggcaagtcttggtggatcctgcggaaaacctgcttcctcatcgtggagcacaactggtttgagaccttcatcatcttcatgattctgctgagcagtggcgccctggccttcgaggacatctacattgagcagagaaagaccatccgcaccatcctggaatatgctgacaaagtcttcacctatatcttcatcctggagatgttgctcaagtggacagcctatggcttcgtcaagttcttcaccaatgcctggtgttggctggacttcctcattgtggctgtctctttagtcagccttatagctaatgccctgggctactcggaactaggtgccataaagtcccttaggaccctaagagctttgagacccttaagagccttatcacgatttgaagggatgagggtggtggtgaatgccttggtgggcgccatcccctccatcatgaatgtgctgctggtgtgtctcatcttctggctgattttcagcatcatgggagttaacttgtttgcgggaaagtaccactactgctttaatgagacttctgaaatccgatttgaaattgaagatgtcaacaataaaactgaatgtgaaaagcttatggaggggaacaatacagagatcagatggaagaacgtgaagatcaactttgacaatgttggggcaggatacctggcccttcttcaagtagcaaccttcaaaggctggatggacatcatgtatgcagctgtagattcccggaagcctgatgagcagcctaagtatgaggacaatatctacatgtacatctattttgtcatcttcatcatcttcggctccttcttcaccctgaacctgttcattggtgtcatcattgataacttcaatcaacaaaagaaaaagttcggaggtcaggacatcttcatgaccgaagaacagaagaagtactacaatgccatgaaaaagctgggctcaaagaagccacagaaacctattccccgccccttgaacaaaatccaaggaatcgtctttgattttgtcactcagcaagcctttgacattgttatcatgatgctcatctgccttaacatggtgacaatgatggtggagacagacactcaaagcaagcagatggagaacatcctctactggattaacctggtgtttgttatcttcttcacctgtgagtgtgtgctcaaaatgtttgcgttgaggcactactacttcaccattggctggaacatcttcgacttcgtggtagtcatcctctccattgtgggaatgttcctggcagatataattgagaaatactttgtttccccaaccctattccgagtcatccgattggcccgtattgggcgcatcttgcgtctgatcaaaggcgccaaagggattcgtaccctgctctttgccttaatgatgtccttgcctgccctgttcaacatcggccttctgctcttcctggtcatgttcatcttctccatttttgggatgtccaattttgcatatgtgaagcacgaggctggtatcgatgacatgttcaactttgagacatttggcaacagcatgatctgcctgtttcaaatcacaacctcagctggttgggatggcctgctgctgcccatcctaaaccgcccccctgactgcagcctagataaggaacacccagggagtggctttaagggagattgtgggaacccctcagtgggcatcttcttctttgtaagctacatcatcatctctttcctaattgtcgtgaacatgtacattgccatcatcctggagaacttcagtgtagccacagaggaaagtgcagaccctctgagtgaggatgactttgagaccttctatgagatctgggagaagttcgaccccgatgccacccagttcattgagtactgtaagctggcagactttgcagatgccttggagcatcctctccgagtgcccaagcccaataccattgagctcatcgctatggatctgccaatggtgagcggggatcgcatccactgcttggacatcctttttgccttcaccaagcgggtcctgggagatagcggggagttggacatcctgcggcagcagatggaagagcggttcgtggcatccaatccttccaaagtgtcttacgagccaatcacaaccacactgcgtcgcaagcaggaggaggtatctgcagtggtcctgcagcgtgcctaccggggacatttggcaaggcggggcttcatctgcaaaaagacaacttctaataagctggagaatggaggcacacaccgggagaaaaaagagagcaccccatctacagcctccctcccgtcctatgacagtgtaactaaacctgaaaaggagaaacagcagcgggcagaggaaggaagaagggaaagagccaaaagacaaaaagaggtcagagaatccaagtgttag

SEQ ID NO: 13 - DNA, 5934 bp, Homo sapiens
atggcaatgttgcctcccccaggacctcagagctttgtccatttcacaaaacagtctcttgccctcattgaacaacgcattgctgaaagaaaatcaaaggaacccaaagaagaaaagaaagatgatgatgaagaagccccaaagccaagcagtgacttggaagctggcaaacaactgcccttcatctatggggacattcctcccggcatggtgtcagagcccctggaggacttggacccctactatgcagacaaaaagactttcatagtattgaacaaagggaaaacaatcttccgtttcaatgccacacctgctttatatatgctttctcctttcagtcctctaagaagaatatctattaagattttagtacactccttattcagcatgctcatcatgtgcactattctgacaaactgcatatttatgaccatgaataacccgccggactggaccaaaaatgtcgagtacacttttactggaatatatacttttgaatcacttgtaaaaatccttgcaagaggcttctgtgtaggagaattcacttttcttcgtgacccgtggaactggctggattttgtcgtcattgtttttgcgtatttaacagaatttgtaaacctaggcaatgtttcagctcttcgaactttcagagtattgagagctttgaaaactatttctgtaatcccaggcctgaagacaattgtaggggctttgatccagtcagtgaagaagctttctgatgtcatgatcctgactgtgttctgtctgagtgtgtttgcactaattggactacagctgttcatgggaaacctgaagcataaatgttttcgaaattcacttgaaaataatgaaacattagaaagcataatgaataccctagagagtgaagaagactttagaaaatatttttattacttggaaggatccaaagatgctctcctttgtggtttcagcacagattcaggtcagtgtccagaggggtacacctgtgtgaaaattggcagaaaccctgattatggctacacgagctttgacactttcagctgggccttcttagccttgtttaggctaatgacccaagattactgggaaaacctttaccaacagacgctgcgtgctgctggcaaaacctacatgatcttctttgtcgtagtgattttcctgggctccttttatctaataaacttgatcctggctgtggttgccatggcatatgaagaacagaaccaggcaaacattgaagaagctaaacagaaagaattagaatttcaacagatgttagaccgtcttaaaaaagagcaagaagaagctgaggcaattgcagcggcagcggctgaatatacaagtattaggagaagcagaattatgggcctctcagagagttcttctgaaacatccaaactgagctctaaaagtgctaaagaaagaagaaacagaagaaagaaaaagaatcaaaagaagctctccagtggagaggaaaagggagatgctgagaaattgtcgaaatcagaatcagaggacagcatcagaagaaaaagtttccaccttggtgtcgaagggcataggcgagcacatgaaaagaggttgtctacccccaatcagtcaccactcagcattcgtggctccttgttttctgcaaggcgaagcagcagaacaagtctttttagtttcaaaggcagaggaagagatataggatctgagactgaatttgccgatgatgagcacagcatttttggagacaatgagagcagaaggggctcactgtttgtgccccacagaccccaggagcgacgcagcagtaacatcagccaagccagtaggtccccaccaatgctgccggtgaacgggaaaatgcacagtgctgtggactgcaacggtgtggtctccctggttgatggacgctcagccctcatgctccccaatggacagcttctgccagagggcacgaccaatcaaatacacaagaaaaggcgttgtagttcctatctcctttcagaggatatgctgaatgatcccaacctcagacagagagcaatgagtagagcaagcatattaacaaacactgtggaagaacttgaagagtccagacaaaaatgtccaccttggtggtacagatttgcacacaaattcttgatctggaattgctctccatattggataaaattcaaaaagtgtatctattttattgtaatggatccttttgtagatcttgcaattaccatttgcatagttttaaacacattatttatggctatggaacaccacccaatgactgaggaattcaaaaatgtacttgctataggaaatttggtctttactggaatctttgcagctgaaatggtattaaaactgattgccatggatccatatgagtatttccaagtaggctggaatatttttgacagccttattgtgactttaagtttagtggagctctttctagcagatgtggaaggattgtcagttctgcgatcattcagactgctccgagtcttcaagttggcaaaatcctggccaacattgaacatgctgattaagatcattggtaactcagtaggggctctaggtaacctcaccttagtgttggccatcatcgtcttcatttttgctgtggtcggcatgcagctctttggtaagagctacaaagaatgtgtctgcaagatcaatgatgactgtacgctcccacggtggcacatgaacgacttcttccactccttcctgattgtgttccgcgtgctgtgtggagagtggatagagaccatgtgggactgtatggaggtcgctggtcaagctatgtgccttattgtttacatgatggtcatggtcattggaaacctggtggtcctaaacctatttctggccttattattgagctcatttagttcagacaatcttacagcaattgaagaagaccctgatgcaaacaacctccagattgcagtgactagaattaaaaagggaataaattatgtgaaacaaaccttacgtgaatttattctaaaagcattttccaaaaagccaaagatttccagggagataagacaagcagaagatctgaatactaagaaggaaaactatatttctaaccatacacttgctgaaatgagcaaaggtcacaatttcctcaaggaaaaagataaaatcagtggttttggaagcagcgtggacaaacacttgatggaagacagtgatggtcaatcatttattcacaatcccagcctcacagtgacagtgccaattgcacctggggaatccgatttggaaaatatgaatgctgaggaacttagcagtgattcggatagtgaatacagcaaagtgagattaaaccggtcaagctcctcagagtgcagcacagttgataaccctttgcctggagaaggagaagaagcagaggctgaacctatgaattccgatgagccagaggcctgtttcacagatggttgtgtacggaggttctcatgctgccaagttaacatagagtcagggaaaggaaaaatctggtggaacatcaggaaaacctgctacaagattgttgaacacagttggtttgaaagcttcattgtcctcatgatcctgctcagcagtggtgccctggcttttgaagatatttatattgaaaggaaaaagaccattaagattatcctggagtatgcagacaagatcttcacttacatcttcattctggaaatgcttctaaaatggatagcatatggttataaaacatatttcaccaatgcctggtgttggctggatttcctaattgttgatgtttctttggttactttagtggcaaacactcttggctactcagatcttggccccattaaatcccttcggacactgagagctttaagacctctaagagccttatctagatttgaaggaatgagggtcgttgtgaatgcactcataggagcaattccttccatcatgaatgtgctacttgtgtgtcttatattctggctgatattcagcatcatgggagtaaatttgtttgctggcaagttctatgagtgtattaacaccacagatgggtcacggtttcctgcaagtcaagttccaaatcgttccgaatgttttgcccttatgaatgttagtcaaaatgtgcgatggaaaaacctgaaagtgaactttgataatgtcggacttggttacctatctctgcttcaagttgcaacttttaagggatggacgattattatgtatgcagcagtggattctgttaatgtagacaagcagcccaaatatgaatatagcctctacatgtatatttattttgtcgtctttatcatctttgggtcattcttcactttgaacttgttcattggtgtcatcatagataatttcaaccaacagaaaaagaagcttggaggtcaagacatctttatgacagaagaacagaagaaatactataatgcaatgaaaaagctggggtccaagaagccacaaaagccaattcctcgaccagggaacaaaatccaaggatgtatatttgacctagtgacaaatcaagcctttgatattagtatcatggttcttatctgtctcaacatggtaaccatgatggtagaaaaggagggtcaaagtcaacatatgactgaagttttatattggataaatgtggtttttataatccttttcactggagaatgtgtgctaaaactgatctccctcagacactactacttcactgtaggatggaatatttttgattttgtggttgtgattatctccattgtaggtatgtttctagctgatttgattgaaacgtattttgtgtcccctaccctgttccgagtgatccgtcttgccaggattggccgaatcctacgtctagtcaaaggagcaaaggggatccgcacgctgctctttgctttgatgatgtcccttcctgcgttgtttaacatcggcctcctgctcttcctggtcatgttcatctacgccatctttggaatgtccaactttgcctatgttaaaaaggaagatggaattaatgacatgttcaattttgagacctttggcaacagtatgatttgcctgttccaaattacaacctctgctggctgggatggattgctagcacctattcttaacagtaagccacccgactgtgacccaaaaaaagttcatcctggaagttcagttgaaggagactgtggtaacccatctgttggaatattctactttgttagttatatcatcatatccttcctggttgtggtgaacatgtacattgcagtcatactggagaattttagtgttgccactgaagaaagtactgaacctctgagtgaggatgactttgagatgttctatgaggtttgggagaagtttgatcccgatgcgacccagtttatagagttctctaaactctctgattttgcagctgccctggatcctcctcttctcatagcaaaacccaacaaagtccagctcattgccatggatctgcccatggttagtggtgaccggatccattgtcttgacatcttatttgcttttacaaagcgtgttttgggtgagagtggggagatggattctcttcgttcacagatggaagaaaggttcatgtctgcaaatccttccaaagtgtcctatgaacccatcacaaccacactaaaacggaaacaagaggatgtgtctgctactgtcattcagcgtgcttatagacgttaccgcttaaggcaaaatgtcaaaaatatatcaagtatatacataaaagatggagacagagatgatgatttactcaataaaaaagatatggcttttgataatgttaatgagaactcaagtccagaaaaaacagatgccacttcatccaccacctctccaccttcatatgatagtgtaacaaagccagacaaagagaaatatgaacaagacagaacagaaaaggaagacaaagggaaagacagcaaggaaagcaaaaaatag

SEQ ID NO: 14 - DNA, 5871 bp, Homo sapiens
atggaattccccattggatccctcgaaactaacaacttccgtcgctttactccggagtcactggtggagatagagaagcaaattgctgccaagcagggaacaaagaaagccagagagaagcatagggagcagaaggaccaagaagagaagcctcggccccagctggacttgaaagcctgcaaccagctgcccaagttctatggtgagctcccagcagaactgatcggggagcccctggaggatctagatccgttctacagcacacaccggacatttatggtgctgaacaaagggaggaccatttcccggtttagtgccactcgggccctgtggctattcagtcctttcaacctgatcagaagaacggccatcaaagtgtctgtccactcgtggttcagtttatttattacggtcactattttggttaattgtgtgtgcatgacccgaactgaccttccagagaaaattgaatatgtcttcactgtcatttacacctttgaagccttgataaagatactggcaagaggattttgtctaaatgagttcacgtacctgagagatccttggaactggctggattttagcgtcattaccctggcatatgttggcacagcaatagatctccgtgggatctcaggcctgcggacattcagagttcttagagcattaaaaacagtttctgtgatcccaggcctgaaggtcattgtgggggccctgattcactcagtgaagaaactggctgatgtgaccatcctcaccatcttctgcctaagtgtttttgccttggtggggctgcaactcttcaagggcaacctcaaaaataaatgtgtcaagaatgacatggctgtcaatgagacaaccaactactcatctcacagaaaaccagatatctacataaataagcgaggcacttctgaccccttactgtgtggcaatggatctgactcaggccactgccctgatggttatatctgccttaaaacttctgacaacccggattttaactacaccagctttgattcctttgcttgggctttcctctcactgttccgcctcatgacacaggattcctgggaacgcctctaccagcagaccctgaggacttctgggaaaatctatatgatcttttttgtgctcgtaatcttcctgggatctttctacctggtcaacttgatcttggctgtagtcaccatggcgtatgaggagcagaaccaggcaaccactgatgaaattgaagcaaaggagaagaagttccaggaggccctcgagatgctccggaaggagcaggaggtgctagcagcactagggattgacacaacctctctccactcccacaatggatcacctttaacctccaaaaatgccagtgagagaaggcatagaataaagccaagagtgtcagagggctccacagaagacaacaaatcaccccgctctgatccttacaaccagcgcaggatgtcttttctaggcctcgcctctggaaaacgccgggctagtcatggcagtgtgttccatttccggtcccctggccgagatatctcactccctgagggagtcacagatgatggagtctttcctggagaccacgaaagccatcggggctctctgctgctgggtgggggtgctggccagcaaggccccctccctagaagccctcttcctcaacccagcaaccctgactccaggcatggagaagatgaacaccaaccgccgcccactagtgagcttgcccctggagctgtcgatgtctcggcattcgatgcaggacaaaagaagactttcttgtcagcagaatacttagatgaacctttccgggcccaaagggcaatgagtgttgtcagtatcataacctccgtccttgaggaactcgaggagtctgaacagaagtgcccaccctgcttgaccagcttgtctcagaagtatctgatctgggattgctgccccatgtgggtgaagctcaagacaattctctttgggcttgtgacggatccctttgcagagctcaccatcaccttgtgcatcgtggtgaacaccatcttcatggccatggagcaccatggcatgagccctaccttcgaagccatgctccagataggcaacatcgtctttaccatattttttactgctgaaatggtcttcaaaatcattgccttcgacccatactattatttccagaagaagtggaatatctttgactgcatcatcgtcactgtgagtctgctagagctgggcgtggccaagaagggaagcctgtctgtgctgcggagcttccgcttgctgcgcgtattcaagctggccaaatcctggcccaccttaaacacactcatcaagatcatcggaaactcagtgggggcactggggaacctcaccatcatcctggccatcattgtctttgtctttgctctggttggcaagcagctcctaggggaaaactaccgtaacaaccgaaaaaatatctccgcgccccatgaagactggccccgctggcacatgcacgacttcttccactctttcctcattgtcttccgtatcctctgtggagagtggattgagaacatgtgggcctgcatggaagttggccaaaaatccatatgcctcatccttttcttgacggtgatggtgctagggaacctggtggtgcttaacctgttcatcgccctgctattgaactctttcagtgctgacaacctcacagccccggaggacgatggggaggtgaacaacctgcaggtggccctggcacggatccaggtctttggccatcgtaccaaacaggctctttgcagcttcttcagcaggtcctgcccattcccccagcccaaggcagagcctgagctggtggtgaaactcccactctccagctccaaggctgagaaccacattgctgccaacactgccagggggagctctggagggctccaagctcccagaggccccagggatgagcacagtgacttcatcgctaatccgactgtgtgggtctctgtgcccattgctgagggtgaatctgatcttgatgacttggaggatgatggtggggaagatgctcagagcttccagcaggaagtgatccccaaaggacagcaggagcagctgcagcaagtcgagaggtgtggggaccacctgacacccaggagcccaggcactggaacatcttctgaggacctggctccatccctgggtgagacgtggaaagatgagtctgttcctcaggtccctgctgagggagtggacgacacaagctcctctgagggcagcacggtggactgcctagatcctgaggaaatcctgaggaagatccctgagctggcagatgacctggaagaaccagatgactgcttcacagaaggatgcattcgccactgtccctgctgcaaactggataccaccaagagtccatgggatgtgggctggcaggtgcgcaagacttgctaccgtatcgtggagcacagctggtttgagagcttcatcatcttcatgatcctgctcagcagtggatctctggcctttgaagactattacctggaccagaagcccacggtgaaagctttgctggagtacactgacagggtcttcacctttatctttgtgttcgagatgctgcttaagtgggtggcctatggcttcaaaaagtacttcaccaatgcctggtgctggctggacttcctcattgtgaatatctcactgataagtctcacagcgaagattctggaatattctgaagtggctcccatcaaagcccttcgaacccttcgcgctctgcggccactgcgggctctttctcgatttgaaggcatgcgggtggtggtggatgccctggtgggcgccatcccatccatcatgaatgtcctcctcgtctgcctcatcttctggctcatcttcagcatcatgggtgtgaacctcttcgcagggaagttttggaggtgcatcaactataccgatggagagttttcccttgtacctttgtcgattgtgaataacaagtctgactgcaagattcaaaactccactggcagcttcttctgggtcaatgtgaaagtcaactttgataatgttgcaatgggttaccttgcacttctgcaggtggcaacctttaaaggctggatggacattatgtatgcagctgttgattcccgggaggtcaacatgcaacccaagtgggaggacaacgtgtacatgtatttgtactttgtcatcttcatcatttttggaggcttcttcacactgaatctctttgttggggtcataattgacaacttcaatcaacagaaaaaaaagttagggggccaggacatcttcatgacagaggagcagaagaaatactacaatgccatgaagaagttgggctccaagaagccccagaagcccatcccacggcccctgaacaagttccagggttttgtctttgacatcgtgaccagacaagcttttgacatcaccatcatggtcctcatctgcctcaacatgatcaccatgatggtggagactgatgaccaaagtgaagaaaagacgaaaattctgggcaaaatcaaccagttctttgtggccgtcttcacaggcgaatgtgtcatgaagatgttcgctttgaggcagtactacttcacaaatggctggaatgtgtttgacttcattgtggtggttctctccattgcgagcctgattttttctgcaattcttaagtcacttcaaagttacttctccccaacgctcttcagagtcatccgcctggcccgaattggccgcatcctcagactgatccgagcggccaaggggatccgcacactgctctttgccctcatgatgtccctgcctgccctcttcaacatcgggctgttgctattccttgtcatgttcatctactctatcttcggtatgtccagctttccccatgtgaggtgggaggctggcatcgacgacatgttcaacttccagaccttcgccaacagcatgctgtgcctcttccagattaccacgtcggccggctgggatggcctcctcagccccatcctcaacacagggcccccctactgtgaccccaatctgcccaacagcaatggcaccagaggggactgtgggagcccagccgtaggcatcatcttcttcaccacctacatcatcatctccttcctcatcatggtcaacatgtacattgcagtgattctggagaacttcaatgtggccacggaggagagcactgagcccctgagtgaggacgactttgacatgttctatgagacctgggagaagtttgacccagaggccactcagtttattaccttttctgctctctcggactttgcagacactctctctggtcccctgagaatcccaaaacccaatcgaaatatactgatccagatggacctgcctttggtccctggagataagatccactgcttggacatcctttttgctttcaccaagaatgtcctaggagaatccggggagttggattctctgaaggcaaatatggaggagaagtttatggcaactaatctttcaaaatcatcctatgaaccaatagcaaccactctccgatggaagcaagaagacatttcagccactgtcattcaaaaggcctatcggagctatgtgctgcaccgctccatggcactctctaacaccccatgtgtgcccagagctgaggaggaggctgcatcactcccagatgaaggttttgttgcattcacagcaaatgaaaattgtgtactcccagacaaatctgaaactgcttctgccacatcattcccaccgtcctatgagagtgtcactagaggccttagtgatagagtcaacatgaggacatctagctcaatacaaaatgaagatgaagccaccagtatggagctgattgcccctgggccctag

SEQ ID NO: 15 - DNA, 5376 bp, Homo sapiens
atggatgacagatgctacccagtaatctttccagatgagcggaatttccgccccttcacttccgactctctggctgcaattgagaagcggattgccatccaaaaggagaaaaagaagtctaaagaccagacaggagaagtaccccagcctcggcctcagcttgacctaaaggcctccaggaagttgcccaagctctatggcgacattcctcgtgagctcataggaaagcctctggaagacttggacccattctaccgaaatcataagacatttatggtgttaaacagaaagaggacaatctaccgcttcagtgccaagcatgccttgttcatttttgggcctttcaattcaatcagaagtttagccattagagtctcagtccattcattgttcagcatgttcattatcggcaccgttatcatcaactgcgtgttcatggctacagggcctgctaaaaacagcaacagtaacaatactgacattgcagagtgtgtcttcactgggatttatatttttgaagctttgattaaaatattggcaagaggtttcattctggatgagttttctttccttcgagatccatggaactggctggactccattgtcattggaatagcgattgtgtcatatattccaggaatcaccatcaaactattgcccctgcgtaccttccgtgtgttcagagctttgaaagcaatttcagtagtttcacgtctgaaggtcatcgtgggggccttgctacgctctgtgaagaagctggtcaacgtgattatcctcaccttcttttgcctcagcatctttgccctggtaggtcagcagctcttcatgggaagtctgaacctgaaatgcatctcgagggactgtaaaaatatcagtaacccggaagcttatgaccattgctttgaaaagaaagaaaattcacctgaattcaaaatgtgtggcatctggatgggtaacagtgcctgttccatacaatatgaatgtaagcacaccaaaattaatcctgactataattatacgaattttgacaactttggctggtcttttcttgccatgttccggctgatgacccaagattcctgggagaagctttatcaacagaccctgcgtactactgggctctactcagtcttcttcttcattgtggtcattttcctgggctccttctacctgattaacttaaccctggctgttgttaccatggcatatgaggagcagaacaagaatgtagctgcagagatagaggccaaggaaaagatgtttcaggaagcccagcagctgttaaaggaggaaaaggaggctctggttgccatgggaattgacagaagttcacttacttcccttgaaacatcatattttaccccaaaaaagagaaagctctttggtaataagaaaaggaagtccttctttttgagagagtctgggaaagaccagcctcctgggtcagattctgatgaagattgccaaaaaaagccacagctcctagagcaaaccaaacgactgtcccagaatctatcactggaccactttgatgagcatggagatcctctccaaaggcagagagcactgagtgctgtcagcatcctcaccatcaccatgaaggaacaagaaaaatcacaagagccttgtctcccttgtggagaaaacctggcatccaagtacctcgtgtggaactgttgcccccagtggctgtgcgttaagaaggtcctgagaactgtgatgactgacccgtttactgagctggccatcaccatctgcatcatcatcaacactgtcttcttggccatggagcatcacaagatggaggccagttttgagaagatgttgaatatagggaatttggttttcactagcatttttatagcagaaatgtgcctaaaaatcattgcgctcgatccctaccactactttcgccgaggctggaacatttttgacagcattgttgctcttctgagttttgcagatgtaatgaactgtgtacttcaaaagagaagctggccattcttgcgttccttcagagtgctcagggtcttcaagttagccaaatcctggccaactttgaacacactaattaagataatcggcaactctgtcggagcccttggaagcctgactgtggtcctggtcattgtgatctttattttctcagtagttggcatgcagctttttggccgtagcttcaattcccaaaagagtccaaaactctgtaacccgacaggcccgacagtctcatgtttacggcactggcacatgggggatttctggcactccttcctagtggtattccgcatcctctgcggggaatggatcgaaaatatgtgggaatgtatgcaagaagcgaatgcatcatcatcattgtgtgttattgtcttcatattgatcacggtgataggaaaacttgtggtgctcaacctcttcattgccttactgctcaattcctttagcaatgaggaaagaaatggaaacttagaaggagaggccaggaaaactaaagtccagttagcactggatcgattccgccgggctttttgttttgtgagacacactcttgagcatttctgtcacaagtggtgcaggaagcaaaacttaccacagcaaaaagaggtggcaggaggctgtgctgcacaaagcaaagacatcattcccctggtcatggagatgaaaaggggctcagagacccaggaggagcttggtatactaacctctgtaccaaagaccctgggcgtcaggcatgattggacttggttggcaccacttgcggaggaggaagatgacgttgaattttctggtgaagataatgcacagcgcatcacacaacctgagcctgaacaacaggcctatgagctccatcaggagaacaagaagcccacgagccagagagttcaaagtgtggaaattgacatgttctctgaagatgagcctcatctgaccatacaggatccccgaaagaagtctgatgttaccagtatactatcagaatgtagcaccattgatcttcaggatggctttggatggttacctgagatggttcccaaaaagcaaccagagagatgtttgcccaaaggctttggttgctgctttccatgctgtagcgtggacaagagaaagcctccctgggtcatttggtggaacctgcggaaaacctgctaccaaatagtgaaacacagctggtttgagagctttattatctttgtgattctgctgagcagtggggcactgatatttgaagatgttcaccttgagaaccaacccaaaatccaagaattactaaattgtactgacattatttttacacatatttttatcctggagatggtactaaaatgggtagccttcggatttggaaagtatttcaccagtgcctggtgctgccttgatttcatcattgtgattgtctctgtgaccaccctcattaacttaatggaattgaagtccttccggactctacgagcactgaggcctcttcgtgcgctgtcccagtttgaaggaatgaaggtggtggtcaatgctctcataggtgccatacctgccattctgaatgttttgcttgtctgcctcattttctggctcgtattttgtattctgggagtatacttcttttctggaaaatttgggaaatgcattaatggaacagactcagttataaattataccatcattacaaataaaagtcaatgtgaaagtggcaatttctcttggatcaaccagaaagtcaactttgacaatgtgggaaatgcttacctcgctctgctgcaagtggcaacatttaagggctggatggatattatatatgcagctgttgattccacagagaaagaacaacagccagagtttgagagcaattcactcggttacatttacttcgtagtctttatcatctttggctcattcttcactctgaatctcttcattggcgttatcattgacaacttcaaccaacagcagaaaaagttaggtggccaagacatttttatgacagaagaacagaagaaatactataatgcaatgaaaaaattaggatccaaaaaacctcaaaaacccattccacggcctctgaacaaatgtcaaggtctcgtgttcgacatagtcacaagccagatctttgacatcatcatcataagtctcattatcctaaacatgattagcatgatggctgaatcatacaaccaacccaaagccatgaaatccatccttgaccatctcaactgggtctttgtggtcatctttacgttagaatgtctcatcaaaatctttgctttgaggcaatactacttcaccaatggctggaatttatttgactgtgtggtcgtgcttctttccattgttagtacaatgatttctaccttggaaaatcaggagcacattcctttccctccgacgctcttcagaattgtccgcttggctcggattggccgaatcctgaggcttgtccgggctgcacgaggaatcaggactctcctctttgctctgatgatgtcgcttccttctctgttcaacattggtcttctactctttctgattatgtttatctatgccattctgggtatgaactggttttccaaagtgaatccagagtctggaatcgatgacatattcaacttcaagacttttgccagcagcatgctctgtctcttccagataagcacatcagcaggttgggattccctgctcagccccatgctgcgatcaaaagaatcatgtaactcttcctcagaaaactgccacctccctggcatagccacatcctactttgtcagttacattatcatctcctttctcattgttgtcaacatgtacattgctgtgattttagagaacttcaatacagccactgaagaaagtgaggaccctttgggtgaagatgactttgacatattttatgaagtgtgggaaaagtttgacccagaagcaacacaatttatcaaatattctgccctttctgactttgctgatgccttgcctgagcctttgcgtgtcgcaaagccaaataaatatcaatttctagtaatggacttgcccatggtgagtgaagatcgcctccactgcatggatattcttttcgccttcaccgctagggtactcggtggctctgatggcctagatagtatgaaagcaatgatggaagagaagttcatggaagccaatcctctcaagaagttgtatgaacccatagtcaccaccaccaagagaaaggaagaggaaagaggtgctgctattattcaaaaggcctttcgaaagtacatgatgaaggtgaccaagggtgaccaaggtgaccaaaatgacttggaaaacgggcctcattcaccactccagactctttgcaatggagacttgtctagctttggggtggccaagggcaaggtccactgtgactga

SEQ ID NO: 16 - DNA, 657 bp, Homo sapiens
atggggaggctgctggccttagtggtcggcgcggcactggtgtcctcagcctgcgggggctgcgtggaggtggactcggagaccgaggccgtgtatgggatgaccttcaaaattctttgcatctcctgcaagcgccgcagcgagaccaacgctgagaccttcaccgagtggaccttccgccagaagggcactgaggagtttgtcaagatcctgcgctatgagaatgaggtgttgcagctggaggaggatgagcgcttcgagggccgcgtggtgtggaatggcagccggggcaccaaagacctgcaggatctgtctatcttcatcaccaatgtcacctacaaccactcgggcgactacgagtgccacgtctaccgcctgctcttcttcgaaaactacgagcacaacaccagcgtcgtcaagaagatccacattgaggtagtggacaaagccaacagagacatggcatccatcgtgtctgagatcatgatgtatgtgctcattgtggtgttgaccatatggctcgtggcagagatgatttactgctacaagaagatcgctgccgccacggagactgctgcacaggagaatgcctcggaatacctggccatcacctctgaaagcaaagagaactgcacgggcgtccaggtggccgaatag

SEQ ID NO: 17 - DNA, 648 bp, Homo sapiens
atgcacagagatgcctggctacctcgccctgccttcagcctcacggggctcagtctctttttctctttggtgccaccaggacggagcatggaggtcacagtacctgccaccctcaacgtcctcaatggctctgacgcccgcctgccctgcaccttcaactcctgctacacagtgaaccacaaacagttctccctgaactggacttaccaggagtgcaacaactgctctgaggagatgttcctccagttccgcatgaagatcattaacctgaagctggagcggtttcaagaccgcgtggagttctcagggaaccccagcaagtacgatgtgtcggtgatgctgagaaacgtgcagccggaggatgaggggatttacaactgctacatcatgaacccccctgaccgccaccgtggccatggcaagatccatctgcaggtcctcatggaagagccccctgagcgggactccacggtggccgtgattgtgggtgcctccgtcgggggcttcctggctgtggtcatcttggtgctgatggtggtcaagtgtgtgaggagaaaaaaagagcagaagctgagcacagatgacctgaagaccgaggaggagggcaagacggacggtgaaggcaacccggatgatggcgccaagtag

SEQ ID NO: 18 - DNA, 648 bp, Homo sapiens
atgcctgccttcaatagattgtttcccctggcttctctcgtgcttatctactgggtcagtgtctgcttccctgtgtgtgtggaagtgccctcggagacggaggccgtgcagggcaaccccatgaagctgcgctgcatctcctgcatgaagagagaggaggtggaggccaccacggtggtggaatggttctacaggcccgagggcggtaaagatttccttatttacgagtatcggaatggccaccaggaggtggagagcccctttcaggggcgcctgcagtggaatggcagcaaggacctgcaggacgtgtccatcactgtgctcaacgtcactctgaacgactctggcctctacacctgcaatgtgtcccgggagtttgagtttgaggcgcatcggccctttgtgaagacgacgcggctgatccccctaagagtcaccgaggaggctggagaggacttcacctctgtggtctcagaaatcatgatgtacatccttctggtcttcctcaccttgtggctgctcatcgagatgatatattgctacagaaaggtctcaaaagccgaagaggcagcccaagaaaacgcgtctgactaccttgccatcccatctgagaacaaggagaactctgcggtaccagtggaggaatag

SEQ ID NO: 19 - DNA, 687 bp, Homo sapiens
atgcccggggctggggacggaggcaaagccccggcgagatggctgggcactgggcttttgggcctcttcctgctccccgtaaccctgtcgctggaggtgtctgtgggaaaggccaccgacatctacgctgtcaatggcacggagatcctgctgccctgcaccttctccagctgctttggcttcgaggacctccacttccggtggacctacaacagcagtgacgcattcaagattctcatagaggggactgtgaagaatgagaagtctgaccccaaggtgacgttgaaagacgatgaccgcatcactctggtaggctctactaaggagaagatgaacaacatttccattgtgctgagggacctggagttcagcgacacgggcaaatacacctgccatgtgaagaaccccaaggagaataatctccagcaccacgccaccatcttcctccaagtcgttgatagactggaagaagtggacaacacagtgacactcatcatcctggctgtcgtgggcggggtcatcgggctcctcatcctcatcctgctgatcaagaaactcatcatcttcatcctgaagaagactcgggagaagaagaaggagtgtctcgtgagctcctcggggaatgacaacacggagaacggcttgcctggctccaaggcagaggagaaaccaccttcaaaagtgtga

SEQ ID NO: 20 - protein, 1998 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 21 - protein, 2005 aa, Homo sapiens
MAQSVLVPPGPDSFRFFTRESLAAIEQRIAEEKAKRPKQERKDEDDENGPKPNSDLEAGKSLPFIYGDIPPEMVSVPLEDLDPYYINKKTFIVLNKGKAISRFSATPALYILTPFNPIRKLAIKILVHSLFNMLIMCTILTNCVFMTMSNPPDWTKNVEYTFTGIYTFESLIKILARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCLQWPPDNSSFEINITSFFNNSLDGNGTTFNRTVSIFNWDEYIEDKSHFYFLEGQNDALLCGNSSDAGQCPEGYICVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMLEQLKKQQEEAQAAAAAASAESRDFSGAGGIGVFSESSSVASKLSSKSEKELKNRRKKKKQKEQSGEEEKNDRVRKSESEDSIRRKGFRFSLEGSRLTYEKRFSSPHQSLLSIRGSLFSPRRNSRASLFSFRGRAKDIGSENDFADDEHSTFEDNDSRRDSLFVPHRHGERRHSNVSQASRASRVLPILPMNGKMHSAVDCNGVVSLVGGPSTLTSAGQLLPEGTTTETEIRKRRSSSYHVSMDLLEDPTSRQRAMSIASILTNTMEELEESRQKCPPCWYKFANMCLIWDCCKPWLKVKHLVNLVVMDPFVDLAITICIVLNTLFMAMEHYPMTEQFSSVLSVGNLVFTGIFTAEMFLKIIAMDPYYYFQEGWNIFDGFIVSLSLMELGLANVEGLSVLRSFRLLRVFKLAKSWPTLNMLIKIIGNSVGALGNLTLVLAIIVFIFAVVGMQLFGKSYKECVCKISNDCELPRWHMHDFFHSFLIVFRVLCGEWIETMWDCMEVAGQTMCLTVFMMVMVIGNLVVLNLFLALLLSSFSSDNLAATDDDNEMNNLQIAVGRMQKGIDFVKRKIREFIQKAFVRKQKALDEIKPLEDLNNKKDSCISNHTTIEIGKDLNYLKDGNGTTSGIGSSVEKYVVDESDYMSFINNPSLTVTVPIAVGESDFENLNTEEFSSESDMEESKEKLNATSSSEGSTVDIGAPAEGEQPEVEPEESLEPEACFTEDCVRKFKCCQISIEEGKGKLWWNLRKTCYKIVEHNWFETFIVFMILLSSGALAFEDIYIEQRKTIKTMLEYADKVFTYIFILEMLLKWVAYGFQVYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINYTTGEMFDVSVVNNYSECKALIESNQTARWKNVKVNFDNVGLGYLSLLQVATFKGWMDIMYAAVDSRNVELQPKYEDNLYMYLYFVIFIIFGSFFTLNLFIGVIIDNFNQQKKKFGGQDIFMTEEQKKYYNAMKKLGSKKPQKPIPRPANKFQGMVFDFVTKQVFDISIMILICLNMVTMMVETDDQSQEMTNILYWINLVFIVLFTGECVLKLISLRYYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSGPPDCDPDKDHPGSSVKGDCGNPSVGIFFFVSYIIISFLVVVNMYIAVILENFSVATEESAEPLSEDDFEMFYEVWEKFDPDATQFIEFAKLSDFADALDPPLLIAKPNKVQLIAMDLPMVSGDRIHCLDILFAFTKRVLGESGEMDALRIQMEERFMASNPSKVSYEPITTTLKRKQEEVSAIIIQRAYRRYLLKQKVKKVSSIYKKDKGKECDGTPIKEDTLIDKLNENSTPEKTDMTPSTTSPPSYDSVTKPEKEKFEKDKSEKEDKGKDIRESKK

SEQ ID NO: 22 - protein, 2000 aa, Homo sapiens
MAQALLVPPGPESFRLFTRESLAAIEKRAAEEKAKKPKKEQDNDDENKPKPNSDLEAGKNLPFIYGDIPPEMVSEPLEDLDPYYINKKTFIVMNKGKAIFRFSATSALYILTPLNPVRKIAIKILVHSLFSMLIMCTILTNCVFMTLSNPPDWTKNVEYTFTGIYTFESLIKILARGFCLEDFTFLRDPWNWLDFSVIVMAYVTEFVDLGNVSALRTFRVLRALKTISVIPGLKTIVGALIQSVKKLSDVMILTVFCLSVFALIGLQLFMGNLRNKCLQWPPSDSAFETNTTSYFNGTMDSNGTFVNVTMSTFNWKDYIGDDSHFYVLDGQKDPLLCGNGSDAGQCPEGYICVKAGRNPNYGYTSFDTFSWAFLSLFRLMTQDYWENLYQLTLRAAGKTYMIFFVLVIFLGSFYLVNLILAVVAMAYEEQNQATLEEAEQKEAEFQQMLEQLKKQQEEAQAVAAASAASRDFSGIGGLGELLESSSEASKLSSKSAKEWRNRRKKRRQREHLEGNNKGERDSFPKSESEDSVKRSSFLFSMDGNRLTSDKKFCSPHQSLLSIRGSLFSPRRNSKTSIFSFRGRAKDVGSENDFADDEHSTFEDSESRRDSLFVPHRHGERRNSNVSQASMSSRMVPGLPANGKMHSTVDCNGVVSLVGGPSALTSPTGQLPPEGTTTETEVRKRRLSSYQISMEMLEDSSGRQRAVSIASILTNTMEELEESRQKCPPCWYRFANVFLIWDCCDAWLKVKHLVNLIVMDPFVDLAITICIVLNTLFMAMEHYPMTEQFSSVLTVGNLVFTGIFTAEMVLKIIAMDPYYYFQEGWNIFDGIIVSLSLMELGLSNVEGLSVLRSFRLLRVFKLAKSWPTLNMLIKIIGNSVGALGNLTLVLAIIVFIFAVVGMQLFGKSYKECVCKINDDCTLPRWHMNDFFHSFLIVFRVLCGEWIETMWDCMEVAGQTMCLIVFMLVMVIGNLVVLNLFLALLLSSFSSDNLAATDDDNEMNNLQIAVGRMQKGIDYVKNKMRECFQKAFFRKPKVIEIHEGNKIDSCMSNNTGIEISKELNYLRDGNGTTSGVGTGSSVEKYVIDENDYMSFINNPSLTVTVPIAVGESDFENLNTEEFSSESELEESKEKLNATSSSEGSTVDVVLPREGEQAETEPEEDLKPEACFTEGCIKKFPFCQVSTEEGKGKIWWNLRKTCYSIVEHNWFETFIVFMILLSSGALAFEDIYIEQRKTIKTMLEYADKVFTYIFILEMLLKWVAYGFQTYFTNAWCWLDFLIVDVSLVSLVANALGYSELGAIKSLRTLRALRPLRALSRFEGMRVVVNALVGAIPSIMNVLLVCLIFWLIFSIMGVNLFAGKFYHCVNMTTGNMFDISDVNNLSDCQALGKQARWKNVKVNFDNVGAGYLALLQVATFKGWMDIMYAAVDSRDVKLQPVYEENLYMYLYFVIFIIFGSFFTLNLFIGVIIDNFNQQKKKFGGQDIFMTEEQKKYYNAMKKLGSKKPQKPIPRPANKFQGMVFDFVTRQVFDISIMILICLNMVTMMVETDDQGKYMTLVLSRINLVFIVLFTGEFVLKLVSLRHYYFTIGWNIFDFVVVILSIVGMFLAEMIEKYFVSPTLFRVIRLARIGRILRLIKGAKGIRTLLFALMMSLPALFNIGLLLFLVMFIYAIFGMSNFAYVKKEAGIDDMFNFETFGNSMICLFQITTSAGWDGLLAPILNSAPPDCDPDTIHPGSSVKGDCGNPSVGIFFFVSYIIISFLVVVNMYIAVILENFSVATEESAEPLSEDDFEMFYEVWEKFDPDATQFIEFSKLSDFAAALDPPLLIAKPNKVQLIAMDLPMVSGDRIHCLDILFAFTKRVLGESGEMDALRIQMEDRFMASNPSKVSYEPITTTLKRKQEEVSAAIIQRNFRCYLLKQRLKNISSNYNKEAIKGRIDLPIKQDMIIDKLNGNSTPEKTDGSSSTTSPPSYDSVTKPDKEKFEKDKPEKESKGKEVRENQK

SEQ ID NO: 23 - protein, 1836 aa, Homo sapiens
MARPSLCTLVPLGPECLRPFTRESLAAIEQRAVEEEARLQRNKQMEIEEPERKPRSDLEAGKNLPMIYGDPPPEVIGIPLEDLDPYYSNKKTFIVLNKGKAIFRFSATPALYLLSPFSVVRRGAIKVLIHALFSMFIMITILTNCVFMTMSDPPPWSKNVEYTFTGIYTFESLIKILARGFCVDDFTFLRDPWNWLDFSVIMMAYLTEFVDLGNISALRTFRVLRALKTITVIPGLKTIVGALIQSVKKLSDVMILTVFCLSVFALVGLQLFMGNLRQKCVRWPPPFNDTNTTWYSNDTWYGNDTWYGNEMWYGNDSWYANDTWNSHASWATNDTFDWDAYISDEGNFYFLEGSNDALLCGNSSDAGHCPEGYECIKTGRNPNYGYTSYDTFSWAFLALFRLMTQDYWENLFQLTLRAAGKTYMIFFVVIIFLGSFYLINLILAVVAMAYAEQNEATLAEDKEKEEEFQQMLEKFKKHQEELEKAKAAQALEGGEADGDPAHGKDCNGSLDTSQGEKGAPRQSSSGDSGISDAMEELEEAHQKCPPWWYKCAHKVLIWNCCAPWLKFKNIIHLIVMDPFVDLGITICIVLNTLFMAMEHYPMTEHFDNVLTVGNLVFTGIFTAEMVLKLIAMDPYEYFQQGWNIFDSIIVTLSLVELGLANVQGLSVLRSFRLLRVFKLAKSWPTLNMLIKIIGNSVGALGNLTLVLAIIVFIFAVVGMQLFGKSYKECVCKIALDCNLPRWHMHDFFHSFLIVFRILCGEWIETMWDCMEVAGQAMCLTVFLMVMVIGNLVVLNLFLALLLSSFSADSLAASDEDGEMNNLQIAIGRIKLGIGFAKAFLLGLLHGKILSPKDIMLSLGEADGAGEAGEAGETAPEDEKKEPPEEDLKKDNHILNHMGLADGPPSSLELDHLNFINNPYLTIQVPIASEESDLEMPTEEETDTFSEPEDSKKPPQPLYDGNSSVCSTADYKPPEEDPEEQAEENPEGEQPEECFTEACVQRWPCLYVDISQGRGKKWWTLRRACFKIVEHNWFETFIVFMILLSSGALAFEDIYIEQRRVIRTILEYADKVFTYIFIMEMLLKWVAYGFKVYFTNAWCWLDFLIVDVSIISLVANWLGYSELGPIKSLRTLRALRPLRALSRFEGMRVVVNALLGAIPSIMNVLLVCLIFWLIFSIMGVNLFAGKFYYCINTTTSERFDISEVNNKSECESLMHTGQVRWLNVKVNYDNVGLGYLSLLQVATFKGWMDIMYAAVDSREKEEQPQYEVNLYMYLYFVIFIIFGSFFTLNLFIGVIIDNFNQQKKKLGGKDIFMTEEQKKYYNAMKKLGSKKPQKPIPRPQNKIQGMVYDLVTKQAFDITIMILICLNMVTMMVETDNQSQLKVDILYNINMIFIIIFTGECVLKMLALRQYYFTVGWNIFDFVVVILSIVGLALSDLIQKYFVSPTLFRVIRLARIGRVLRLIRGAKGIRTLLFALMMSLPALFNIGLLLFLVMFIYSIFGMSNFAYVKKESGIDDMFNFETFGNSIICLFEITTSAGWDGLLNPILNSGPPDCDPNLENPGTSVKGDCGNPSIGICFFCSYIIISFLIVVNMYIAIILENFNVATEESSEPLGEDDFEMFYETWEKFDPDATQFIAYSRLSDFVDTLQEPLRIAKPNKIKLITLDLPMVPGDKIHCLDILFALTKEVLGDSGEMDALKQTMEEKFMAANPSKVSYEPITTTLKRKHEEVCAIKIQRAYRRHLLQRSMKQASYMYRHSHDGSGDDAPEKEGLLANTMSKMYGHENGNSSSPSPEEKGEAGDAGPTMGLMPISPSDTAWPPAPPPGQTVRPGVKESLV

SEQ ID NO: 24 - protein, 2016 aa, Homo sapiens [sequence omitted]

SEQ ID NO: 25 - protein, 1682 aa, Homo sapiens
MLASPEPKGLVPFTKESFELIKQHIAKTHNEDHEEEDLKPTPDLEVGKKLPFIYGNLSQGMVSEPLEDVDPYYYKKKNTFIVLNKNRTIFRFNAASILCTLSPFNCIRRTTIKVLVHPFFQLFILISVLIDCVFMSLTNLPKWRPVLENTLLGIYTFEILVKLFARGVWAGSFSFLGDPWNWLDFSVTVFEVIIRYSPLDFIPTLQTARTLRILKIIPLNQGLKSLVGVLIHCLKQLIGVIILTLFFLSIFSLIGMGLFMGNLKHKCFRWPQENENETLHNRTGNPYYIRETENFYYLEGERYALLCGNRTDAGQCPEGYVCVKAGINPDQGFTNFDSFGWALFALFRLMAQDYPEVLYHQILYASGKVYMIFFVVVSFLFSFYMASLFLGILAMAYEEEKQRVGEISKKIEPKFQQTGKELQEGNETDEAKTIQIEMKKRSPISTDTSLDVLEDATLRHKEELEKSKKICPLYWYKFAKTFLIWNCSPCWLKLKEFVHRIIMAPFTDLFLIICIILNVCFLTLEHYPMSKQTNTLLNIGNLVFIGIFTAEMIFKIIAMHPYGYFQVGWNIFDSMIVFHGLIELCLANVAGMALLRLFRMLRIFKLGKYWPTFQILMWSLSNSWVALKDLVLLLFTFIFFSAAFGMKLFGKNYEEFVCHIDKDCQLPRWHMHDFFHSFLNVFRILCGEWVETLWDCMEVAGQSWCIPFYLMVILIGNLLVLYLFLALVSSFSSCKDVTAEENNEAKNLQLAVARIKKGINYVLLKILCKTQNVPKDTMDHVNEVYVKEDISDHTLSELSNTQDFLKDKEKSSGTEKNATENESQSLIPSPSVSETVPIASGESDIENLDNKEIQSKSGDGGSKEKIKQSSSSECSTVDIAISEEEEMFYGGERSKHLKNGCRRGSSLGQISGASKKGKIWQNIRKTCCKIVENNWFKCFIGLVTLLSTGTLAFEDIYMDQRKTIKILLEYADMIFTYIFILEMLLKWMAYGFKAYFSNGWYRLDFVVVIVFCLSLIGKTREELKPLISMKFLRPLRVLSQFERMKVVVRALIKTTLPTLNVFLVCLMIWLIFSIMGVDLFAGRFYECIDPTSGERFPSSEVMNKSRCESLLFNESMLWENAKMNFDNVGNGFLSLLQVATFNGWITIMNSAIDSVAVNIQPHFEVNIYMYCYFINFIIFGVFLPLSMLITVIIDNFNKHKIKLGGSNIFITVKQRKQYRRLKKLMYEDSQRPVPRPLNKLQGFIFDVVTSQAFNVIVMVLICFQAIAMMIDTDVQSLQMSIALYWINSIFVMLYTMECILKLIAFRCFYFTIAWNIFDFMVVIFSITGLCLPMTVGSYLVPPSLVQLILLSRIIHMLRLGKGPKVFHNLMLPLMLSLPALLNIILLIFLVMFIYAVFGMYNFAYVKKEAGINDVSNFETFGNSMLCLFQVAIFAGWDGMLDAIFNSKWSDCDPDKINPGTQVRGDCGNPSVGIFYFVSYILISWLIIVNMYIVVVMEFLNIASKKKNKTLSEDDFRKFFQVWKRFDPDRTQYIDSSKLSDFAAALDPPLFMAKPNKGQLIALDLPMAVGDRIHCLDILLAFTKRVMGQDVRMEKVVSEIESGFLLANPFKITCEPITTTLKRKQEAVSATIIQRAYKNYRLRRNDKNTSDIHMIDGDRDVHATKEGAYFDKAKEKSPIQSQI

SEQ ID NO: 26 - protein, 1980 aa, Homo sapiens
MAARLLAPPGPDSFKPFTPESLANIERRIAESKLKKPPKADGSHREDDEDSKPKPNSDLEAGKSLPFIYGDIPQGLVAVPLEDFDPYYLTQKTFVVLNRGKTLFRFSATPALYILSPFNLIRRIAIKILIHSVFSMIIMCTILTNCVFMTFSNPPDWSKNVEYTFTGIYTFESLVKIIARGFCIDGFTFLRDPWNWLDFSVIMMAYITEFVNLGNVSALRTFRVLRALKTISVIPGLKTIVGALIQSVKKLSDVMILTVFCLSVFALIGLQLFMGNLRNKCVVWPINFNESYLENGTKGFDWEEYINNKTNFYTVPGMLEPLLCGNSSDAGQCPEGYQCMKAGRNPNYGYTSFDTFSWAFLALFRLMTQDYWENLYQLTLRAAGKTYMIFFVLVIFVGSFYLVNLILAVVAMAYEEQNQATLEEAEQKEAEFKAMLEQLKKQQEEAQAAAMATSAGTVSEDAIEEEGEEGGGSPRSSSEISKLSSKSAKERRNRRKKRKQKELSEGEEKGDPEKVFKSESEDGMRRKAFRLPDNRIGRKFSIMNQSLLSIPGSPFLSRHNSKSSIFSFRGPGRFRDPGSENEFADDEHSTVEESEGRRDSLFIPIRARERRSSYSGYSGYSQGSRSSRIFPSLRRSVKRNSTVDCNGVVSLIGGPGSHIGGRLLPEATTEVEIKKKGPGSLLVSMDQLASYGRKDRINSIMSVVTNTLVEELEESQRKCPPCWYKFANTFLIWECHPYWIKLKEIVNLIVMDPFVDLAITICIVLNTLFMAMEHHPMTPQFEHVLAVGNLVFTGIFTAEMFLKLIAMDPYYYFQEGWNIFDGFIVSLSLMELSLADVEGLSVLRSFRLLRVFKLAKSWPTLNMLIKIIGNSVGALGNLTLVLAIIVFIFAVVGMQLFGKSYKECVCKINQDCELPRWHMHDFFHSFLIVFRVLCGEWIETMWDCMEVAGQAMCLIVFMMVMVIGNLVVLNLFLALLLSSFSADNLAATDDDGEMNNLQISVIRIKKGVAWTKLKVHAFMQAHFKQREADEVKPLDELYEKKANCIANHTGADIHRNGDFQKNGNGTTSGIGSSVEKYIIDEDHMSFINNPNLTVRVPIAVGESDFENLNTEDVSSESDPEGSKDKLDDTSSSEGSTIDIKPEVEEVPVEQPEEYLDPDACFTEGCVQRFKCCQVNIEEGLGKSWWILRKTCFLIVEHNWFETFIIFMILLSSGALAFEDIYIEQRKTIRTILEYADKVFTYIFILEMLLKWTAYGFVKFFTNAWCWLDFLIVAVSLVSLIANALGYSELGAIKSLRTLRALRPLRALSRFEGMRVVVNALVGAIPSIMNVLLVCLIFWLIFSIMGVNLFAGKYHYCFNETSEIRFEIEDVNNKTECEKLMEGNNTEIRWKNVKINFDNVGAGYLALLQVATFKGWMDIMYAAVDSRKPDEQPKYEDNIYMYIYFVIFIIFGSFFTLNLFIGVIIDNFNQQKKKFGGQDIFMTEEQKKYYNAMKKLGSKKPQKPIPRPLNKIQGIVFDFVTQQAFDIVIMMLICLNMVTMMVETDTQSKQMENILYWINLVFVIFFTCECVLKMFALRHYYFTIGWNIFDFVVVILSIVGMFLADIIEKYFVSPTLFRVIRLARIGRILRLIKGAKGIRTLLFALMMSLPALFNIGLLLFLVMFIFSIFGMSNFAYVKHEAGIDDMFNFETFGNSMICLFQITTSAGWDGLLLPILNRPPDCSLDKEHPGSGFKGDCGNPSVGIFFFVSYIIISFLIVVNMYIAIILENFSVATEESADPLSEDDFETFYEIWEKFDPDATQFIEYCKLADFADALEHPLRVPKPNTIELIAMDLPMVSGDRIHCLDILFAFTKRVLGDSGELDILRQQMEERFVASNPSKVSYEPITTTLRRKQEEVSAVVLQRAYRGHLARRGFICKKTTSNKLENGGTHREKKESTPSTASLPSYDSVTKPEKEKQQRAEEGRRERAKRQKEVRESKC

SEQ ID NO: 27 - protein, 1977 aa, Homo sapiens
MAMLPPPGPQSFVHFTKQSLALIEQRIAERKSKEPKEEKKDDDEEAPKPSSDLEAGKQLPFIYGDIPPGMVSEPLEDLDPYYADKKTFIVLNKGKTIFRFNATPALYMLSPFSPLRRISIKILVHSLFSMLIMCTILTNCIFMTMNNPPDWTKNVEYTFTGIYTFESLVKILARGFCVGEFTFLRDPWNWLDFVVIVFAYLTEFVNLGNVSALRTFRVLRALKTISVIPGLKTIVGALIQSVKKLSDVMILTVFCLSVFALIGLQLFMGNLKHKCFRNSLENNETLESIMNTLESEEDFRKYFYYLEGSKDALLCGFSTDSGQCPEGYTCVKIGRNPDYGYTSFDTFSWAFLALFRLMTQDYWENLYQQTLRAAGKTYMIFFVVVIFLGSFYLINLILAVVAMAYEEQNQANIEEAKQKELEFQQMLDRLKKEQEEAEAIAAAAAEYTSIRRSRIMGLSESSSETSKLSSKSAKERRNRRKKKNQKKLSSGEEKGDAEKLSKSESEDSIRRKSFHLGVEGHRRAHEKRLSTPNQSPLSIRGSLFSARRSSRTSLFSFKGRGRDIGSETEFADDEHSIFGDNESRRGSLFVPHRPQERRSSNISQASRSPPMLPVNGKMHSAVDCNGVVSLVDGRSALMLPNGQLLPEGTTNQIHKKRRCSSYLLSEDMLNDPNLRQRAMSRASILTNTVEELEESRQKCPPWWYRFAHKFLIWNCSPYWIKFKKCIYFIVMDPFVDLAITICIVLNTLFMAMEHHPMTEEFKNVLAIGNLVFTGIFAAEMVLKLIAMDPYEYFQVGWNIFDSLIVTLSLVELFLADVEGLSVLRSFRLLRVFKLAKSWPTLNMLIKIIGNSVGALGNLTLVLAIIVFIFAVVGMQLFGKSYKECVCKINDDCTLPRWHMNDFFHSFLIVFRVLCGEWIETMWDCMEVAGQAMCLIVYMMVMVIGNLVVLNLFLALLLSSFSSDNLTAIEEDPDANNLQIAVTRIKKGINYVKQTLREFILKAFSKKPKISREIRQAEDLNTKKENYISNHTLAEMSKGHNFLKEKDKISGFGSSVDKHLMEDSDGQSFIHNPSLTVTVPIAPGESDLENMNAEELSSDSDSEYSKVRLNRSSSSECSTVDNPLPGEGEEAEAEPMNSDEPEACFTDGCVRRFSCCQVNIESGKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMRVVVNALIGAIPSIMNVLLVCLIFWLIFSIMGVNLFAGKFYECINTTDGSRFPASQVPNRSECFALMNVSQNVRWKNLKVNFDNVGLGYLSLLQVATFKGWTIIMYAAVDSVNVDKQPKYEYSLYMYIYFVVFIIFGSFFTLNLFIGVIIDNFNQQKKKLGGQDIFMTEEQKKYYNAMKKLGSKKPQKPIPRPGNKIQGCIFDLVTNQAFDISIMVLICLNMVTMMVEKEGQSQHMTEVLYWINVVFIILFTGECVLKLISLRHYYFTVGWNIFDFVVVIISIVGMFLADLIETYFVSPTLFRVIRLARIGRILRLVKGAKGIRTLLFALMMSLPALFNIGLLLFLVMFIYAIFGMSNFAYVKKEDGINDMFNFETFGNSMICLFQITTSAGWDGLLAPILNSKPPDCDPKKVHPGSSVEGDCGNPSVGIFYFVSYIIISFLVVVNMYIAVILENFSVATEESTEPLSEDDFEMFYEVWEKFDPDATQFIEFSKLSDFAAALDPPLLIAKPNKVQLIAMDLPMVSGDRIHCLDILFAFTKRVLGESGEMDSLRSQMEERFMSANPSKVSYEPITTTLKRKQEDVSATVIQRAYRRYRLRQNVKNISSIYIKDGDRDDDLLNKKDMAFDNVNENSSPEKTDATSSTTSPPSYDSVTKPDKEKYEQDRTEKEDKGKDSKESKK

SEQ ID NO: 28 - protein, 1956 aa, Homo sapiens
MEFPIGSLETNNFRRFTPESLVEIEKQIAAKQGTKKAREKHREQKDQEEKPRPQLDLKACNQLPKFYGELPAELIGEPLEDLDPFYSTHRTFMVLNKGRTISRFSATRALWLFSPFNLIRRTAIKVSVHSWFSLFITVTILVNCVCMTRTDLPEKIEYVFTVIYTFEALIKILARGFCLNEFTYLRDPWNWLDFSVITLAYVGTAIDLRGISGLRTFRVLRALKTVSVIPGLKVIVGALIHSVKKLADVTILTIFCLSVFALVGLQLFKGNLKNKCVKNDMAVNETTNYSSHRKPDIYINKRGTSDPLLCGNGSDSGHCPDGYICLKTSDNPDFNYTSFDSFAWAFLSLFRLMTQDSWERLYQQTLRTSGKIYMIFFVLVIFLGSFYLVNLILAVVTMAYEEQNQATTDEIEAKEKKFQEALEMLRKEQEVLAALGIDTTSLHSHNGSPLTSKNASERRHRIKPRVSEGSTEDNKSPRSDPYNQRRMSFLGLASGKRRASHGSVFHFRSPGRDISLPEGVTDDGVFPGDHESHRGSLLLGGGAGQQGPLPRSPLPQPSNPDSRHGEDEHQPPPTSELAPGAVDVSAFDAGQKKTFLSAEYLDEPFRAQRAMSVVSIITSVLEELEESEQKCPPCLTSLSQKYLIWDCCPMWVKLKTILFGLVTDPFAELTITLCIVVNTIFMAMEHHGMSPTFEAMLQIGNIVFTIFFTAEMVFKIIAFDPYYYFQKKWNIFDCIIVTVSLLELGVAKKGSLSVLRSFRLLRVFKLAKSWPTLNTLIKIIGNSVGALGNLTIILAIIVFVFALVGKQLLGENYRNNRKNISAPHEDWPRWHMHDFFHSFLIVFRILCGEWIENMWACMEVGQKSICLILFLTVMVLGNLVVLNLFIALLLNSFSADNLTAPEDDGEVNNLQVALARIQVFGHRTKQALCSFFSRSCPFPQPKAEPELVVKLPLSSSKAENHIAANTARGSSGGLQAPRGPRDEHSDFIANPTVWVSVPIAEGESDLDDLEDDGGEDAQSFQQEVIPKGQQEQLQQVERCGDHLTPRSPGTGTSSEDLAPSLGETWKDESVPQVPAEGVDDTSSSEGSTVDCLDPEEILRKIPELADDLEEPDDCFTEGCIRHCPCCKLDTTKSPWDVGWQVRKTCYRIVEHSWFESFIIFMILLSSGSLAFEDYYLDQKPTVKALLEYTDRVFTFIFVFEMLLKWVAYGFKKYFTNAWCWLDFLIVNISLISLTAKILEYSEVAPIKALRTLRALRPLRALSRFEGMRVVVDALVGAIPSIMNVLLVCLIFWLIFSIMGVNLFAGKFWRCINYTDGEFSLVPLSIVNNKSDCKIQNSTGSFFWVNVKVNFDNVAMGYLALLQVATFKGWMDIMYAAVDSREVNMQPKWEDNVYMYLYFVIFIIFGGFFTLNLFVGVIIDNFNQQKKKLGGQDIFMTEEQKKYYNAMKKLGSKKPQKPIPRPLNKFQGFVFDIVTRQAFDITIMVLICLNMITMMVETDDQSEEKTKILGKINQFFVAVFTGECVMKMFALRQYYFTNGWNVFDFIVVVLSIASLIFSAILKSLQSYFSPTLFRVIRLARIGRILRLIRAAKGIRTLLFALMMSLPALFNIGLLLFLVMFIYSIFGMSSFPHVRWEAGIDDMFNFQTFANSMLCLFQITTSAGWDGLLSPILNTGPPYCDPNLPNSNGTRGDCGSPAVGIIFFTTYIIISFLIMVNMYIAVILENFNVATEESTEPLSEDDFDMFYETWEKFDPEATQFITFSALSDFADTLSGPLRIPKPNRNILIQMDLPLVPGDKIHCLDILFAFTKNVLGESGELDSLKANMEEKFMATNLSKSSYEPIATTLRWKQEDISATVIQKAYRSYVLHRSMALSNTPCVPRAEEEAASLPDEGFVAFTANENCVLPDKSETASATSFPPSYESVTRGLSDRVNMRTSSSIQNEDEATSMELIAPGP

SEQ ID NO: 29 - protein, 1791 aa, Homo sapiens
MDDRCYPVIFPDERNFRPFTSDSLAAIEKRIAIQKEKKKSKDQTGEVPQPRPQLDLKASRKLPKLYGDIPRELIGKPLEDLDPFYRNHKTFMVLNRKRTIYRFSAKHALFIFGPFNSIRSLAIRVSVHSLFSMFIIGTVIINCVFMATGPAKNSNSNNTDIAECVFTGIYIFEALIKILARGFILDEFSFLRDPWNWLDSIVIGIAIVSYIPGITIKLLPLRTFRVFRALKAISVVSRLKVIVGALLRSVKKLVNVIILTFFCLSIFALVGQQLFMGSLNLKCISRDCKNISNPEAYDHCFEKKENSPEFKMCGIWMGNSACSIQYECKHTKINPDYNYTNFDNFGWSFLAMFRLMTQDSWEKLYQQTLRTTGLYSVFFFIVVIFLGSFYLINLTLAVVTMAYEEQNKNVAAEIEAKEKMFQEAQQLLKEEKEALVAMGIDRSSLTSLETSYFTPKKRKLFGNKKRKSFFLRESGKDQPPGSDSDEDCQKKPQLLEQTKRLSQNLSLDHFDEHGDPLQRQRALSAVSILTITMKEQEKSQEPCLPCGENLASKYLVWNCCPQWLCVKKVLRTVMTDPFTELAITICIIINTVFLAMEHHKMEASFEKMLNIGNLVFTSIFIAEMCLKIIALDPYHYFRRGWNIFDSIVALLSFADVMNCVLQKRSWPFLRSFRVLRVFKLAKSWPTLNTLIKIIGNSVGALGSLTVVLVIVIFIFSVVGMQLFGRSFNSQKSPKLCNPTGPTVSCLRHWHMGDFWHSFLVVFRILCGEWIENMWECMQEANASSSLCVIVFILITVIGKLVVLNLFIALLLNSFSNEERNGNLEGEARKTKVQLALDRFRRAFCFVRHTLEHFCHKWCRKQNLPQQKEVAGGCAAQSKDIIPLVMEMKRGSETQEELGILTSVPKTLGVRHDWTWLAPLAEEEDDVEFSGEDNAQRITQPEPEQQAYELHQENKKPTSQRVQSVEIDMFSEDEPHLTIQDPRKKSDVTSILSECSTIDLQDGFGWLPEMVPKKQPERCLPKGFGCCFPCCSVDKRKPPWVIWWNLRKTCYQIVKHSWFESFIIFVILLSSGALIFEDVHLENQPKIQELLNCTDIIFTHIFILEMVLKWVAFGFGKYFTSAWCCLDFIIVIVSVTTLINLMELKSFRTLRALRPLRALSQFEGMKVVVNALIGAIPAILNVLLVCLIFWLVFCILGVYFFSGKFGKCINGTDSVINYTIITNKSQCESGNFSWINQKVNFDNVGNAYLALLQVATFKGWMDIIYAAVDSTEKEQQPEFESNSLGYIYFVVFIIFGSFFTLNLFIGVIIDNFNQQQKKLGGQDIFMTEEQKKYYNAMKKLGSKKPQKPIPRPLNKCQGLVFDIVTSQIFDIIIISLIILNMISMMAESYNQPKAMKSILDHLNWVFVVIFTLECLIKIFALRQYYFTNGWNLFDCVVVLLSIVSTMISTLENQEHIPFPPTLFRIVRLARIGRILRLVRAARGIRTLLFALMMSLPSLFNIGLLLFLIMFIYAILGMNWFSKVNPESGIDDIFNFKTFASSMLCLFQISTSAGWDSLLSPMLRSKESCNSSSENCHLPGIATSYFVSYIIISFLIVVNMYIAVILENFNTATEESEDPLGEDDFDIFYEVWEKFDPEATQFIKYSALSDFADALPEPLRVAKPNKYQFLVMDLPMVSEDRLHCMDILFAFTARVLGGSDGLDSMKAMMEEKFMEANPLKKLYEPIVTTTKRKEEERGAAIIQKAFRKYMMKVTKGDQGDQNDLENGPHSPLQTLCNGDLSSFGVAKGKVHCD

SEQ ID NO: 30 - protein, 218 aa, Homo sapiens
MGRLLALVVGAALVSSACGGCVEVDSETEAVYGMTFKILCISCKRRSETNAETFTEWTFRQKGTEEFVKILRYENEVLQLEEDERFEGRVVWNGSRGTKDLQDLSIFITNVTYNHSGDYECHVYRLLFFENYEHNTSVVKKIHIEVVDKANRDMASIVSEIMMYVLIVVLTIWLVAEMIYCYKKIAAATETAAQENASEYLAITSESKENCTGVQVAE

SEQ ID NO: 31 - protein, 215 aa, Homo sapiens
MHRDAWLPRPAFSLTGLSLFFSLVPPGRSMEVTVPATLNVLNGSDARLPCTFNSCYTVNHKQFSLNWTYQECNNCSEEMFLQFRMKIINLKLERFQDRVEFSGNPSKYDVSVMLRNVQPEDEGIYNCYIMNPPDRHRGHGKIHLQVLMEEPPERDSTVAVIVGASVGGFLAVVILVLMVVKCVRRKKEQKLSTDDLKTEEEGKTDGEGNPDDGAK

SEQ ID NO: 32 - protein, 215 aa, Homo sapiens
MPAFNRLFPLASLVLIYWVSVCFPVCVEVPSETEAVQGNPMKLRCISCMKREEVEATTVVEWFYRPEGGKDFLIYEYRNGHQEVESPFQGRLQWNGSKDLQDVSITVLNVTLNDSGLYTCNVSREFEFEAHRPFVKTTRLIPLRVTEEAGEDFTSVVSEIMMYILLVFLTLWLLIEMIYCYRKVSKAEEAAQENASDYLAIPSENKENSAVPVEE

SEQ ID NO: 33 - protein, 228 aa, Homo sapiens
MPGAGDGGKAPARWLGTGLLGLFLLPVTLSLEVSVGKATDIYAVNGTEILLPCTFSSCFGFEDLHFRWTYNSSDAFKILIEGTVKNEKSDPKVTLKDDDRITLVGSTKEKMNNISIVLRDLEFSDTGKYTCHVKNPKENNLQHHATIFLQVVDRLEEVDNTVTLIILAVVGGVIGLLILILLIKKLIIFILKKTREKKKECLVSSSGNDNTENGLPGSKAEEKPPSKV

SEQ ID NO: 34 - DNA, 34 bp, Artificial Sequence
  source: /note=“Description of Artificial Sequence Synthetic probe”
gcgagagcgacaagcagaccctatagaacctcgc

SEQ ID NO: 35 - protein, 6 aa, Artificial Sequence
  source: /note=“Description of Artificial Sequence Synthetic 6xHis tag”
HHHHHH

(19) United States
(12) Patent Application Publication
(10) Pub. No.: US 2017/0219606 A1
(43) Pub. Date: Aug. 3, 2017

(54) CELL LINES EXPRESSING NaV AND METHODS OF USING THEM

(71) Applicant: Chromocell Corporation, North Brunswick, NJ (US)

(72) Inventors: Kambiz Shekdar, New York, NY (US); Olga Dedova, East Brunswick, NJ (US)

(21) Appl. No.: 15/000,974

(22) Filed: Jan. 19, 2016

Related U.S. Application Data
(63) Continuation of application No. 13/147,333, filed on Aug. 1, 2011, now abandoned.

(51) Int. Cl. G01N 33/68; C07K 14/705; G01N 33/50

(52) U.S. Cl. CPC G01N 33/6872; G01N 33/502; C07K 14/705; G01N 2500/10

(57) ABSTRACT

Cells and cell lines that express voltage-gated sodium ion channels (NaV) and methods for using the cells and cell lines are disclosed herein. The NaV-expressing cells and cell lines are useful in cell-based assays, e.g., high throughput screening assays.

### BACKGROUND

The voltage-gated sodium ion channel family referred to as the NaV family are large and complex molecules that are expressed in the central nervous system, including the brain, in the peripheral nervous system and in muscle, including cardiac muscle. All of the family members are important clinical targets for managing a variety of conditions including epilepsy, muscle paralysis and pain. NaV channels are cell membrane embedded proteins comprising an alpha subunit and one or more beta subunits. Genes coding for ten alpha subunits and four beta subunits have been identified (see, e.g., Catterall et al., Pharmacol Rev. 55:575-578 (2003); Isom, Neuroscientist, 7:42-54 (2001)). The alpha subunit forms the ion pore and is thought to be responsible for selective sodium conduction and voltage-dependent activation and inactivation (see, e.g., Liu et al., Assay Drug Dev Tech, 4(1):37-48 (2006)). Beta subunits have been shown to modify expression levels and biophysical characteristics of some alpha subunits. Liu et al., supra. Both the alpha and beta subunits are differentially expressed in different tissues. Id.

The discovery of new and improved therapeutics that specifically target NaV family members has been hampered by the lack of cell-based systems and especially cell-based systems that are amenable to high throughput formats for identifying and testing NaV modulators. Cell-based systems are preferred for drug discovery and validation because they provide a functional assay for a compound as opposed to cell-free systems, which only provide a binding assay. Moreover, cell-based systems have the advantage of simultaneously testing cytotoxicity. The present invention addresses this need.

### SUMMARY OF THE INVENTION

We have discovered new and useful cells and cell lines that express various forms of NaV, including functional NaV and various combinations of subunits of NaV. These cells and cell lines are useful in cell-based assays, in particular high throughput assays to study the functions of NaV and to screen for NaV modulators.

Accordingly, the invention provides a cell or cell line engineered (altered) to stably express a NaV, which cell or cell line produces a Z′ factor of at least 0.5 in an assay. In some embodiments, the Z′ factor can be at least 0.55, 0.60, 0.65, 0.70, 0.75, 0.80, or 0.85. The cells and cell lines of the invention may be grown (e.g., maintained) in culture in the absence of selective pressure, and may continue to express the NaV for at least 15 days, 30 days, 45 days, 60 days, 75 days, 100 days, 120 days, or 150 days despite the absence of selective pressure. In some embodiments, the cells or cell lines growing in the absence of selective pressure express NaV at a consistent level for at least 15 days, 30 days, 45 days, 60 days, 75 days, 100 days, 120 days, or 150 days.

In some embodiments, the NaV expressed in the present cells and cell lines comprises an alpha subunit and a beta subunit, and may optionally further comprise a different beta subunit. In some embodiments, the NaV comprises at least one subunit that is expressed from an introduced nucleic acid encoding it, and/or comprises at least one NaV subunit that is expressed from an endogenous gene activated by gene activation. In some embodiments, the NaV is native, e.g., containing no polypeptide tag.

The cells or cell lines of the invention may be eukaryotic cells (e.g., mammalian cells), and optionally do not express NAV endogenously (or in the case of gene activation, do not express NAV endogenously prior to gene activation). The cells may be primary or immortalized cells, and may be cells of, for example, primate (e.g., human or monkey), rodent (e.g., mouse, rat, or hamster), or insect (e.g., fruit fly) origin.

The NaV expressed in the present cells and cell lines may comprise two, three, or four subunits. The NaV may be a human NaV. The NaV may comprise an alpha 1, alpha 2, alpha 3, alpha 4, alpha 5, alpha 7, alpha 8, alpha 9, alpha 10, or alpha 11 subunit. The NaV may comprise one, two, or more beta subunits independently selected from the group consisting of a beta 1 subunit, a beta 2 subunit, a beta 3 subunit, or a beta 4 subunit. When the NaV has more than one beta subunit, the beta subunits may be the same or different. The subunits in a NaV protein may be from the same or different species.

In further embodiments, the NaV alpha subunit is selected from the group consisting of:
an alpha 1 subunit having the amino acid sequence set forth in SEQ ID NO:20;
an alpha 2 subunit having the amino acid sequence set forth in SEQ ID NO:21;
an alpha 3 subunit having the amino acid sequence set forth in SEQ ID NO:22;
an alpha 4 subunit having the amino acid sequence set forth in SEQ ID NO:23;
an alpha 5 subunit having the amino acid sequence set forth in SEQ ID NO:24;
an alpha 7 subunit having the amino acid sequence set forth in SEQ ID NO:25;
an alpha 8 subunit having the amino acid sequence set forth in SEQ ID NO:26;
an alpha 9 subunit having the amino acid sequence set forth in SEQ ID NO:27;
an alpha 10 subunit having the amino acid sequence set forth in SEQ ID NO:28;
an alpha 11 subunit having the amino acid sequence set forth in SEQ ID NO:29;
a polypeptide with at least 95% sequence identity, or substantially identical, to any one of SEQ ID NOS:20-29, where the polypeptide may form a voltage-gated ion channel; and
a polypeptide that is an allelic variant to any one of SEQ ID NOS:20-29.

In further embodiments, the NaV alpha subunit is encoded by a nucleic acid sequence selected from the group consisting of SEQ ID NOS:6-15; a nucleic acid sequence that hybridizes under stringent conditions to any one of SEQ ID NOS:6-15; a nucleic acid sequence with at least 95% sequence identity, or substantially identical, to any one of SEQ ID NOS:6-15 and a nucleic acid sequence that is an allelic variant of any one of SEQ ID NOS:6-15.

In some embodiments, the NaV beta subunit is selected from the group consisting of:
a beta 1 subunit having the amino acid sequence set forth in SEQ ID NO:30;
a beta 2 subunit having the amino acid sequence set forth in SEQ ID NO:31;
a beta 3 subunit having the amino acid sequence set forth in SEQ ID NO:32;
a beta 4 subunit having the amino acid sequence set forth in SEQ ID NO:33;
a polypeptide with at least 95% sequence identity, or substantially identical, to any one of SEQ ID NOS:30-33, wherein the polypeptide may modulate a voltage-gated ion channel; and
a polypeptide that is an allelic variant to any one of SEQ ID NOS:30-33.

In further embodiments, the beta subunit is encoded by a nucleic acid sequence individually selected from the group consisting of: SEQ ID NOS:16-19; a nucleic acid that hybridizes under stringent conditions to any one of SEQ ID NOS:16-19; a nucleic acid with at least 95% sequence identity, or substantially identical, to any one of SEQ ID NOS:16-19; and a nucleotide that is an allelic variant of any one

An example of NaV may comprise a human NaV alpha 9 subunit, a human beta 1 subunit, and a human beta 2 subunit. The human alpha 9 subunit may comprise (1) the amino acid sequence set forth in SEQ ID NO:27; or (2) an amino acid sequence encoded by a nucleic acid sequence set forth in SEQ ID NO:13. The human beta 1 subunit may comprise (1) the amino acid sequence set forth in SEQ ID NO: 30, or (2) an amino acid sequence encoded by a nucleic acid sequence set forth in SEQ ID NO:16. The human beta 2 subunit may comprise (1) the amino acid sequence set forth in SEQ ID NO:31, or (2) an amino acid sequence encoded by a nucleic acid sequence set forth in SEQ ID NO:17. In some embodiments, the native NaV may comprise a polypeptide comprising an amino acid sequence set forth in SEQ ID NO:27; a polypeptide comprising the amino acid sequence set forth in SEQ ID NO:30; and a polypeptide comprising the amino acid sequence set forth in SEQ ID NO:31.

The invention also provides a collection of the cells or cell lines of the invention, wherein the cells or cell lines in the collection express different or the same forms of NaV. The collection may also comprise cells expressing a control protein. In some embodiments, the cells or cell lines in a collection are matched to share physiological properties (e.g., cell type, metabolism, cell passage (age), growth rate, adherence to a tissue culture surface, Z′ factor, expression level of NaV) to allow parallel processing and accurate assay readouts. The matching can be achieved by, for example, generating and growing the cells and cell lines under identical conditions, achievable by, e.g., automation.

The invention further provides a method for identifying a NaV modulator, comprising the steps of contacting a cell, a cell line, or a cell (line) collection of the invention with a test compound; and detecting a change in a NaV function in a cell compared to a cell not contacted with the test compound, wherein a change in said function indicates that the test compound is a NaV modulator. The test compound may be a small molecule, a polypeptide, a peptide, or an antibody or an antigen-binding portion thereof. The test compound may be in a library of compounds. The library may be a small molecule library, a combinatorial library, a peptide library or an antibody library. In the present method, the detecting step may be selected from a membrane potential assay, an electrophysiology assay, a binding assay, and the like, and the method may be implemented in a high throughout manner.

The invention also provides a cell engineered to stably express a NaV at a consistent level over time, the cell made by a method comprising the steps of: (a) providing a plurality of cells that express mRNA(s) encoding the NaV; (b) dispersing the cells individually into individual culture vessels, thereby providing a plurality of separate cell cultures; (c) culturing the cells under a set of desired culture conditions using automated cell culture methods characterized in that the conditions are substantially identical for each of the separate cell cultures, during which culturing the number of cells per separate cell culture is normalized, and wherein the separate cultures are passaged on the same schedule; (d) assaying the separate cell cultures to measure expression of the NaV at least twice; and (e) identifying a separate cell culture that expresses the NaV at a consistent level in both assays, thereby obtaining said cell.

### BRIEF DESCRIPTION OF THE FIGURES

FIG. 1 is a bar graph depicting relative expression of the heterologous human NaV 1.7 α, β1, and β2 subunits in stable NaV 1.7-expressing cell lines. The expression levels were assayed by quantitative RT-PCR and normalized to the expression level of a control GAPDH gene. (+) lanes indicate reactions with reverse transcriptase enzyme added and (−) lanes indicate reactions without reverse transcriptase enzyme.

FIG. 2 shows the regulation of NaV 1.7 α subunit expression by auxiliary β subunits. Comparative RT-PCR illustrated increased detection of α subunit expression in drug-selected cells when all three NaV 1.7 subunits were co-transfected, compared to cells transfected with only the α subunit.

FIGS. 3A-C show electrophysiology data for a produced cell line stably expressing all three NaV 1.7 subunits, indicating the signature response for NaV 1.7. FIG. 3A shows sodium currents in response to 20 ms depolarization pulses from −80 mV to +50 mV. FIG. 3B shows the resulting current-voltage (I-V) relationship for peak sodium channel currents. FIG. 3C shows the inactivation graph for the sodium channel.

FIG. 4 shows that cells stably expressing all three NaV 1.7 subunits responded to two known NaV activators, veratridine and scorpion venom, while control cells did not. The response was measured by a functional membrane potential cell-based assay.

FIGS. 5A and 5B show the activation of cells stably expressing NaV 1.7 in response to test compounds. FIG. 5A depicts the activation response of clone C44 (cells expressing all three NaV 1.7 subunits) when exposed to test compounds C18 and K21. FIG. 5B depicts the completely blocked response to the same test compounds of clone 60 (cells expressing only a NaV 1.7 alpha subunit). % Control was calculated relative to the response of the two clones to buffer only (i.e., no test compounds added)

### DETAILED DISCLOSURE

Unless otherwise defined, all technical and scientific terms used herein have the same meaning as commonly understood by one of ordinary skill in the art to that this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. Although a number of documents are cited herein, this citation does not constitute an admission that any of these documents forms part of the common general knowledge in the art. Throughout this specification and claims, the word “comprise,” or variations such as “comprises” or “comprising” will be understood to imply the inclusion of a stated integer or group of integers but not the exclusion of any other integer or group of integers. The materials, methods, and examples are illustrative only and not intended to be limiting.

In order that the present invention may be more readily understood, certain terms are first defined. Additional definitions are set forth throughout the specification.

As used herein, the term “native” protein (e.g., ion channel protein) refers to a protein that does not have a heterologous amino acid sequence appended or inserted to it. For example, “native NaV” used herein includes NaV proteins that do not have a tag sequence that is expressed on the polypeptide level. In some embodiments, a native NaV comprises all the subunits of a naturally occurring NaV where the subunits are intact and properly assembled.

The term “stable” or “stably expressing” is meant to distinguish the cells and cell lines of the invention from cells with transient expression as the terms “stable expression” and “transient expression” would be understood by a person of skill in the art.

The term “cell line” or “clonal cell line” refers to a population of cells that are all progeny of a single original cell. As used herein, cell lines are maintained in vitro in cell culture and may be frozen in aliquots to establish banks of clonal cells.

The term “stringent conditions” or “stringent hybridization conditions” describe temperature and salt conditions for hybridizing one or more nucleic acid probes to a nucleic acid sample and washing off probes that have not bound specifically to target nucleic acids in the sample. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. An example of stringent hybridization conditions is hybridization in 6×SSC at about 45° C., followed by at least one wash in 0.2×SSC, 0.1% SDS at 60° C. A further example of stringent hybridization conditions is hybridization in 6×SSC at about 45° C., followed by at least one wash in 0.2×SSC, 0.1% SDS at 65° C. Stringent conditions include hybridization in 0.5M sodium phosphate, 7% SDS at 65° C., followed by at least one

The phrase “percent identical” or “percent identity” in connection with amino acid and/or nucleic acid sequences refers to the similarity between at least two different sequences. This percent identity can be determined by standard alignment algorithms, for example, the Basic Local Alignment Tool (BLAST) described by Altshul et al. ((1990) J. Mol. Biol., 215: 403-410); the algorithm of Needleman et al. ((1970) J. Mol. Biol., 48: 444-453); or the algorithm of Meyers et al. ((1988) Comput. Appl. Biosci., 4: 11-17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) that has been incorporated into the ALIGN program (version 2.0), using a PAM120 weight residue table, a gap length penalty of 12 and a gap penalty of 4. The percent identity is usually calculated by comparing sequences of similar length. Protein analysis software matches similar sequences using measures of similarity assigned to various substitutions, deletions and other modifications, including conservative amino acid substitutions. For instance, GCG contains programs such as “Gap” and “Bestfit” that can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson, Methods Enzymol. 183:63-98 (1990); Pearson, Methods Mol. Biol. 132:185-219 (2000)). The length of polypeptide sequences compared for homology will generally be at least about 16 amino acid residues, usually at least about 20 residues, more usually at least about 24 residues, typically at least about 28 residues, and preferably more than about 35 residues.

The phrase “substantially as set out,” “substantially identical” or “substantially homologous” means that the relevant amino acid or nucleotide sequence will be identical to or have insubstantial differences (through conserved amino acid substitutions) in comparison to the sequences that are set out. Insubstantial differences include minor amino acid changes, such as 1 or 2 substitutions in a 50 amino acid sequence of a specified region.

A NaV “modulator” refers to a compound that alters a biological activity of a NaV, e.g., ion conductance via a NaV. A NaV modulator may act upon all or upon a specific subset of NaVs or NaV subunits. Modulators include, but are not limited to, agonists (potentiators or activators) and antagonists (inhibitors or blockers). A NaV agonist refers to a compound that increases a biological activity of a NaV. A NaV antagonist refers to a compound that decreases a biological activity of a NaV.

A “functional NaV” refers to a NaV that has one or more of the biological activities of a naturally occurring or endogenously expressed NaV. Biological activities of NaV include, but are not limited to, voltage-dependent sodium conductance, and can be assessed via pharmacological responses such as inhibition by lidocaine and tetrodotoxin (TTX). Other compounds that are pharmacologically active on NaV and can thus be used to assess the functionality of an introduced NaV include sodium channel openers—compounds that hold the channel in its open state, for example, veratridine, and various scorpion and other venoms.

A “heterologous” or “introduced” NaV subunit means that the NaV subunit is encoded by a polynucleotide introduced into a host cell, or by an endogenous NaV-coding sequence whose expression is activated (e.g., by gene activation technology) by externally introduced factors such as transcriptional regulatory elements. A “heterologous NaV” refers to NaV comprising one or more heterologous NaV subunits.

In a first aspect, the invention provides cells (e.g., isolated cells, clonal cells, or mixtures of clonal cells) and cell lines that express (e.g., stably) one or more heterologous (introduced) NaV subunits (e.g., native NaV subunits). The cells and cell lines may constitutively express the NaV subunits. The cells and cell lines may be modulated by channel openers such as veratridine and scorpion venom, or membrane voltage changes. The cells or cell lines may express one, two, three, or more heterologous NaV subunits (an alpha subunit and two types of beta subunits). In related embodiments, the cells or cell lines stably express a functional heterologous NaV. The NaV cells and cell lines of the invention have enhanced properties compared to cells and cell lines made by conventional methods. For example, the NaV cells and cell lines have enhanced stability of expression (even when maintained in culture without selective pressure such as antibiotics) and possess high Z′ values in cell-based assays. The cells and cell lines of the invention provide detectable signal-to-noise ratios, e.g., a signal-to-noise ratio greater than 1:1. The cells and cell lines of the invention provide reliable readouts when used in high throughput assays such as membrane potential assays, producing results that can match those from assays that are considered gold-standard in the field but too labor-intensive to carry out in a high-throughput manner (e.g., electrophysiology assays).

In various embodiments, the cells or cell lines of the invention express NaV at a consistent level of expression for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200 days or over 200 days, where consistent expression refers to a level of expression that does not vary by more than: 1%, 2%, 3%, 4%, 5%, 6%, 7%, 8% 9% or 10% over 2 to 4 days of continuous cell culture; 2%, 4%, 6%, 8%, 10% or 12% over 5 to 15 days of continuous cell culture; 2%, 4%, 6%, 8%, 10%, 12%, 14%, 16%, 18% or 20% over 16 to 20 days of continuous cell culture; 2%, 4%, 6%, 8%, 10%, 12%, 14%, 16%, 18%, 20%, 22%, 24% over 21 to 30 days of continuous cell culture; 2%, 4%, 6%, 8%, 10%, 12%, 14%, 16%, 18%, 20%, 22%, 24%, 26%, 28% or 30% over 30 to 40 days of continuous cell culture; 2%, 4%, 6%, 8%, 10%, 12%, 14%, 16%, 18%, 20%, 22%, 24%, 26%, 28% or 30% over 41 to 45 days of continuous cell culture; 2%, 4%, 6%, 8%, 10%, 12%, 14%, 16%, 18%, 20%, 22%, 24%, 26%, 28% or 30% over 45 to 50 days of continuous cell culture; 2%, 4%, 6%, 8%, 10%, 12%, 14%, 16%, 18%, 20%, 22%, 24%, 26%, 28%, 30% or 35% over 45 to 50 days of continuous cell culture, 2%, 4%, 6%, 8%, 10%, 12%, 14%, 16%, 18%, 20%, 22%, 24%, 26%, 28% or 30% over 50 to 55 days of continuous cell culture; 2%, 4%, 6%, 8%, 10%, 12%, 14%, 16%, 18%, 20%, 22%, 24%, 26%, 28%, 30% or 35% over 50 to 55 days of continuous cell culture; 1%, 2%, 3%, 4%, 5%, 10%, 15%, 20%, 25%, 30%, 35% or 40% over 55 to 75 days of continuous cell culture; 1%, 2%, 3%, 4%, 5%, 6%, 10%, 15%, 20%, 25%, 30%, 35%, 40% or 45% over 75 to 100 days of continuous cell culture; 1%, 2%, 3%, 4%, 5%, 6%, 10%, 15%, 20%, 25%, 30%, 35%, 40% or 45% over 101 to 125 days of continuous cell culture; 1%, 2%, 3%, 4%, 5%, 6%, 10%, 15%, 20%, 25%, 30%, 35%, 40% or 45% over 126 to 150 days of continuous cell culture; 1%, 2%, 3%, 4%, 5%, 6%, 10%, 15%, 20%, 25%, 30%, 35%, 40% or 45% over 151 to 175 days of continuous cell culture; 1%, 2%, 3%, 4%, 5%, 6%, 10%, 15%, 20%, 25%, 30%, 35%, 40% or 45% over 176 to 200 days of continuous cell culture; or 1%, 2%, 3%, 4%, 5%, 6%, 10%, 15%, 20%, 25%, 30%, 35%, 40% or 45% over more than 200 days of continuous cell culture.

The NaV can be from any mammal, including rat, mouse, rabbit, goat, dog, cow, pig or primate. The alpha subunit and each beta subunit can be from the same or different species. In a preferred embodiment, the NaV is human NaV, including human NaV 1.1, NaV 1.2, NaV 1.3, NaV 1.4, NaV 1.5, NaV 1.6, NaV 1.7, NaV 1.8, and NaV 1.9.

In various embodiments, the NaV alpha subunit may be any NaV alpha subunit, including any of the human NaV alpha subunits. Accordingly, in some embodiments, the cells of the invention may comprise a nucleic acid that encodes a NaV alpha 1 (SCN1A) (SEQ ID NO: 20); a NaV alpha 2 (SCN2A) (SEQ ID NO: 21); a NaV alpha 3 (SCN3A) (SEQ ID NO: 22); a NaV alpha 4 (SCN4A) (SEQ ID NO: 23); a NaV alpha 5 (SCN5A) (SEQ ID NO: 24); a NaV alpha 7 (SCN7A) (SEQ ID NO: 25) (alpha 6 and alpha 7 subunits are synonymous); a NaV alpha 8 (SCN8A) (SEQ ID NO: 26); a NaV alpha 9 (SCN9A) (SEQ ID NO: 27); a NaV alpha 10 (SCN10A) (SEQ ID NO: 28); or a NaV alpha 11 (SCN11A) (SEQ ID NO: 29). In some embodiments the NaV alpha subunit coding nucleic acid is selected from the group consisting of SEQ ID NOS: 6-15.

Any one of the NaV alpha subunits may be co-introduced, or sequentially introduced, and co-expressed with any one or more NaV beta subunits to generate the cells of the invention. In some embodiments, the cells stably expresses human NaV beta subunits, for example, a human NaV beta 1 subunit (SCN1B) (SEQ ID NO: 30), a human NaV beta 2 subunit (SCN2B) (SEQ ID NO: 31), a human NaV beta 3 subunit (SCN3B) (SEQ ID NO: 32) and a human NaV beta 4 subunit (SCN4B) (SEQ ID NO: 33). In some embodiments, the NaV beta subunit is encoded by a nucleic acid selected from the group consisting of SEQ ID NOS: 16-19. In some embodiments, the cells are triply transfected with nucleic acids encoding, and expresses, a human NaV alpha 9/SCN9A subunit, a human NaV beta1/SCN1B subunit and a human NaV beta 2/SCN2B subunit. In some embodiments, coding sequences for two or more of the introduced NaV subunits are placed on the same vector. In other embodiments, each subunit's coding sequence is placed on a different vector.

In some embodiments, the present cells and cell lines express an introduced alpha subunit, selected from any one of alpha 1-11, and an introduced beta subunit, selected from any one of beta 1-4, with each combination indicated by a “+” in the following table:

| | Beta 1 | Beta 2 | Beta 3 | Beta 4 |
|---|---|---|---|---|
| Alpha 1 | + | + | + | + |
| Alpha 2 | + | + | + | + |
| Alpha 3 | + | + | + | + |
| Alpha 4 | + | + | + | + |
| Alpha 5 | + | + | + | + |
| Alpha 7 | + | + | + | + |
| Alpha 8 | + | + | + | + |
| Alpha 9 | + | + | + | + |
| Alpha 10 | + | + | + | + |
| Alpha 11 | + | + | + | + |

These cells and cells lines can further express one or more introduced beta subunits independently selected from any one of beta 1-4. In some embodiments, the cells and cell lines of the invention express a NaV channel containing a combination of alpha and beta subunits as shown in the above table, and in further embodiments, the NaV channel in these cell lines further comprise one or more beta subunits selected from any one of beta 1-4.

The nucleic acid encoding the NaV subunit can be genomic DNA or cDNA. In some embodiments, the nucleic acid encoding the NaV subunit comprises one or more substitutions, insertions, or deletions that may or may not result in an amino acid substitution. NaV subunits with modifications within the scope of the invention retain at least one biological property, e.g., its ability to function as, or modulate, a voltage-gated sodium channel or to respond to ion channel openers such as veratridine and scorpion and other venoms and channel blockers such as lidocaine and tetrodotoxin (TTX). Accordingly, nucleic acid sequences substantially identical (e.g., at least about 85% sequence identity) or homologous (e.g., at least about 85% sequence homology) to the sequences disclosed herein are also encompassed by this invention. In some embodiment, the sequence identity can be about 85%, 90%, 95%, 96%, 97%, 98%, 99%, or higher. Alternatively, substantial identity or homology exists when the nucleic acid segments will hybridize under stringent hybridization conditions (e.g., highly stringent hybridization conditions) to the complement of the reference sequence.

In some embodiments, where the nucleotide mutation involves an amino acid substitution, the native amino acid may be replaced by a conservative or non-conservative substitution. In some embodiments, the sequence identity between the original and modified polypeptide sequences can be at least 85%, 90%, 95%, 96%, 97%, 98%, 99% or higher. Those of skill in the art will understand that a conservative amino acid substitution is one in which the amino acid side chains are similar in structure and/or chemical properties and the substitution should not substantially change the structural characteristics of the wild type sequence. In embodiments using a nucleic acid comprising a mutation, the mutation may be a random mutation or a site-specific mutation.

Conservative modifications will produce NaV receptors having functional and chemical characteristics similar to those of the unmodified NaV receptor. A “conservative amino acid substitution” is one in which an amino acid residue is substituted by another amino acid residue having a side chain R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See e.g. Pearson, Methods Mol. Biol. 243:307-31 (1994).

Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al., Science 256:1443-45 (1992). A “moderately conservative” replacement is any change having a nonnegative value in the PAM250 log-likelihood matrix.

In some embodiments, the NaV subunit-coding nucleic acid sequence further comprises an epitope tag. Such tags may encode, for example, yellow fluorescent protein (YFP), green fluorescent protein (GFP), 6x-HIS (SEQ ID NO: 35), myc, FLAG, or hemagglutinin (HA), S-tag, thioredoxin, autofluorescent proteins, GST, V5, TAP, CBP, BCCP, Maltose binding protein-tag, Nus-tag, Softag 1, Softag 3, Strep-tag, or a variant of the aforementioned. A tag may be used as a marker to determine the expression levels, intracellular localization, protein-protein interaction, regulation, and function of a NaV or a subunit thereof. A tag also may be used to facilitate protein purification and fractionation. These and other tag sequences are known to one of skill in the art and typically correspond to amino acid sequences that may be incorporated into expressed protein products and often selected based on the availability of robust antibodies or protein detection reagents that may be used to report their presence. However, tag sequences described herein are not meant to refer solely to sequences that may be used to modify, at the amino acid level, protein products encoded by the RNAs that are tagged, or to aid in the subsequent detection of any such modified protein products through use of the corresponding antibody or protein detection reagents. See, for example, discussions below in regard to using RNA tags used as “molecular beacons.”

Host cells used to produce a cell line of the invention may express one or more endogenous NaV proteins or lack expression of one or more of any NaV protein. The host cell may be a primary, germ, or stem cell, including an embryonic stem cell. The host cell may also be an immortalized cell. The host cell may be derived from a primary or immortalized cell from mesoderm, ectoderm, or endoderm layers. The host cell may be endothelial, epidermal, mesenchymal, neural, renal, hepatic, hematopoietic, or immune cells. For example, the host cells may be intestinal crypt or villi cells, clara cells, colon cells, intestinal cells, goblet cells, enterochromafin cells, enteroendocrine cells. The host cells may be eukaryotic, prokaryotic, mammalian, human, primate, bovine, porcine, feline, rodent, marsupial, murine or other cells. The host cells may also be nonmammalian, such as yeast, insect, fungus, plant, lower eukaryotes and prokaryotes. Such host cells may provide backgrounds that are more divergent for testing with a greater likelihood for the absence of expression products provided by the cell that may interact with the target. In preferred embodiments, the host cell is a mammalian cell. Examples of host cells that may be used for a cell line of the invention include but are not limited to: Chinese hamster ovary (CHO) cells, established neuronal cell lines, pheochromocytomas, neuroblastomas fibroblasts, rhabdomyosarcomas, dorsal root ganglion cells, CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26), MRC-5 (ATCC CCL 171), L-cells, HEK-293 (ATCC CRL1573), PC12 (ATCC CRL-1721), HEK293T (ATCC CRL-11268), RBL (ATCC CRL-1378), SH-SY5Y (ATCC CRL-2266), MDCK (ATCC CCL-34), SJ-RH30 (ATCC CRL-2061), HepG2 (ATCC HB-8065), ND7/23 (ECACC 92090903), CHO (ECACC 85050302), Vero (ATCC CCL 81), Caco-2 (ATCC HTB 37), K562 (ATCC CCL 243), Jurkat (ATCC TIB-152), Per.C6 (Crucell, Leiden, The Netherlands), Huvec (ATCC Human Primary PCS 100-010, Mouse CRL 2514, CRL 2515, CRL 2516), HuH-7D12 (ECACC 01042712), 293 (ATCC CRL 10852), A549 (ATCC CCL 185), 1MR-90 (ATCC CCL 186), MCF-7 (ATC HTB-22), U-2 OS (ATCC HTB-96), T84 (ATCC CCL 248), or any established cell line (polarized or nonpolarized) or any cell line available from repositories such as The American Type Culture Collection (ATCC, 10801 University Blvd. Manassas, Va. 20110-2209 USA) or European Collection of Cell Cultures (ECACC, Salisbury Wiltshire SP4 0JG England). One of ordinary skill in the art will understand that different known or unknown accessory factors that may interact with or alter the function or expression of the target depending on the choice of host cell type.

In one embodiment, the host cell is an embryonic stem cell that is then used as the basis for the generation of transgenic animals. Embryonic stem cells stably expressing at least one NaV subunit, and preferably a functional heterologous NaV receptor, may be implanted into organisms directly, or their nuclei may be transferred into other recipient cells and these may then be implanted in vivo for studying growth and development. The embryonic stem cells also may be used to create transgenic animals.

As will be appreciated by those of skill in the art, any vector that is suitable for use with the host cell may be used to introduce a nucleic acid encoding a NaV alpha or beta subunit into the host cell. The vectors comprising the alpha and each of the beta subunits may be the same type or may be of different types. Examples of vectors that may be used to introduce the NaV subunit-encoding nucleic acids into host cells include but are not limited to plasmids, viruses, including retroviruses and lentiviruses, cosmids, artificial chromosomes and may include for example, pFN11A (BIND) Flexi®, pGL4.31, pFC14A (HaloTag® 7) CMV pFC14K (HaloTag® 7) CMV Flexi®, pFN24A (HaloTag® 7) CMVd3 Flexi®, pFN24K (HaloTag® 7) CMVd3 Flexi®, HaloTag™ pHT2, pACT, pAdVAntage™, pALTER®-MAX, pBIND, pCAT®3-Basic, pCAT®3-Control, pCAT®3-Enhancer, pCAT®3-Promoter, pCI, pCMVTNT™, pG5luc, pSI, pTARGET™, pTNT™, pF12A RM Flexi®, pF12K RM Flexi®, pReg neo, pYES2/GS, pAd/CMV/V5-DEST Gateway® Vector, pAd/PL-DEST™ Gateway® Vector, Gateway® pDEST™27 Vector, Gateway® pEF-DEST51 Vector, Gateway® pcDNA™-DEST47 vector, pCMV/Bsd Vector, pEF6/His A, B, & C, pcDNA™6.2-DEST, pLenti6/TR, pLP-AcGFP1-C, pLPS-AcGFP1-N, pLP-IRESneo, pLP-TRE2, pLP-RevTRE, pLP-LNCX, pLP-CMV-HA, pLP-CMV-Myc, pLP-RetroQ, pLP-CMVneo, pCMV-Script, pcDNA3.1 Hygro, pcDNA3.1neo, pcDNA3.1puro, pSV2neo, pIRES puro, and pSV2 zeo. In some embodiments, the vectors comprise expression control sequences such as constitutive or conditional promoters. One of ordinary skill in the art will be able to select such sequences. For example, suitable promoters include but are not limited to CMV, TK, SV40 and EF-1α. In some embodiments, the promoters are inducible, temperature regulated, tissue specific, repressible, heat-shock, developmental, cell lineage specific, prokaryotic and/or eukaryotic expressible or temporal promoters or a combination or recombination of unmodified or mutagenized, randomized, shuffled sequences of any one or more of the above. Nucleic acids encoding NaV subunits are preferably constitutively expressed.

In some embodiments, the vector lacks a selectable marker or drug resistance gene. In other embodiments, the vector optionally comprises a nucleic acid encoding a selectable marker such as a protein that confers drug or antibiotic resistance. Each vector for a sequence encoding a different NaV subunit may have the same or a different drug resistance or other selectable marker. If more than one of the drug resistance markers are the same, simultaneous selection may be achieved by increasing the level of the drug. Suitable markers will be well-known to those of skill in the art and include but are not limited to genes conferring resistance to any one of the following: Neomycin/G418, Puromycin, hygromycin, Zeocin, methotrexate and blasticidin. Although drug selection (or selection using any other suitable selection marker) is not a required step, it may be used, if desired, to enrich the transfected cell population for stably transfected cells, provided that the transfected constructs are designed to confer drug resistance. If selection is accomplished using signaling probes, selection performed too soon following transfection can result in some positive cells that may only be transiently and not stably transfected. However, this can be minimized by allowing sufficient cell passage, allowing for dilution of transiently transfected cells, stably integrated cells that do not express the introduced DNA, or cells that generate RNA that may not be efficiently detected by the signaling probes.

In another aspect of the invention, cells and cell lines of the invention stably express NaV or a NaV subunit. To identify stable expression, a cell line's expression of each NaV subunit is measured over a time course and the expression levels are compared. Stable cell lines will continue expressing the NaV subunits throughout the time course at substantially the same level (e.g., no more than 40%, 30%, 20%, 15%, 10%, 5%, or 2% variation). In some aspects of the invention, the time course may be for at least one week, two weeks, three weeks, or four weeks; or at least one, two, three, four, five, six, seven, eight, or nine months, or at least any length of time in between. Isolated cells can be further characterized, such as by qRT-PCR and single end-point RT-PCR to determine the absolute and/or relative amounts of each NaV subunit being expressed, or by any other conventional method of protein expression analysis.

Cells and cell lines of the invention have the further advantageous property of providing assays with high reproducibility as evidenced by their Z′ factor. See Zhang J H, Chung T D, Oldenburg K R, “A Simple Statistical Parameter for Use in Evaluation and Validation of High Throughput Screening Assays.” J. Biomol. Screen. 1999; 4(2):67-73. Z′ values pertain to the quality of a cell or cell line because it reflects the degree to which a cell or cell line will respond consistently to modulators. Z′ is a statistical calculation that takes into account the signal-to-noise range and signal variability (i.e., from well to well) of the functional response to a reference compound across a multiwell plate. Z′ is calculated using data obtained from multiple wells with a positive control and multiple wells with a negative control. The ratio of their combined standard deviations multiplied by three to the difference in their mean values is subtracted from one to give the Z′ factor, according the equation below:

Z′factor=1−((3σ_{positive control}+3σ_{negative control})/(μ_{positive control}−μ_{negative control}))

The theoretical maximum Z′ factor is 1.0, which would indicate an ideal assay with no variability and limitless dynamic range. As used herein, a “high Z′” refers to a Z′ factor of Z′ of at least 0.6, at least 0.7, at least 0.75 or at least 0.8, or any decimal in between 0.6 and 1.0. A score less than 0 is undesirable because it indicates that there is overlap between positive and negative controls. In the industry, for simple cell-based assays, Z′ scores up to 0.3 are considered marginal scores, Z′ scores between 0.3 and 0.5 are considered acceptable, and Z′ scores above 0.5 are considered excellent. Cell-free or biochemical assays may approach higher Z′ scores, but Z′ scores for cell-based systems tend to be lower because cell-based systems are complex.

As those of ordinary skill in the art will recognize, historically, cell-based assays using cells expressing a single chain protein do not typically achieve a Z′ higher than 0.5 to 0.6. Such cells would not be reliable to use in an assay because the results are not reproducible. Cells and cell lines of the invention, on the other hand, have high Z′ values and advantageously produce consistent results in assays. NaV cells and cell lines of the invention provide the basis for high throughput screening (HTS) compatible assays because they generally have Z′ factors at least 0.7. In some aspects of the invention, the cells and cell lines result in Z′ of at least 0.3, at least 0.4, at least 0.5, at least 0.6, at least 0.7, or at least 0.8. In other aspects of the invention, the cells and cell lines of the invention result in a Z′ of at least 0.7, at least 0.75 or at least 0.8 maintained for multiple passages, e.g., between 5-20 passages, including any integer in between 5 and 20. In some aspects of the invention, the cells and cell lines result in a Z′ of at least 0.7, at least 0.75 or at least 0.8 maintained for 1, 2, 3, 4 or 5 weeks or 2, 3, 4, 5, 6, 7, 8 or 9 months, including any period of time in between.

Also according to the invention, cells and cell lines that express a recombinant form of a naturally occurring NaV hetero-multimer (in contrast to cell lines expressing non-naturally occurring combinations of alpha and beta subunits or expressing just the alpha or beta subunits) can be characterized for NaV functions, e.g., sodium ion conductance. In some embodiments, the cells and cell lines of the invention display “physiologically relevant” activity of an introduced ion channel. As used herein, “physiological relevance” refers to a property of a cell or a cell line expressing an introduced ion channel whereby the introduced ion channel behaves, e.g., responds to a modulator, in substantially the same way as a naturally occurring channel of the same type, e.g., a naturally occurring receptor having the same combination of alpha and beta subunits. Cells and cell lines of this invention preferably demonstrate comparable function to cells that normally express endogenous (native) NaV (e.g., primary cells) in a suitable assay, such as a membrane potential assay using sodium as a NaV activator, an electrophysiology assay, and a binding or panning assay. Such comparisons are used to determine a cell or cell line's physiological relevance.

In another aspect of the invention, modulators identified using the cell lines of the invention can be used in additional assays to confirm functionality. A further advantageous property of cells and cell lines of the inventions is that modulators identified in initial screening are functional in secondary functional assays, e.g., membrane potential assay or an electrophysiology assay. As those of ordinary skill in the art will recognize, compounds identified in initial screening assays typically must be modified, such as by combinatorial chemistry, medicinal chemistry or synthetic chemistry, for their derivatives or analogs to be functional in secondary functional assays. However, due to the high physiological relevance of the present cells and cell lines, compounds identified therewith may not require such “coarse” tuning.

In some embodiments, the cells and cell lines of the invention have increased sensitivity to modulators of NaV. Cells and cell lines of the invention respond to modulators with physiological range EC₅₀ or IC₅₀ values for NaV. As used herein, EC₅₀ refers to the concentration of a compound or substance required to induce a half-maximal activating response in the cell or cell line. As used herein, IC₅₀ refers to the concentration of a compound or substance required to induce a half-maximal inhibitory response in the cell or cell line. EC₅₀ and IC₅₀ values may be determined using techniques that are well-known in the art, for example, a dose-response curve that correlates the concentration of a compound or substance to the response of the NaV-expressing cell line. For example, the IC₅₀ for tetrodotoxin (TTX) in a cell line of the invention is about 1-100 nM, e.g., 1, 2, 5, 10, 20, 30, 40, 50, 60, 70, 80, or 90 nM.

In some embodiments, properties of the cells and cell lines of the invention, such as stability, physiological relevance, reproducibility in an assay (Z′), or physiological EC₅₀ or IC₅₀ values, are achievable under specific culture conditions. In some embodiments, the culture conditions are standardized and rigorously maintained without variation, for example, by automation. Culture conditions may include any suitable conditions under which the cells or cell lines are grown and may include those known in the art. A variety of culture conditions may result in advantageous biological properties for NAV, or its mutants or allelic variants.

In other embodiments, the cells and cell lines of the invention with desired properties, such as stability, physiological relevance, reproducibility in an assay (Z′), or physiological EC₅₀ or IC₅₀ values, can be obtained within one month or less. For example, the cells or cell lines may be obtained within 2, 3, 4, 5, or 6 days, or within 1, 2, 3 or 4 weeks, or any length of time in between.

The present cells and cell lines can be used in a collection or panel, each set of cells or each cell line expressing one form of NaV [e.g., NaV comprised of various combinations (e.g., dimers, trimers, etc.) of alpha and beta subunits or variants (e.g., mutants, fragments, or spliced variants) of the subunits, or a mono- or multi-mer of only an alpha or beta subunit]. The collection may include, for example, cell lines expressing two or more of the aforementioned NaV receptors. In some embodiments, the collection or panel may further comprise members expressing the same NaV or expressing control proteins.

When collections or panels of cells or cell lines are produced, e.g., for drug screening, the cells or cell lines in the collection or panel may be matched such that they are the same (including substantially the same) with regard to one or more selective physiological properties. The “same physiological property” in this context means that the selected physiological property is similar enough amongst the members in the collection or panel such that the cell collection or panel can produce reliable results in drug screening assays; for example, variations in readouts in a drug screening assay will be due to, e.g., the different biological activities of test compounds on cells expressing different forms of NaV, rather than due to inherent variations in the cells. For example, the cells or cell lines may be matched to have the same growth rate, i.e., growth rates with no more than one, two, three, four, or five hour difference amongst the members of the cell collection or panel. This may be achieved by, for example, binning cells by their growth rate into five, six, seven, eight, nine, or ten groups, and creating a panel using cells from the same binned group. Methods of determining cell growth rate are well known in the art. The cells or cell lines in a panel also can be matched to have the same Z′ factor (e.g., Z′ factors that do not differ by more than 0.1), NaV expression level (e.g., NaV expression levels that do not differ by more than 5%, 10%, 15%, 20%, 25%, or 30%), adherence to tissue culture surfaces, and the like. Matched cells and cell lines can be grown under identical conditions, achieved by, e.g., automated parallel processing, to maintain the selected physiological property.

Matched cell panels of the invention can be used to, for example, identify modulators with defined activity (e.g., agonist or antagonist) on NaV; to profile compound activity across different forms of NaV; to identify modulators active on just one form of NaV; and to identify modulators active on just a subset of NaVs. The matched cell panels of the invention allow high throughput screening. Screenings that used to take months to accomplish can now be accomplished within weeks.

To make cells and cell lines of the invention, one can use, for example, the technology described in U.S. Pat. No. 6,692,965 and WO/2005/079462. Both of these documents are incorporated herein by reference in their entirety. This technology provides real-time assessment of millions of cells such that any desired number of clones (from hundreds to thousands of clones). Using cell sorting techniques, such as flow cytometric cell sorting (e.g., with a FACS machine) or magnetic cell sorting (e.g., with a MACS machine), one cell per well is automatically deposited with high statistical confidence in a culture vessel (such as a 96 well culture plate). The speed and automation of the technology allows multigene recombinant cell lines to be readily isolated.

Using the technology, the RNA sequence for each NaV subunit may be detected using a signaling probe, also referred to as a molecular beacon or fluorogenic probe. In some embodiments, the vector containing the NaV subunit-coding sequence has an additional sequence coding for an RNA tag sequence. “Tag sequence” refers to a nucleic acid sequence that is an expressed RNA or portion of an RNA that is to be detected by a signaling probe. Signaling probes may detect a variety of RNA sequences, any of which may be used as tags, including those encoding peptide and protein tags described above. Signaling probes may be directed against the tag by designing the probes to include a portion that is complementary to the sequence of the tag. The tag sequence may be a 3′ untranslated region of the plasmid that is cotranscribed with a NaV transcript and comprises a target sequence for signaling probe binding. The tag sequence can be in frame with the protein-coding portion of the message of the gene or out of frame with it, depending on whether one wishes to tag the protein produced. Thus, the tag sequence does not have to be translated for detection by the signaling probe. The tag sequences may comprise multiple target sequences that are the same or different, wherein one signaling probe hybridizes to each target sequence. The tag sequence may be located within the RNA encoding the gene of interest, or the tag sequence may be located within a 5′- or 3′-untranslated region. The tag sequences may be an RNA having secondary structure. The structure may be a three-arm junction structure. In some embodiments, the signaling probe detects a sequence within the NaV subunit-coding sequence.

Nucleic acids comprising a sequence encoding a NaV subunit, optionally a sequence coding for a tag sequence, and optionally a nucleic acid encoding a selectable marker may be introduced into selected host cells by well known methods. The methods include but not limited to transfection, viral delivery, protein or peptide mediated insertion, coprecipitation methods, lipid based delivery reagents (lipofection), cytofection, lipopolyamine delivery, dendrimer delivery reagents, electroporation or mechanical delivery. Examples of transfection reagents are GENEPORTER, GENEPORTER2, LIPOFECTAMINE, LIPOFECTAMINE 2000, OLIGOFECTAMINE, TRANSFAST, TRANSFECTAM, GENESHUTTLE, TROJENE, GENESILENCER, X-TREMEGENE, PERFECTIN, CYTOFECTIN, SIPORT, UMFECTOR, FUGENE 6, FUGENE HD, TFX-10, TFX-20, TFX-50, SIFECTOR, TRANSIT-LT1, TRANSIT-LT2, TRANSIT-EXPRESS, IFECT, RNAI SHUTTLE, METAFECTENE, LYOVEC, LIPOTAXI, GENEERASER, GENEJUICE, CYTOPURE, JETSI, JETPEI, MEGAFECTIN, POLYFECT, TRANSMESSANGER, RNAiFECT, SUPERFECT, EFFECTENE, TF-PEI-KIT, CLONFECTIN, AND METAFECTINE.

Following transfection of the DNA constructs into cells and subsequent drug selection (if used), or following gene activation as described above, molecular beacons (e.g., fluorogenic probes), each of which is targeted to a different tag sequence and differentially labeled, may be introduced into the cells, and a flow cytometric cell sorter is used to isolate cells positive for their signals (multiple rounds of sorting may be carried out). In one embodiment, the flow cytometric cell sorter is a FACS machine. MACS (magnetic cell sorting) or laser ablation of negative cells using laser-enabled analysis and processing can also be used. Other fluorescence plate readers, including those that are compatible with high-throughput screening can also be used. Signal-positive cells have taken up and may have integrated into their genomes at least one copy of the introduced NaV sequence(s). Cells introduced with one or more of the NaV subunits are identified. By way of example, the NaV subunit sequences may be integrated at different locations of the genome in the cell. The expression level of the introduced genes encoding the NaV subunits may vary based upon copy number or integration site. Further, cells comprising one or more of the NaV subunits may be obtained wherein one or more of the introduced genes encoding a NaV subunit is episomal or results from gene activation.

Signaling probes useful in this invention are known in the art and generally are oligonucleotides comprising a sequence complementary to a target sequence and a signal emitting system so arranged that no signal is emitted when the probe is not bound to the target sequence and a signal is emitted when the probe binds to the target sequence. By way of non-limiting illustration, the signaling probe may comprise a fluorophore and a quencher positioned in the probe so that the quencher and fluorophore are brought together in the unbound probe. Upon binding between the probe and the target sequence, the quencher and fluorophore separate, resulting in emission of signal. International publication WO/2005/079462, for example, describes a number of signaling probes that may be used in the production of the present cells and cell lines. The methods described above for introducing nucleic acids into cells may be used to introduce signaling probes.

Where tag sequences are used, the vector for each of the NaV subunit can comprise the same or a different tag sequence. Whether the tag sequences are the same or different, the signaling probes may comprise different signal emitters, such as different colored fluorophores and the like so that expression of each subunit may be separately detected. By way of illustration, the signaling probe that specifically detects NaV alpha subunit mRNA can comprise a red fluorophore, the probe that detects the first NaV beta subunit can comprise a green fluorophore, and the probe that detects the second NaV beta subunit can comprise a blue fluorophore. Those of skill in the art will be aware of other means for differentially detecting the expression of the three subunits with a signaling probe in a triply transfected cell.

In one embodiment, the signaling probes are designed to be complementary to either a portion of the RNA encoding a NaV subunit or to portions of their 5′ or 3′ untranslated regions. Even if the signaling probe designed to recognize a messenger RNA of interest is able to detect spuriously endogenously expressed target sequences, the proportion of these in comparison to the proportion of the sequence of interest produced by transfected cells is such that the sorter is able to discriminate the two cell types.

The expression level of an introduced NaV subunit may vary from cell line to cell line. The expression level in a cell line also may decrease over time due to epigenetic events such as DNA methylation and gene silencing and loss of transgene copies. These variations can be attributed to a variety of factors, for example, the copy number of the transgene taken up by the cell, the site of genomic integration of the transgene, and the integrity of the transgene following genomic integration. One may use FACS to evaluate expression levels. Cells expressing an introduced NaV subunit at desired levels can be isolated by, e.g., FACS. Signaling probes also may be re-applied to previously generated cells or cell lines, for example, to determine if and to what extent the cells are still positive for any one or more of the RNAs for which they were originally isolated.

Once cells expressing all three NaV subunits are isolated, they may be cultured for a length of time sufficient to identify those stably expressing all the desired subunits. In another embodiment of the invention, adherent cells can be adapted to suspension before or after cell sorting and isolating single cells. In other embodiments, isolated cells may be grown individually or pooled to give rise to populations of cells. Individual or multiple cell lines may also be grown separately or pooled. If a pool of cell lines is producing a desired activity or has a desired property, it can be further fractionated until the cell line or set of cell lines having this effect is identified. Pooling cells or cell lines may make it easier to maintain large numbers of cell lines without the requirements for maintaining each separately. Thus, a pool of cells or cell lines may be enriched for positive cells. An enriched pool may have at least 50%, at least 60%, at least 70%, at least 80%, at least 90% or 100% are positive for the desired property or activity.

In a further aspect, the invention provides a method for producing the cells and cell lines of the invention. In one embodiment, the method comprises the steps of: a) providing a plurality of cells that express mRNA(s) encoding a NaV; b) dispersing cells individually into individual culture vessels, thereby providing a plurality of separate cell cultures; c) culturing the cells under a set of desired culture conditions using automated cell culture methods characterized in that the conditions are substantially identical for each of the separate cell cultures, during which culturing the number of cells in each separate cell culture is normalized, and wherein the separate cultures are passaged on the same schedule; d) assaying the separate cell cultures for at least one desired characteristic of the NaV protein (e.g., stable expression) at least twice; and e) identifying a separate cell culture that has the desired characteristic in both assays.

According to the method, the cells are cultured under a desired set of culture conditions. The conditions can be any desired conditions. Those of skill in the art will understand what parameters are comprised within a set of culture conditions. For example, culture conditions include but are not limited to: the media (Base media (DMEM, MEM, RPMT, serum-free, with serum, fully chemically defined, without animal-derived components), mono and divalent ion (sodium, potassium, calcium, magnesium) concentration, additional components added (amino acids, antibiotics, glutamine, glucose or other carbon source, HEPES, channel blockers, modulators of other targets, vitamins, trace elements, heavy metals, co-factors, growth factors, anti-apoptosis reagents), fresh or conditioned media, with HEPES, pH, depleted of certain nutrients or limiting (amino acid, carbon source)), level of confluency at which cells are allowed to attain before split/passage, feeder layers of cells, or gamma-irradiated cells, CO2, a three gas system (oxygen, nitrogen, carbon dioxide), humidity, temperature, still or on a shaker, and the like, which will be well known to those of skill in the art.

The cell culture conditions may be chosen for convenience or for a particular desired use of the cells. Advantageously, the invention provides cells and cell lines that are optimally suited for a particular desired use. That is, in embodiments of the invention in which cells are cultured under conditions for a particular desired use, cells are selected that have desired characteristics under the condition for the desired use. By way of illustration, if cells will be used in assays in plates where it is desired that the cells are adherent, cells that display adherence under the conditions of the assay may be selected. Similarly, if the cells will be used for protein production, cells may be cultured under conditions appropriate for protein production and selected for advantageous properties for this use.

In some embodiments, the method comprises the additional step of measuring the growth rates of the separate cell cultures. Growth rates may be determined using any of a variety of techniques means that will be well known to the skilled worker. Such techniques include but are not limited to measuring ATP, cell confluency, light scattering, optical density (e.g., OD 260 for DNA). Preferably growth rates are determined using means that minimize the amount of time that the cultures spend outside the selected culture conditions.

In some embodiments, cell confluency is measured and growth rates are calculated from the confluency values. In some embodiments, cells are dispersed and clumps removed prior to measuring cell confluency for improved accuracy. Means for monodispersing cells are well-known and can be achieved, for example, by addition of a dispersing reagent to a culture to be measured. Dispersing agents are well-known and readily available, and include but are not limited to enzymatic dispering agents, such as trypsin, and EDTA-based dispersing agents. Growth rates can be calculated from confluency date using commercially available software for that purpose such as HAMILTON VECTOR. Automated confluency measurement, such as using an automated microscopic plate reader is particularly useful. Plate readers that measure confluency are commercially available and include but are not limited to the CLONE SELECT IMAGER (Genetix). Typically, at least 2 measurements of cell confluency are made before calculating a growth rate. The number of confluency values used to determine growth rate can be any number that is convenient or suitable for the culture. For example, confluency can be measured multiple times over e.g., a week, 2 weeks, 3 weeks or any length of time and at any frequency desired.

When the growth rates are known, according to the method, the plurality of separate cell cultures are divided into groups by similarity of growth rates. By grouping cultures into growth rate bins, one can manipulate the cultures in the group together, thereby providing another level of standardization that reduces variation between cultures. For example, the cultures in a bin can be passaged at the same time, treated with a desired reagent at the same time, etc. Further, functional assay results are typically dependent on cell density in an assay well. A true comparison of individual clones is only accomplished by having them plated and assayed at the same density. Grouping into specific growth rate cohorts enables the plating of clones at a specific density that allows them to be functionally characterized in a high throughput format.

The range of growth rates in each group can be any convenient range. It is particularly advantageous to select a range of growth rates that permits the cells to be passaged at the same time and avoid frequent renormalization of cell numbers. Growth rate groups can include a very narrow range for a tight grouping, for example, average doubling times within an hour of each other. But according to the method, the range can be up to 2 hours, up to 3 hours, up to 4 hours, up to 5 hours or up to 10 hours of each other or even broader ranges. The need for renormalization arises when the growth rates in a bin are not the same so that the number of cells in some cultures increases faster than others. To maintain substantially identical conditions for all cultures in a bin, it is necessary to periodically remove cells to renormalize the numbers across the bin. The more disparate the growth rates, the more frequently renormalization is needed.

In step d) the cells and cell lines may be tested for and selected for any physiological property including but not limited to: a change in a cellular process encoded by the genome; a change in a cellular process regulated by the genome; a change in a pattern of chromosomal activity; a change in a pattern of chromosomal silencing; a change in a pattern of gene silencing; a change in a pattern or in the efficiency of gene activation; a change in a pattern or in the efficiency of gene expression; a change in a pattern or in the efficiency of RNA expression; a change in a pattern or in the efficiency of RNAi expression; a change in a pattern or in the efficiency of RNA processing; a change in a pattern or in the efficiency of RNA transport; a change in a pattern or in the efficiency of protein translation; a change in a pattern or in the efficiency of protein folding; a change in a pattern or in the efficiency of protein assembly; a change in a pattern or in the efficiency of protein modification; a change in a pattern or in the efficiency of protein transport; a change in a pattern or in the efficiency of transporting a membrane protein to a cell surface change in growth rate; a change in cell size; a change in cell shape; a change in cell morphology; a change in % RNA content; a change in % protein content; a change in % water content; a change in % lipid content; a change in ribosome content; a change in mitochondrial content; a change in ER mass; a change in plasma membrane surface area; a change in cell volume; a change in lipid composition of plasma membrane; a change in lipid composition of nuclear envelope; a change in protein composition of plasma membrane; a change in protein; composition of nuclear envelope; a change in number of secretory vesicles; a change in number of lysosomes; a change in number of vacuoles; a change in the capacity or potential of a cell for: protein production, protein secretion, protein folding, protein assembly, protein modification, enzymatic modification of protein, protein glycosylation, protein phosphorylation, protein dephosphorylation, metabolite biosynthesis, lipid biosynthesis, DNA synthesis, RNA synthesis, protein synthesis, nutrient absorption, cell growth, mitosis, meiosis, cell division, to dedifferentiate, to transform into a stem cell, to transform into a pluripotent cell, to transform into a omnipotent cell, to transform into a stem cell type of any organ (i.e. liver, lung, skin, muscle, pancreas, brain, testis, ovary, blood, immune system, nervous system, bone, cardiovascular system, central nervous system, gastro-intestinal tract, stomach, thyroid, tongue, gall bladder, kidney, nose, eye, nail, hair, taste bud), to transform into a differentiated any cell type (i.e. muscle, heart muscle, neuron, skin, pancreatic, blood, immune, red blood cell, white blood cell, killer T-cell, enteroendocrine cell, taste, secretory cell, kidney, epithelial cell, endothelial cell, also including any of the animal or human cell types already listed that can be used for introduction of nucleic acid sequences), to uptake DNA, to uptake small molecules, to uptake fluorogenic probes, to uptake RNA, to adhere to solid surface, to adapt to serum-free conditions, to adapt to serum-free suspension conditions, to adapt to scaled-up cell culture, for use for large scale cell culture, for use in drug discovery, for use in high throughput screening, for use in a functional cell based assay, for use in membrane potential assays, for use in reporter cell based assays, for use in ELISA studies, for use in in vitro assays, for use in vivo applications, for use in secondary testing, for use in compound testing, for use in a binding assay, for use in palming assay, for use in an antibody panning assay, for use in imaging assays, for use in microscopic imaging assays, for use in multiwell plates, for adaptation to automated cell culture, for adaptation to miniaturized automated cell culture, for adaptation to large-scale automated cell culture, for adaptation to cell culture in multiwell plates (6, 12, 24, 48, 96, 384, 1536 or higher density), for use in cell chips, for use on slides, for use on glass slides, for microarray on slides or glass slides, for use in biologics production, and for use in the production of reagents for research.

Tests that may be used to characterize cells and cell lines of the invention and/or matched panels of the invention include but are not limited to: amino acid analysis, DNA sequencing, protein sequencing, NMR, a test for protein transport, a test for nucelocytoplasmic transport, a test for subcellular localization of proteins, a test for subcellular localization of nucleic acids, microscopic analysis, submicroscopic analysis, fluorescence microscopy, electron microscopy, confocal microscopy, laser ablation technology, cell counting and Dialysis. The skilled worker would understand how to use any of the above-listed tests.

According to the method, cells may be cultured in any cell culture format so long as the cells or cell lines are dispersed in individual cultures prior to the step of measuring growth rates. For example, for convenience, cells may be initially pooled for culture under the desired conditions and then individual cells separated one cell per well or vessel. Cells may be cultured in multi-well tissue culture plates with any convenient number of wells. Such plates are readily commercially available and will be well knows to a person of skill in the art. In some cases, cells may preferably be cultured in vials or in any other convenient format, the various formats will be known to the skilled worker and are readily commercially available.

In embodiments comprising the step of measuring growth rate, prior to measuring growth rates, the cells are cultured for a sufficient length of time for them to acclimate to the culture conditions. As will be appreciated by the skilled worker, the length of time will vary depending on a number of factors such as the cell type, the chosen conditions, the culture format and may be any amount of time from one day to a few days, a week or more.

Preferably, each individual culture in the plurality of separate cell cultures is maintained under substantially identical conditions a discussed below, including a standardized maintenance schedule. Another advantageous feature of the method is that large numbers of individual cultures can be maintained simultaneously, so that a cell with a desired set of traits may be identified even if extremely rare. For those and other reasons, according to the invention, the plurality of separate cell cultures are cultured using automated cell culture methods so that the conditions are substantially identical for each well. Automated cell culture prevents the unavoidable variability inherent to manual cell culture.

Any automated cell culture system may be used in the method of the invention. A number of automated cell culture systems are commercially available and will be well-known to the skilled worker. In some embodiments, the automated system is a robotic system. Preferably, the system includes independently moving channels, a multichannel head (for instance a 96-tip head) and a gripper or cherry-picking arm and a HEPA filtration device to maintain sterility during the procedure. The number of channels in the pipettor should be suitable for the format of the culture. Convenient pipettors have, e.g., 96 or 384 channels. Such systems are known and are commercially available. For example, a MICROLAB STAR™ instrument (Hamilton) may be used in the method of the invention. The automated system should be able to perform a variety of desired cell culture tasks. Such tasks will be known by a person of skill in the art. They include but are not limited to: removing media, replacing media, adding reagents, cell washing, removing wash solution, adding a dispersing agent, removing cells from a culture vessel, adding cells to a culture vessel an the like.

The production of a cell or cell line of the invention may include any number of separate cell cultures. However, the advantages provided by the method increase as the number of cells increases. There is no theoretical upper limit to the number of cells or separate cell cultures that can be utilized in the method. According to the invention, the number of separate cell cultures can be two or more but more advantageously is at least 3, 4, 5, 6, 7, 8, 9, 10 or more separate cell cultures, for example, at least 12, at least 15, at least 20, at least 24, at least 25, at least 30, at least 35, at least 40, at least 45, at least 48, at least 50, at least 75, at least 96, at least 100, at least 200, at least 300, at least 384, at least 400, at least 500, at least 1000, at least 10,000, at least 100,000, at least 500,000 or more.

The ease to isolate and re-isolate from a mixed cell population those cells with desired properties (e.g., expressing desired RNAs at appropriate levels) makes it possible to maintain cell lines under no or minimal drug selection pressure. Selection pressure is applied in cell culture to select cells with desired sequences or traits, and is usually achieved by linking the expression of a polypeptide of interest with the expression of a selection marker that imparts to the cells resistance to a corresponding selective agent or pressure. Antibiotic selection includes, without limitation, the use of antibiotics (e.g., puromycin, neomycin, G418, hygromycin, bleomycin and the like). Non-antibiotic selection includes, without limitation, the use of nutrient deprivation, exposure to selective temperatures, exposure to mutagenic conditions and expression of fluorescent markers where the selection marker may be e.g., glutamine synthetase, dihydrofolate reductase (DHFR), oabain, thymidine kinase (TK), hypoxanthine guanine phosphororibosyltransferase (HGPRT) or a fluorescent protein such as GFP. In the instant aspects of the invention, none of such selection steps are applied to the cells in culture. In some preferred embodiments, cells and cell lines of the invention are maintained in culture without any selective pressure. In further embodiments, cells and cell lines are maintained without any antibiotics. As used herein, cell maintenance refers to culturing cells after they have been selected for their NaV expression through, e.g., cell sorting. Maintenance does not refer to the optional step of growing cells in a selective drug (e.g., an antibiotic) prior to cell sorting where drug resistance marker(s) introduced into the cells allow enrichment of stable transfectants in a mixed population.

Drug-free cell maintenance provides a number of advantages. For examples, drug-resistant cells do not always express the co-transfected transgene of interest at adequate levels, because the selection relies on survival of the cells that have taken up the drug resistant gene, with or without the transgene. Further, selective drugs are often mutagenic or otherwise interferes the physiology of the cells, leading to skewed results in cell-based assays. For example, selective drugs may decrease susceptibility to apoptosis (Robinson et al., Biochemistry, 36(37):11169-11178 (1997)), increase DNA repair and drug metabolism (Deffie et al., Cancer Res. 48(13):3595-3602 (1988)), increase cellular pH (Thiebaut et al., J Histochem Cytochem. 38(5):685-690 (1990); Roepe et al., Biochemistry. 32(41):11042-11056 (1993); Simon et al., Proc Natl Acad Sci USA. 91(3):1128-1132 (1994)), decrease lysosomal and endosomal pH (Schindler et al., Biochemistry. 35(9):2811-2817 (1996); Altan et al., J Exp Med. 187(10):1583-1598 (1998)), decrease plasma membrane potential (Roepe et al., Biochemistry. 32(41):11042-11056 (1993)), increase plasma membrane conductance to chloride (Gill et al., Cell. 71(1):23-32 (1992)) and ATP (Abraham et al., Proc Natl Acad Sci USA. 90(1):312-316 (1993)), and increase rates of vesicle transport (Altan et al., Proc Natl Acad Sci USA. 96(8):4432-4437 (1999)). GFP, a commonly used non-antibiotic selective marker, may cause cell death in certain cell lines (Hanazono et al., Hum Gene Ther. 8(11):1313-1319 (1997)). Thus, the cells and cell lines of this invention allow screening assays that are free from any artifact caused by selective drugs or markers. In some preferred embodiments, cells are not cultured with selective drugs such as antibiotics before or after cell sorting so that cells with desired properties are isolated by sorting even without beginning with an enriched cell population.

In another aspect, the invention provides methods of using the cells and cell lines of the invention. The cells and cell lines of the invention may be used in any application for which a functional NaV subunit(s) or complete NaV ion channel is needed. The cells and cell lines may be used, for example, in an in vitro cell-based assay or an in vivo assay where the cells are implanted in an animal (e.g., a non-human mammal) to, e.g., screen for NaV modulators; produce protein for crystallography and binding studies; and investigate compound selectivity and dosing, receptor/compound binding kinetic and stability, and effects of receptor expression on cellular physiology (e.g., electrophysiology, protein trafficking, protein folding, and protein regulation). The present cells and cell lines also can be used in knock down studies to study the roles of specific NaV subunits.

Cell lines expressing various combinations of alpha and beta subunits (e.g., naturally occurring heterotrimers or nonnaturally occurring heterotrimers) can be used separately or together as a collection to identify NaV modulators, including those specific for a particular NaV, a particular subunit of a NaV, or a particular combination of NaV subunits, and to obtain information about the activities of individual subunits. The invention also provides methods for using modulators specific for particular modified forms; such information may be useful in determining whether NaV has naturally occurring modified forms. Using the present cell and cell lines can help determine whether different forms of NaV are implicated in different NaV pathologies and allow selection of disease- or tissue-specific NaV modulators for highly targeted treatment of NaV-related pathologies.

As used herein, a “modulator” includes any substance or compound that has modulating activity with respect to at least one NaV subunit. The modulator can be a NaV agonist (potentiator or activator) or antagonist (inhibitor or blocker), including partial agonists or antagonists, selective agonists or antagonists and inverse agonists, and can be an allosteric modulator. A substance or compound is a modulator even if its modulating activity changes under different conditions or concentrations. In some aspects of the invention, the modulator alters the selectivity of an ion channel. For example, a modulator may affect what ions are able to pass through an ion channel.

To identify a NaV modulator, one can expose a cell line of the invention to a test compound under conditions in which the NaV would be expected to be functional and detect a statistically significant change (e.g., p<0.05) in NaV activity compared to a suitable control, e.g., cells from the cell line that are not contacted with the test compound. Alternatively, or in addition positive and/or negative controls using known agonists or antagonists, cells expressing different combinations of NaV subunits may be used. In some embodiments, the NaV activity to be detected and/or measured is membrane depolarization, change in membrane potential, or fluorescence resulting from such membrane changes.

In some embodiments, one or more cell lines of the invention are exposed to a plurality of test compounds, for example, a library of test compounds. A library of test compounds can be screened using the cell lines of the invention to identify one or more modulators. The test compounds can be chemical moieties including small molecules, polypeptides, peptides, peptide mimetics, antibodies or antigen-binding portions thereof. In the case of antibodies, they may be non-human antibodies, chimeric antibodies, humanized antibodies, or fully human antibodies. The antibodies may be intact antibodies comprising a full complement of heavy and light chains or antigen-binding portions, including antibody fragments (such as Fab and Fab, Fab′, F(ab′)₂, Fd, Fv, dAb and the like), single subunit antibodies (scFv), single domain antibodies, all or an antigen-binding portion of a heavy or light chain.

In some embodiments, prior to exposure to a test compound, the cells may be modified by pretreatment with, for example, enzymes, including mammalian or other animal enzymes, plant enzymes, bacterial enzymes, protein modifying enzymes and lipid modifying enzymes, and enzymes in the oral cavity, gastrointestinal tract, stomach or saliva. Such enzymes can include, for example, kinases, proteases, phosphatases, glycosidases, oxidoreductases, transferases, hydrolases, lyases, isomerases, ligases and the like. For example, in some embodiments, cells are pretreated with at least one proteolytic enzyme such as trypsin or furin. Alternatively, the cells may be exposed to the test compound first followed by treatment to identify compounds that alter the modification of the NaV by the treatment.

In some embodiments, large compound collections are tested for NaV-modulating activity in a cell-based, functional, high-throughput screen (HTS), e.g., using 96, 384, 1536, or higher density well format. Hits from the HTS screen may be subsequently tested in additional assays to confirm function, e.g., determination of their chemical structures, testing of structurally related compounds to optimize activity and specificity, and further testing in animal models. In some embodiments, the therapeutic potential of modulators is tested in animal models to assess their usefulness in the treatment of human diseases and conditions, including but not limited to epilepsy, periodic paralysis, cardiac diseases, CNS diseases, ataxia, and pain (chronic or acute), loss of ability to feel pain. By way of example, a human NaV 1.7 expressing cell line of the invention can be used to identify a NaV 1.7 antagonist for use as an analgesic to reduce or eliminate pain.

These and other embodiments of the invention may be further illustrated in the following non-limiting Examples.

### EXAMPLES

### Example 1 Generating a Stable NaV 1.7 Heterotrimer-Expressing Cell Line

### Generating Expression Constructs

Plasmid expression vectors that allowed streamlined cloning were generated based on pCMV-SCRIPT (Stratagene) and contained various necessary components for transcription and translation of a gene of interest, including: CMV and SV40 eukaryotic promoters; SV40 and HSV-TK polyadenylation sequences; multiple cloning sites; Kozak sequences; and Neomycin/Kanamycin resistance cassettes (or Ampicillin, Hygromycin, Puromycin, Zeocin resistance cassettes).

### Generation of Cell Lines

293T cells were cotransfected with three separate plasmids, one encoding a human NaV 1.7 α subunit (SEQ ID NO: 13), one encoding a human NaV 1.7 β1 subunit (SEQ ID NO: 16) and one encoding a human NaV 1.7 β2 subunit (SEQ ID NO: 17), using standard techniques. (Examples of reagents that may be used to introduce nucleic acids into host cells include, but are not limited to, LIPOFECTAMINE™, LIPOFECTAMINE™ 2000, OLIGOFECTAMINE™, TFX™ reagents, FUGENE® 6, DOTAP/DOPE, Metafectine or FECTURIN™.)

Although drug selection is optional to produce the cells or cell lines of this invention, we included one drug resistance marker per plasmid. The sequences were under the control of the CMV promoter. An untranslated sequence encoding a Target Sequence for detection by a signaling probe was also present along with the sequence encoding the drug resistance marker. The Target Sequences utilized were Target Sequence 1 (SEQ ID NO: 1), Target Sequence 2 (SEQ ID NO: 2) and Target Sequence 3 (SEQ ID NO: 3). In this example, the NaV 1.7 α subunit gene-containing vector comprised Target Sequence 1 (SEQ ID NO: 1); the NaV 1.7 β1 subunit gene-containing vector comprised Target Sequence 2 (SEQ ID NO: 2); and the NaV 1.7 β2 subunit gene-containing vector comprised Target Sequence 3 (SEQ ID NO: 3).

Transfected cells were grown for 2 days in DMEM-FBS media, followed by 10 days in antibiotic-containing DMEM-FBS media. During the antibiotic containing period, antibiotics were added to the media as follows: puromycin (0.1 μg/ml), hygromycin (100 μg/ml), and zeocin (200 μg/ml).

Following enrichment on antibiotic, cells were passaged 6-18 times in the absence of antibiotic selection to allow time for expression that was not stable over the selected period of time to subside.

Cells were harvested and transfected with signaling probes (SEQ ID NOS: 4, 5, 34) using standard techniques. (Examples of reagents that may be used to introduce nucleic acids into host cells include, but are not limited to, LIPOFECTAMINE™, LIPOFECTAMINE™ 2000, OLIGOFECTAMINE™, TFX™ reagents, FUGENE® 6, DOTAP/DOPE, Metafectine or FECTURIN™.)

Signaling Probe 1(SEQ ID NO: 4) bound Target Sequence 1 (SEQ ID NO: 1); Signaling Probe 2 (SEQ ID NO: 5) bound Target Sequence 2 (SEQ ID NO: 2); and Signaling Probe 3 (SEQ ID NO: 34) bound Target Sequence 3 (SEQ ID NO: 3). The cells were then dissociated and collected for analysis and sorted using a fluorescence activated cell sorter.

Target Sequences Detected by Signaling Probes

The following tag sequences were used for the NaV 1.7 subunit transgenes.

Target Sequence 1
(SEQ ID NO: 1)
5′-GTTCTTAAGGCACAGGAACTGGGAC-3′
(NaV 1.7 α subunit)
Target Sequence 2
(SEQ ID NO: 2)
5′-GAAGTTAACCCTGTCGTTCTGCGAC-3′
(NaV 1.7 β1 subunit)
Target Sequence 3
(SEQ ID NO: 3)
5′-GTTCTATAGGGTCTGCTTGTCGCTC-3′
(NaV 1.7 β2 subunit)

Signaling Probes

Supplied as 100 μM stocks.

Signaling probe 1—This probe binds target sequence 1.

(SEQ ID NO: 4)
5′-Cy5 GCCAGTCCCAGTTCCTGTGCCTTAAGAACCTCGC
BHQ3 quench-3′

Signaling probe 2—This probe binds target sequence 2.

(SEQ ID NO: 5)
5′-Cy5.5 CGAGTCGCAGAACGACAGGGTTAACTTCCTCGC
BHQ3 quench-3′

Signaling probe 3—This probe binds target sequence 3.

(SEQ ID NO: 34)
5′-Fam CGAGAGCGACAAGCAGACCCTATAGAACCTCGC
BHQ1 quench-3′

BHQ3 in Signaling probes 1 and 2 can be replaced by BHQ2 or gold particle. BHQ1 in Signaling probe 3 can be replaced by BHQ2, gold particle, or DABCYL.

In addition, a similar probe using a Quasar® Dye (BioSearch) with spectral properties similar to Cy5 was used in certain experiments. In some experiments, 5-MedC and 2-amino dA mixmer probes were used rather than DNA probes.

Standard analytical methods were used to gate cells fluorescing above background and to isolate cells falling within the defined gate directly into 96-well plates. Flow cytometric cell sorting was operated such that a single cell was deposited per well. After selection, the cells were expanded in media lacking drug. The following gating hierarchy was used:

coincidence gate→singlets gate→live gate→Sort gate in plot FAM vs. Cy5: 0.1-1.0% of live cells.

The above steps were repeated to obtain a greater number of cells. At least four independent rounds of the above steps were completed, and for each of these rounds, at least two internal cycles of cell passsaging and isolation were performed.

The plates were transferred to a Microlabstar automated liquid handler (Hamilton Robotics). Cells were incubated for 5-7 days in a 1:1 mix of fresh complete growth medium (DMEM/10% FBS) and 2-3 day conditioned growth medium, supplemented with 100 units/ml penicillin and 0.1 mg/ml streptomycin. Then the cells were dispersed by trypsinization to minimize clumps and transferred to new 96-well plates. After the clones were dispersed, plates were imaged to determine confluency of wells (Genetix). Each plate was focused for reliable image acquisition across the plate. Reported confluencies of greater than 70% were not relied upon. Confluency measurements were obtained at days every 3 times over 9 days (i.e, between days 1 and 10 post-dispersal) and used to calculate growth rates.

Cells were binned (independently grouped and plated as a cohort) according to growth rate between 10-11 days following the dispersal step in step 7. Bins were independently collected and plated on individual 96 well plates for downstream handling; some growth bins resulted in more than one 96-well plate. Bins were calculated by considering the spread of growth rates and bracketing a high percentage of the total number of populations of cells. Depending on the sort iteration described in Step 5, between 5 and 9 growth bins were used with a partition of 1-4 days. Therefore, each bin corresponded to a growth rate or population doubling time between 8 and 14.4 hours depending on the iteration.

Cells can have doubling times from less 1 day to more than 2 weeks. In order to process the most diverse clones that at the same time can be reasonably binned according to growth rate, it is preferable to use 3-9 bins with a 0.25 to 0.7 day doubling time per bin. One skilled in the art will appreciate that the tightness of the bins and number of bins can be adjusted for the particular situation and that the tightness and number of bins can be further adjusted if cells are synchronized for their cell cycle.

The plates were incubated under standard and fixed conditions (humidified 37° C., 5% CO2) in antibiotics-free DMEM-10% FBS media. The plates of cells were split to produce 4 sets of target plates. These 4 sets of plates comprised all plates with all growth bins to ensure there were 4 replicates of the initial set. Up to 3 target plate sets were committed for cryopreservation (described in step 10), and the remaining set was scaled and further replica plated for passage and functional assay experiments. Distinct and independent tissue culture reagents, incubators, personnel, and carbon dioxide sources were used for downstream replica plates. Quality control steps were taken to ensure the proper production and quality of all tissue culture reagents: each component added to each bottle of media prepared for use was added by one designated person in one designated hood with only that reagent in the hood while a second designated person monitored to avoid mistakes. Conditions for liquid handling were set to eliminate cross contamination across wells. Fresh tips were used for all steps, or stringent tip washing protocols were used. Liquid handling conditions were set for accurate volume transfer, efficient cell manipulation, washing cycles, pipetting speeds and locations, number of pipetting cycles for cell dispersal, and relative position of tip to plate.

Three sets of plates were frozen at −70 to −80° C. Plates in each set were first allowed to attain confluencies of 70 to 80%. Medium was aspirated and 90% FBS and 5%-10% DMSO was added. The plates were sealed with Parafilm, individually surrounded by 1 to 5 cm of foam, and then placed into a −80° C. freezer.

The remaining set of plates was maintained as described in step 9. All cell splitting was performed using automated liquid handling steps, including media removal, cell washing, trypsin addition and incubation, quenching and cell dispersal steps. For some assay plating steps, cells were dissociated with cell dissociation buffer (e.g., CDB, Invitrogen or CellStripper, CellGro) rather than trypsin.

The consistency and standardization of cell and culture conditions for all populations of cells was controlled. Differences across plates due to slight differences in growth rates were controlled by periodic normalization of cell numbers across plates every 2 to 8 passages. Populations of cells that were outliers were detected and eliminated.

The cells were maintained for 3 to 8 weeks to allow for their in vitro evolution under these conditions. During this time, we observed size, morphology, fragility, response to trypsinization or dissociation, roundness/average circularity post-dissociation, percentage viability, tendency towards microconfluency, or other aspects of cell maintenance such as adherence to culture plate surfaces.

Populations of cells were tested using functional criteria. Membrane potential assay kits (Molecular Devices/MDS) were used according to manufacturer's instructions. Cells were tested at multiple different densities in 96- or 384-well plates and responses were analyzed. A variety of post-plating time points were used, e.g., 12-48 hours post plating. Different densities of plating were also tested for assay response differences.

The functional responses from experiments performed at low and higher passage numbers were compared to identify cells with the most consistent responses over defined periods of time, ranging from 3 to 9 weeks. Other characteristics of the cells that changed over time were also noted.

Populations of cells meeting functional and other criteria were further evaluated to determine those most amenable to production of viable, stable and functional cell lines. Selected populations of cells were expanded in larger tissue culture vessels and the characterization steps described above were continued or repeated under these conditions. At this point, additional standardization steps, such as different plating cell densities; time of passage; culture dish size/format and coating); fluidics optimization; cell dissociation optimization (e.g., type, volume used, and length of time); and washing steps, were introduced for consistent and reliable passages. Temperature differences were also used for standardization (i.e., 30° C. vs 37° C.).

In addition, viability of cells at each passage was determined. Manual intervention was increased and cells were more closely observed and monitored. This information was used to help identify and select final cell lines that retained the desired properties. Final cell lines and back-up cell lines were selected that showed consistent growth, appropriate adherence, and functional response.

The low passage frozen plates described above corresponding to the final cell line and back-up cell lines were thawed at 37° C., washed two times with DMEM-10% FBS and incubated in humidified 37° C./5% CO2 conditions. The cells were then expanded for a period of 2-3 weeks. Cell banks for each final and back-up cell line consisting of 15-20 vials were established.

The following step can also be conducted to confirm that the cell lines are viable, stable, and functional. At least one vial from the cell bank is thawed and expanded in culture. The resulting cells are tested to determine if they meet the same characteristics for which they were originally selected.

### Example 2 Characterizing Relative Expression of Heterologous NaV 1.7 Subunits in Stable NaV 1.7-Expressing Cell Lines

Quantitative RT-PCR (qRT-PCR) was used to determine the relative expression of the heterologous human NaV 1.7 α, β1, and β2 subunits in the produced stable NaV 1.7-expressing cell lines. Total RNA was purified from 1-3×10⁶ mammalian cells using an RNA extraction kit (RNeasy Mini Kit, Qiagen). DNase treatment was done according to rigorous DNase treatment protocol (TURBO DNA-free Kit, Ambion). First strand cDNA synthesis was performed using a reverse transcriptase kit (SuperScript III, Invitrogen) in 20 μL reaction volume with 1 μg DNA-free total RNA and 250 ng Random Primers (Invitrogen). Samples without reverse transcriptase and sample without RNA were used as negative controls for this reaction. Synthesis was done in a thermal cycler (Mastercycler, Eppendorf) at the following conditions: 5 min at 25° C., 60 min at 50° C.; reaction termination was conducted for 15 min at 70° C.

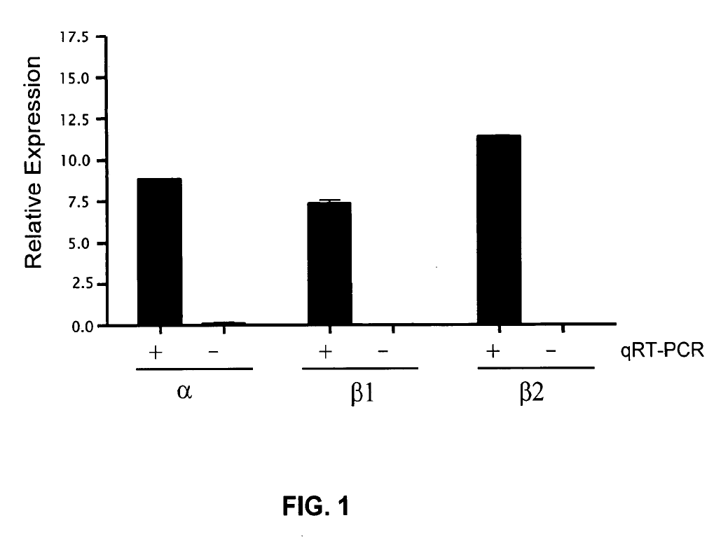

For analysis of gene expression, primers and probes for qRT-PCR (MGB TaqMan probes, Applied Biosystems) were designed to specifically anneal to the target sequences (SEQ ID NOS: 1-3). For sample normalization, control (glyceraldehyde 3-phosphate dehydrogenase (GAPDH)) Pre-Developed Assay reagents (TaqMaN, Applied Biosystems) were used. Reactions, including negative controls and positive controls (plasmid DNA), were set up in triplicates with 40 ng of cDNA in 50 μL reaction volume. The relative amounts of each of the three NaV 1.7 subunits being expressed were determined. As shown in FIG. 1, all three subunits were successfully expressed in the produced stable NaV 1.7-expressing cell line.

### Example 3 Characterizing Stable NaV 1.7-Expressing Cell Lines for Native NaV Function Using Electrophysiological Assay

Automated patch-clamp system was used to record sodium currents from the produced stable HEK293T cell lines expressing NaV 1.7 α, β1, and β2 subunits. The following illustrated protocol can also be used for QPatch, Sophion or Patchliner, Nanion systems. The extracellular Ringer's solution contained 140 mM NaCl, 4.7 mM KCl, 2.6 mM MgCl₂, 11 mM glucose and 5 mM HEPES, pH 7.4 at room temperature. The intracellular Ringer's solution contained 120 mM CsF, 20 mM Cs-EGTA, 1 mM CaCl₂, 1 mM MgCl₂, and 10 mM HEPES, pH 7.2. Experiments were conducted at room temperature.

Cells stably expressing NaV 1.7 α, β1, and β2 subunits were grown under standard culturing protocols as described in Example 1. Cells were harvested and kept in suspension with continuous stirring for up to 4 hours with no significant change in quality or ability to patch. Electrophysiological experiment (whole-cell) was performed using the standard patch plate. The patch-clamp hole (micro-etched in the chip) is approximately 1 μm in diameter and has a resistance of ˜2 MΩ. The membrane potential was clamped to a holding potential of −100 mV.

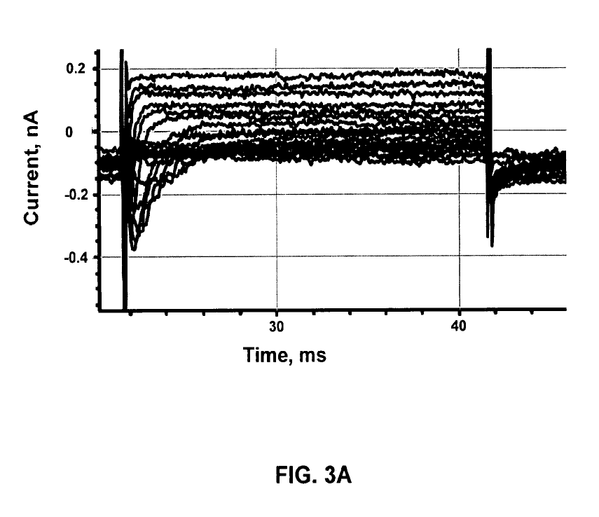

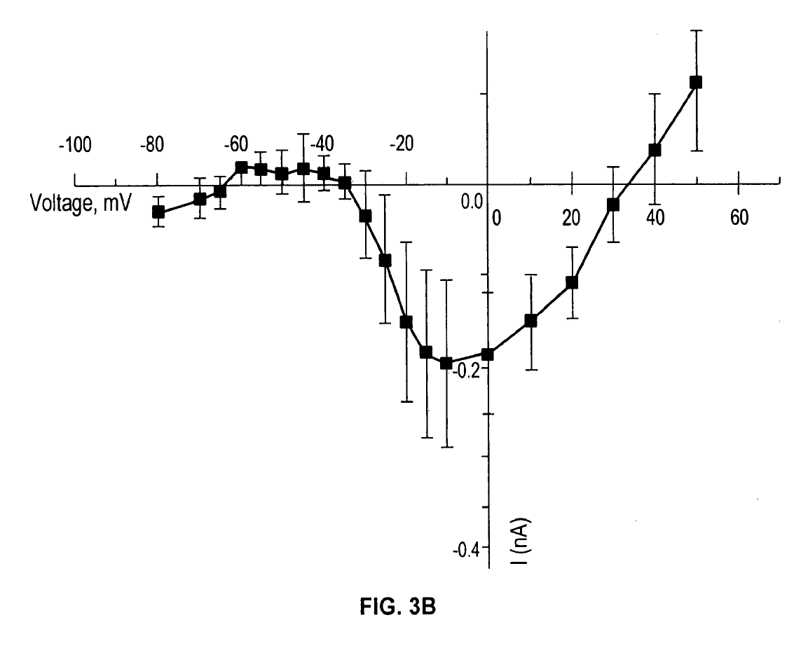

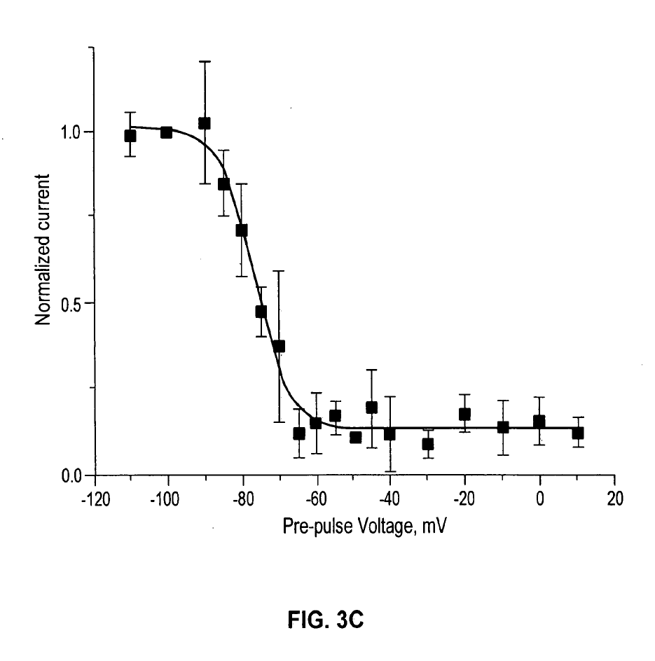

Current-voltage relation and inactivation characteristics of voltage-gated human NaV 1.7 sodium channel stably expressed in HEK293T cells are shown on FIGS. 3A-C. FIG. 3A shows sodium currents in response to 20 ms depolarization pulses from −80 mV to +50 mV. The holding potential was −100 mV. FIG. 3B shows the resulting current-voltage (I-V) relationship for peak sodium channel currents. The activation threshold was −35 mV (midpoint of activation, Va=−24.9 mV+/−3.7 mV), and the maximal current amplitude was obtained at −10 mV. FIG. 3C shows the inactivation graph for the sodium channel. The membrane potential was held at a holding potential of −100 mV, subsequently shifted to conditioning potentials ranging from −110 mV to +10 mV for 1000 ms, and finally the current was measured upon a step to 0 mV. The resulting current amplitude indicates the fraction of sodium channels in the inactivated state. At potentials more negative than −85 mV the channels were predominantly in the closed state, whereas at potentials above −50 mV they were predominantly in the inactivated state. The curve represents the Boltzmann fit from which the V_{1/2} for steady-state inactivation was estimated to be −74 mV. The current-voltage profile for the produced stable NaV 1.7-expressing cell lines is consistent with previously reported current-voltage profile (Va=−28.0 mV±1.1 mV; V_{1/2}=−71.3 mV±0.8 mV) (Sheets et al., J Physiol. 581(Pt 3):1019-1031. (2007)).

### Example 4 Characterizing Stable NaV 1.7-Expressing Cell Lines for Native NaV Function Using Membrane Potential Assay

The produced stable cells expressing NaV 1.7 α, β1, and β2 subunits were maintained under standard cell culture conditions in Dulbecco's Modified Eagles medium supplemented with 10% fetal bovine serum, glutamine and HEPES. On the day before assay, the cells were harvested from stock plates using cell dissociation buffer, e.g., CDB (GIBCO) or cell-stripper (Mediatech), and plated at 10,000-25,000 cells per well in 384 well plates in growth media. The assay plates were maintained in a 37° C. cell culture incubator under 5% CO2 for 22-24 hours. The media were then removed from the assay plates and blue fluorescence membrane potential dye (Molecular Devices Inc.) diluted in load buffer (137 mM NaCl, 5 mM KCl, 1.25 mM CaCl₂, 25 mM HEPES, 10 mM glucose) was added. The cells were incubated with blue membrane potential dye for 1 hour at 37° C. The assay plates were then loaded onto the high-throughput fluorescent plate reader (Hamamastu FDSS). The fluorescent plate reader measures cell fluorescence in images taken of the cell plate once per second and displays the data as relative florescence units.

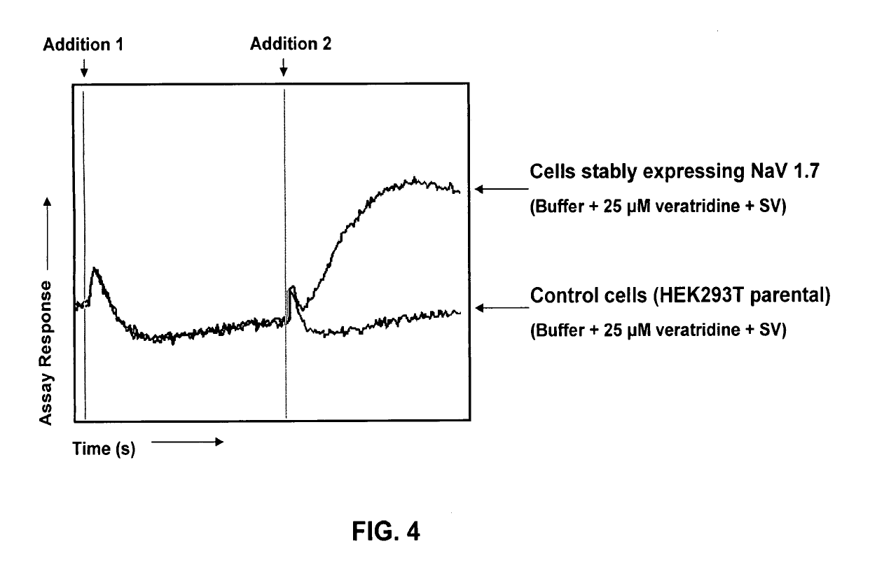

FIG. 4 demonstrates the assay response of stable NaV 1.7-expressing cells and control cells (i.e., HEK293T parental cells) to addition of buffer and channel activators (i.e., veratridine and scorpion venom (SV)). In a first addition step (Addition 1 in FIG. 4), only buffer was added, with no test compounds added. If desired, test compounds can be added in this step. In a second addition step, veratridine and scorpion venom, which are sodium channels activators, were diluted in assay buffer to the desired concentration (i.e., 25 μM veratridine and 5-25 μg/ml scorpion venom) and added into 384 well polypropylene microtiter plates. Once bound, veratridine and scorpion venom proteins modulate the activity of voltage-gated sodium channels through a combination of mechanisms, including an alteration of the activation and inactivation kinetics. The resulted activation of sodium channels in stable NaV 1.7-expressing cells changes cells membrane potential and the fluorescent signal increases. The above-described functional assay can also be used to characterize the relative potencies of test compounds at NaV 1.7 ion channels.

### Example 5 Characterizing Regulation of NaV 1.7 Alpha Subunit by Beta Subunits

### Regulation of Alpha Subunit Gene Expression by Beta Subunits

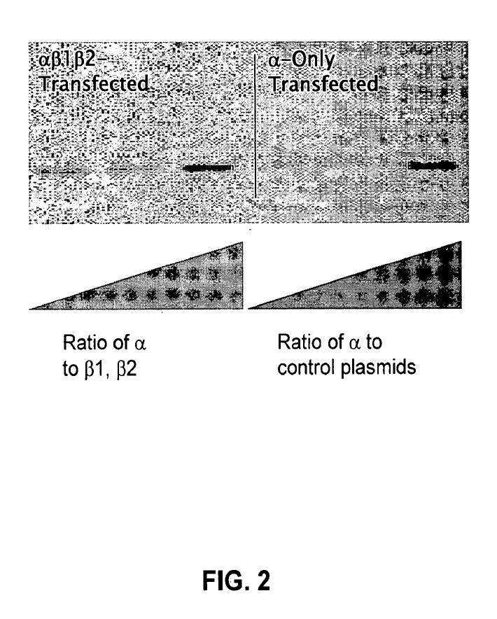

Pools of HEK293T cells were engineered to express various ratios of α and β subunits by manipulating the molar ratios of independent plasmid DNAs or α and control plasmids (e.g., α:β1:β2=1:1:1). After drug selection the subunits expression in six different cell pools were evaluated with qRT-PCR as described in Example 2. Comparative qRT-PCR indicated that α subunit expression in drug-selected cells detection was increased when all three human NaV 1.7 subunits (i.e., α, β1, and β2) were co-transfected (FIG. 2, left panel) in compared to only α subunit and control plasmid transfected (FIG. 2, right panel). The presence of the β subunit transcripts affects α subunit gene expression, demonstrating the importance of co-expressing all three NaV 1.7 subunits for a physiologically relevant functional assay.

### Regulation of Pharmacological Properties by Beta Subunits

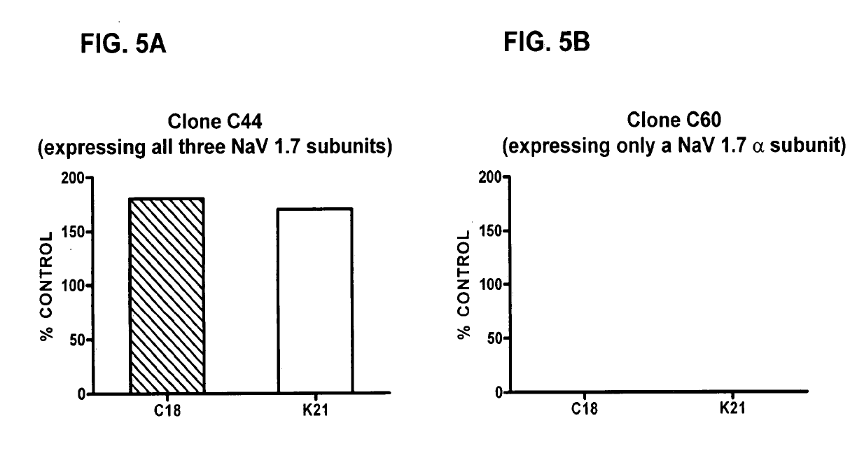

A membrane potential cell-based assay was used to measure the response to test compounds of the cells stably co-expressing all three NaV 1.7 subunits (i.e., α, β1, and β2) and control cells stably expressing only a NaV 1.7 α subunit. Two compounds (FIG. 5) (i.e., C18 and K21) were tested in the membrane potential assay performed substantially according to the protocol in Example 4. Specifically for this example, the test compounds were added in the first addition step.

As shown in FIG. 5, C18 and K21 potentiated the response of clone C44 (expressing NaV 1.7 α, β1, and β2 subunits, FIG. 5A) and blocked the response of clone C60 (expressing NaV 1.7 α subunit only, FIG. 5B). The assay response of the two test compounds was normalized to the response of buffer alone for each of the two clones.

LISTING OF SEQUENCES

Target sequence 1
(SEQ ID NO: 1)
5′-GTTCTTAAGGCACAGGAACTGGGAC-3′
Target sequence 2
(SEQ ID NO: 2)
5′-GAAGTTAACCCTGTCGTTCTGCGAC-3′
Target sequence 3
(SEQ ID NO: 3)
5′-GTTCTATAGGGTCTGCTTGTCGCTC-3′
Signaling probe 1 (binds target 1)
(SEQ ID NO: 4)
5′-Cy5 GCCAGTCCCAGTTCCTGTGCCTTAAGAACCTCGC BHQ3
quench-3′
Signaling probe 2- (binds target 2)
(SEQ ID NO: 5)
5′-Cy5.5 GCGAGTCGCAGAACGACAGGGTTAACTTCCTCGC BHQ3
quench-3′
Homo sapiens (H.s.) SCN1A
(SEQ ID NO: 6)
atggagcaaacagtgcttgtaccaccaggacctgacagcttcaacttct
tcaccagagaatctcttgcggctattgaaagacgcattgcagaagaaaa
ggcaaagaatcccaaaccagacaaaaaagatgacgacgaaaatggccca
aagccaaatagtgacttggaagctggaaagaaccttccatttatttatg
gagacattcctccagagatggtgtcagagcccctggaggacctggaccc
ctactatatcaataagaaaacttttatagtattgaataaagggaaggcc
atcttccggttcagtgccacctctgccctgtacattttaactcccttca
atcctcttaggaaaatagctattaagattttggtacattcattattcag
catgctaattatgtgcactattttgacaaactgtgtgtttatgacaatg
agtaaccctcctgattggacaaagaatgtagaatacaccttcacaggaa
tatatacttttgaatcacttataaaaattattgcaaggggattctgttt
agaagattttactttccttcgggatccatggaactggctcgatttcact
gtcattacatttgcgtacgtcacagagtttgtggacctgggcaatgtct
cggcattgagaacattcagagttctccgagcattgaagacgatttcagt
cattccaggcctgaaaaccattgtgggagccctgatccagtctgtgaag
aagctctcagatgtaatgatcctgactgtgttctgtctgagcgtatttg
ctctaattgggctgcagctgttcatgggcaacctgaggaataaatgtat
acaatggcctcccaccaatgcttccttggaggaacatagtatagaaaag
aatataactgtgaattataatggtacacttataaatgaaactgtctttg
agtttgactggaagtcatatattcaagattcaagatatcattatttcct
ggagggttttttagatgcactactatgtggaaatagctctgatgcaggc
caatgtccagagggatatatgtgtgtgaaagctggtagaaatcccaatt
atggctacacaagctttgataccttcagttgggcttttttgtccttgtt
tcgactaatgactcaggacttctgggaaaatctttatcaactgacatta
cgtgctgctgggaaaacgtacatgatattttttgtattggtcattttct
tgggctcattctacctaataaatttgatcctggctgtggtggccatggc
ctacgaggaacagaatcaggccaccttggaagaagcagaacagaaagag
gccgaatttcagcagatgattgaacagcttaaaaagcaacaggaggcag
ctcagcaggcagcaacggcaactgcctcagaacattccagagagcccag
tgcagcaggcaggctctcagacagctcatctgaagcctctaagttgagt
tccaagagtgctaaggaaagaagaaatcggaggaagaaaagaaaacaga
aagagcagtctggtggggaagagaaagatgaggatgaattccaaaaatc
tgaatctgaggacagcatcaggaggaaaggttttcgcttctccattgaa
gggaaccgattgacatatgaaaagaggtactcctccccacaccagtctt
tgttgagcatccgtggctccctattttcaccaaggcgaaatagcagaac
aagccttttcagctttagagggcgagcaaaggatgtgggatctgagaac
gacttcgcagatgatgagcacagcacctttgaggataacgagagccgta
gagattccttgtttgtgccccgacgacacggagagagacgcaacagcaa
cctgagtcagaccagtaggtcatcccggatgctggcagtgtttccagcg
aatgggaagatgcacagcactgtggattgcaatggtgtggtttccttgg
ttggtggaccttcagttcctacatcgcctgttggacagcttctgccaga
gggaacaaccactgaaactgaaatgagaaagagaaggtcaagttctttc
cacgtttccatggactttctagaagatccttcccaaaggcaacgagcaa
tgagtatagccagcattctaacaaatacagtagaagaacttgaagaatc
caggcagaaatgcccaccctgttggtataaattttccaacatattctta
atctgggactgttctccatattggttaaaagtgaaacatgttgtcaacc
tggttgtgatggacccatttgttgacctggccatcaccatctgtattgt
cttaaatactcttttcatggccatggagcactatccaatgacggaccat
ttcaataatgtgcttacagtaggaaacttggttttcactgggatcttta
cagcagaaatgtttctgaaaattattgccatggatccttactattattt
ccaagaaggctggaatatctttgacggttttattgtgacgcttagcctg
gtagaacttggactcgccaatgtggaaggattatctgttctccgttcat
ttcgattgctgcgagttttcaagttggcaaaatcttggccaacgttaaa
tatgctaataaagatcatcggcaattccgtgggggctctgggaaattta
accctcgtcttggccatcatcgtcttcatttttgccgtggtcggcatgc
agctctttggtaaaagctacaaagattgtgtctgcaagatcgccagtga
ttgtcaactcccacgctggcacatgaatgacttcttccactccttcctg
attgtgttccgcgtgctgtgtggggagtggatagagaccatgtgggact
gtatggaggttgctggtcaagccatgtgccttactgtcttcatgatggt
catggtgattggaaacctagtggtcctgaatctctttctggccttgctt
ctgagctcatttagtgcagacaaccttgcagccactgatgatgataatg
aaatgaataatctccaaattgctgtggataggatgcacaaaggagtagc
ttatgtgaaaagaaaaatatatgaatttattcaacagtccttcattagg
aaacaaaagattttagatgaaattaaaccacttgatgatctaaacaaca
agaaagacagttgtatgtccaatcatacagcagaaattgggaaagatct
tgactatcttaaagatgtaaatggaactacaagtggtataggaactggc
agcagtgttgaaaaatacattattgatgaaagtgattacatgtcattca
taaacaaccccagtcttactgtgactgtaccaattgctgtaggagaatc
tgactttgaaaatttaaacacggaagactttagtagtgaatcggatctg
gaagaaagcaaagagaaactgaatgaaagcagtagctcatcagaaggta
gcactgtggacatcggcgcacctgtagaagaacagcccgtagtggaacc
tgaagaaactcttgaaccagaagcttgtttcactgaaggctgtgtacaa
agattcaagtgttgtcaaatcaatgtggaagaaggcagaggaaaacaat
ggtggaacctgagaaggacgtgtttccgaatagttgaacataactggtt
tgagaccttcattgttttcatgattctccttagtagtggtgctctggca
tttgaagatatatatattgatcagcgaaagacgattaagacgatgttgg
aatatgctgacaaggttttcacttacattttcattctggaaatgcttct
aaaatgggtggcatatggctatcaaacatatttcaccaatgcctggtgt
tggctggacttcttaattgttgatgtttcattggtcagtttaacagcaa
atgccttgggttactcagaacttggagccatcaaatctctcaggacact
aagagctctgagacctctaagagccttatctcgatttgaagggatgagg
gtggttgtgaatgcccttttaggagcaattccatccatcatgaatgtgc
ttctggtttgtcttatattctggctaattttcagcatcatgggcgtaaa
tttgtttgctggcaaattctaccactgtattaacaccacaactggtgac
aggtttgacatcgaagacgtgaataatcatactgattgcctaaaactaa
tagaaagaaatgagactgctcgatggaaaaatgtgaaagtaaactttga
taatgtaggatttgggtatctctctttgcttcaagttgccacattcaaa
ggatggatggatataatgtatgcagcagttgattccagaaatgtggaac
tccagcctaagtatgaagaaagtctgtacatgtatctttactttgttat
tttcatcatctttgggtccttcttcaccttgaacctgtttattggtgtc
atcatagataatttcaaccagcagaaaaagaagtttggaggtcaagaca
tctttatgacagaagaacagaagaaatactataatgcaatgaaaaaatt
aggatcgaaaaaaccgcaaaagcctatacctcgaccaggaaacaaattt
caaggaatggtctttgacttcgtaaccagacaagtttttgacataagca
tcatgattctcatctgtcttaacatggtcacaatgatggtggaaacaga
tgaccagagtgaatatgtgactaccattttgtcacgcatcaatctggtg
ttcattgtgctatttactggagagtgtgtactgaaactcatctctctac
gccattattattttaccattggatggaatatttttgattttgtggttgt
cattctctccattgtaggtatgtttcttgccgagctgatagaaaagtat
ttcgtgtcccctaccctgttccgagtgatccgtcttgctaggattggcc
gaatcctacgtctgatcaaaggagcaaaggggatccgcacgctgctctt
tgctttgatgatgtcccttcctgcgttgtttaacatcggcctcctactc
ttcctagtcatgttcatctacgccatctttgggatgtccaactttgcct
atgttaagagggaagttgggatcgatgacatgttcaactttgagacctt
tggcaacagcatgatctgcctattccaaattacaacctctgctggctgg
gatggattgctagcacccattctcaacagtaagccacccgactgtgacc
ctaataaagttaaccctggaagctcagttaagggagactgtgggaaccc
atctgttggaattttcttttttgtcagttacatcatcatatccttcctg
gttgtggtgaacatgtacatcgcggtcatcctggagaacttcagtgttg
ctactgaagaaagtgcagagcctctgagtgaggatgactttgagatgtt
ctatgaggtttgggagaagtttgatcccgatgcaactcagttcatggaa
tttgaaaaattatctcagtttgcagctgcgcttgaaccgcctctcaatc
tgccacaaccaaacaaactccagctcattgccatggatttgcccatggt
gagtggtgaccggatccactgtcttgatatcttatttgcttttacaaag
cgggttctaggagagagtggagagatggatgctctacgaatacagatgg
aagagcgattcatggcttccaatccttccaaggtctcctatcagccaat
cactactactttaaaacgaaaacaagaggaagtatctgctgtcattatt
cagcgtgcttacagacgccaccttttaaagcgaactgtaaaacaagctt
cctttacgtacaataaaaacaaaatcaaaggtggggctaatcttcttat
aaaagaagacatgataattgacagaataaatgaaaactctattacagaa
aaaactgatctgaccatgtccactgcagcttgtccaccttcctatgacc
gggtgacaaagccaattgtggaaaaacatgagcaagaaggcaaagatga
aaaagccaaagggaaataa
H.s. SCN2A
(SEQ ID NO: 7)
atggcacagtcagtgctggtaccgccaggacctgacagcttccgcttct
ttaccagggaatcccttgctgctattgaacaacgcattgcagaagagaa
agctaagagacccaaacaggaacgcaaggatgaggatgatgaaaatggc
ccaaagccaaacagtgacttggaagcaggaaaatctcttccatttattt
atggagacattcctccagagatggtgtcagtgcccctggaggatctgga
cccctactatatcaataagaaaacgtttatagtattgaataaagggaaa
gcaatctctcgattcagtgccacccctgccctttacattttaactccct
tcaaccctattagaaaattagctattaagattttggtacattctttatt
caatatgctcattatgtgcacgattcttaccaactgtgtatttatgacc
atgagtaaccctccagactggacaaagaatgtggagtatacctttacag
gaatttatacttttgaatcacttattaaaatacttgcaaggggcttttg
tttagaagatttcacatttttacgggatccatggaattggttggatttc
acagtcattacttttgcatatgtgacagagtttgtggacctgggcaatg
tctcagcgttgagaacattcagagttctccgagcattgaaaacaatttc
agtcattccaggcctgaagaccattgtgggggccctgatccagtcagtg
aagaagctttctgatgtcatgatcttgactgtgttctgtctaagcgtgt
ttgcgctaataggattgcagttgttcatgggcaacctacgaaataaatg
tttgcaatggcctccagataattcttcctttgaaataaatatcacttcc
ttctttaacaattcattggatgggaatggtactactttcaataggacag
tgagcatatttaactgggatgaatatattgaggataaaagtcactttta
ttttttagaggggcaaaatgatgctctgctttgtggcaacagctcagat
gcaggccagtgtcctgaaggatacatctgtgtgaaggctggtagaaacc
ccaactatggctacacgagctttgacacctttagttgggcctttttgtc
cttatttcgtctcatgactcaagacttctgggaaaacctttatcaactg
acactacgtgctgctgggaaaacgtacatgatattttttgtgctggtca
ttttcttgggctcattctatctaataaatttgatcttggctgtggtggc
catggcctatgaggaacagaatcaggccacattggaagaggctgaacag
aaggaagctgaatttcagcagatgctcgaacagttgaaaaagcaacaag
aagaagctcaggcggcagctgcagccgcatctgctgaatcaagagactt
cagtggtgctggtgggataggagttttttcagagagttcttcagtagca
tctaagttgagctccaaaagtgaaaaagagctgaaaaacagaagaaaga
aaaagaaacagaaagaacagtctggagaagaagagaaaaatgacagagt
ccgaaaatcggaatctgaagacagcataagaagaaaaggtttccgtttt
tccttggaaggaagtaggctgacatatgaaaagagattttcttctccac
accagtccttactgagcatccgtggctcccttttctctccaagacgcaa
cagtagggcgagccttttcagcttcagaggtcgagcaaaggacattggc
tctgagaatgactttgctgatgatgagcacagcacctttgaggacaatg
acagccgaagagactctctgttcgtgccgcacagacatggagaacggcg
ccacagcaatgtcagccaggccagccgtgcctccagggtgctccccatc
ctgcccatgaatgggaagatgcatagcgctgtggactgcaatggtgtgg
tctccctggtcgggggcccttctaccctcacatctgctgggcagctcct
accagagggcacaactactgaaacagaaataagaaagagacggtccagt
tcttatcatgtttccatggatttattggaagatcctacatcaaggcaaa
gagcaatgagtatagccagtattttgaccaacaccatggaagaacttga
agaatccagacagaaatgcccaccatgctggtataaatttgctaatatg
tgtttgatttgggactgttgtaaaccatggttaaaggtgaaacaccttg
tcaacctggttgtaatggacccatttgttgacctggccatcaccatctg
cattgtcttaaatacactcttcatggctatggagcactatcccatgacg
gagcagttcagcagtgtactgtctgttggaaacctggtcttcacaggga
tcttcacagcagaaatgtttctcaagataattgccatggatccatatta
ttactttcaagaaggctggaatatttttgatggttttattgtgagcctt
agtttaatggaacttggtttggcaaatgtggaaggattgtcagttctcc
gatcattccggctgctccgagttttcaagttggcaaaatcttggccaac
tctaaatatgctaattaagatcattggcaattctgtgggggctctagga
aacctcaccttggtattggccatcatcgtcttcatttttgctgtggtcg
gcatgcagctctttggtaagagctacaaagaatgtgtctgcaagatttc
caatgattgtgaactcccacgctggcacatgcatgactttttccactcc
ttcctgatcgtgttccgcgtgctgtgtggagagtggatagagaccatgt
gggactgtatggaggtcgctggccaaaccatgtgccttactgtcttcat
gatggtcatggtgattggaaatctagtggttctgaacctcttcttggcc
ttgcttttgagttccttcagttctgacaatcttgctgccactgatgatg
ataacgaaatgaataatctccagattgctgtgggaaggatgcagaaagg
aatcgattttgttaaaagaaaaatacgtgaatttattcagaaagccttt
gttaggaagcagaaagctttagatgaaattaaaccgcttgaagatctaa
ataataaaaaagacagctgtatttccaaccataccaccatagaaatagg
caaagacctcaattatctcaaagacggaaatggaactactagtggcata
ggcagcagtgtagaaaaatatgtcgtggatgaaagtgattacatgtcat
ttataaacaaccctagcctcactgtgacagtaccaattgctgttggaga
atctgactttgaaaatttaaatactgaagaattcagcagcgagtcagat
atggaggaaagcaaagagaagctaaatgcaactagttcatctgaaggca
gcacggttgatattggagctcccgccgagggagaacagcctgaggttga
acctgaggaatcccttgaacctgaagcctgttttacagaagactgtgta
cggaagttcaagtgttgtcagataagcatagaagaaggcaaagggaaac
tctggtggaatttgaggaaaacatgctataagatagtggagcacaattg
gttcgaaaccttcattgtcttcatgattctgctgagcagtggggctctg
gcctttgaagatatatacattgagcagcgaaaaaccattaagaccatgt
tagaatatgctgacaaggttttcacttacatattcattctggaaatgct
gctaaagtgggttgcatatggttttcaagtgtattttaccaatgcctgg
tgctggctagacttcctgattgttgatgtctcactggttagcttaactg
caaatgccttgggttactcagaacttggtgccatcaaatccctcagaac
actaagagctctgaggccactgagagctttgtcccggtttgaaggaatg
agggttgttgtaaatgctcttttaggagccattccatctatcatgaatg
tacttctggtttgtctgatcttttggctaatattcagtatcatgggagt
gaatctctttgctggcaagttttaccattgtattaattacaccactgga
gagatgtttttgatgtaagcgtggtcaacaactacagtgagtgcaaagc
tctcattgagagcaatcaaactgccaggtggaaaaatgtgaaagtaaac
tttgataacgtaggacttggatatctgtctctacttcaagtagccacgt
ttaagggatggatggatattatgtatgcagctgttgattcacgaaatgt
agaattacaacccaagtatgaagacaacctgtacatgtatctttatttt
gtcatctttattatttttggttcattctttaccttgaatcttttcattg
gtgtcatcatagataacttcaaccaacagaaaaagaagtttggaggtca
agacatttttatgacagaagaacagaagaaatactacaatgcaatgaaa
aaactgggttcaaagaaaccacaaaaacccatacctcgacctgctaaca
aattccaaggaatggtctttgattttgtaaccaaacaagtctttgatat
cagcatcatgatcctcatctgccttaacatggtcaccatgatggtggaa
accgatgaccagagtcaagaaatgacaaacattctgtactggattaatc
tggtgtttattgttctgttcactggagaatgtgtgctgaaactgatctc
tcttcgttactactatttcactattggatggaatatttttgattttgtg
gtggtcattctctccattgtaggaatgtttctggctgaactgatagaaa
agtattttgtgtcccctaccctgttccgagtgatccgtcttgccaggat
tggccgaatcctacgtctgatcaaaggagcaaaggggatccgcacgctg
ctctttgctttgatgatgtcccttcctgcgttgtttaacatcggcctcc
ttcttttcctggtcatgttcatctacgccatctttgggatgtccaattt
tgcctatgttaagagggaagttgggatcgatgacatgttcaactttgag
acctttggcaacagcatgatctgcctgttccaaattacaacctctgctg
gctgggatggattgctagcacctattcttaatagtggacctccagactg
tgaccctgacaaagatcaccctggaagctcagttaaaggagactgtggg
aacccatctgttgggattttcttttttgtcagttacatcatcatatcct
tcctggttgtggtgaacatgtacatcgcggtcatcctggagaacttcag
tgttgctactgaagaaagtgcagagcctctgagtgaggatgactttgag
atgttctatgaggtttgggagaagtttgatcccgatgcgacccagttta
tagagtttgccaaactttctgattttgcagatgccctggatcctcctct
tctcatagcaaaacccaacaaagtccagctcattgccatggatctgccc
atggtgagtggtgaccggatccactgtcttgacatcttatttgctttta
caaagcgtgttttgggtgagagtggagagatggatgcccttcgaataca
gatggaagagcgattcatggcatcaaacccctccaaagtctcttatgag
cccattacgaccacgttgaaacgcaaacaagaggaggtgtctgctatta
ttatccagagggcttacagacgctacctcttgaagcaaaaagttaaaaa
ggtatcaagtatatacaagaaagacaaaggcaaagaatgtgatggaaca
cccatcaaagaagatactctcattgataaactgaatgagaattcaactc
cagagaaaaccgatatgacgccttccaccacgtctccaccctcgtatga
tagtgtgaccaaaccagaaaaagaaaaatttgaaaaagacaaatcagaa
aaggaagacaaagggaaagatatcagggaaagtaaaaagtaa
H.s. SCN3A
(SEQ ID NO: 8)
atggcacaggcactgttggtacccccaggacctgaaagcttccgccttt
ttactagagaatctcttgctgctatcgaaaaacgtgctgcagaagagaa
agccaagaagcccaaaaaggaacaagataatgatgatgagaacaaacca
aagccaaatagtgacttggaagctggaaagaaccttccatttatttatg
gagacattcctccagagatggtgtcagagcccctggaggacctggatcc
ctactatatcaataagaaaacttttatagtaatgaataaaggaaaggca
attttccgattcagtgccacctctgccttgtatattttaactccactaa
accctgttaggaaaattgctatcaagattttggtacattctttattcag
catgcttatcatgtgcactattttgaccaactgtgtatttatgaccttg
agcaaccctcctgactggacaaagaatgtagagtacacattcactggaa
tctatacctttgagtcacttataaaaatcttggcaagagggttttgctt
agaagattttacgtttcttcgtgatccatggaactggctggatttcagt
gtcattgtgatggcatatgtgacagagtttgtggacctgggcaatgtct
cagcgttgagaacattcagagttctccgagcactgaaaacaatttcagt
cattccaggtttaaagaccattgtgggggccctgatccagtcggtaaag
aagctttctgatgtgatgatcctgactgtgttctgtctgagcgtgtttg
ctctcattgggctgcagctgttcatgggcaatctgaggaataaatgttt
gcagtggcccccaagcgattctgcttttgaaaccaacaccacttcctac
tttaatggcacaatggattcaaatgggacatttgttaatgtaacaatga
gcacatttaactggaaggattacattggagatgacagtcacttttatgt
tttggatgggcaaaaagaccctttactctgtggaaatggctcagatgca
ggccagtgtccagaaggatacatctgtgtgaaggctggtcgaaacccca
actatggctacacaagctttgacacctttagctgggctttcctgtctct
atttcgactcatgactcaagactactgggaaaatctttaccagttgaca
ttacgtgctgctgggaaaacatacatgatattttttgtcctggtcattt
tcttgggctcattttatttggtgaatttgatcctggctgtggtggccat
ggcctatgaggagcagaatcaggccaccttggaagaagcagaacaaaaa
gaggccgaatttcagcagatgctcgaacagcttaaaaagcaacaggaag
aagctcaggcagttgcggcagcatcagctgcttcaagagatttcagtgg
aataggtgggttaggagagctgttggaaagttcttcagaagcatcaaag
ttgagttccaaaagtgctaaagaatggaggaaccgaaggaagaaaagaa
gacagagagagcaccttgaaggaaacaacaaaggagagagagacagctt
tcccaaatccgaatctgaagacagcgtcaaaagaagcagcttccttttc
tccatggatggaaacagactgaccagtgacaaaaaattctgctcccctc
atcagtctctcttgagtatccgtggctccctgttttccccaagacgcaa
tagcaaaacaagcattttcagtttcagaggtcgggcaaaggatgttgga
tctgaaaatgactttgctgatgatgaacacagcacatttgaagacagcg
aaagcaggagagactcactgtttgtgccgcacagacatggagagcgacg
caacagtaacgttagtcaggccagtatgtcatccaggatggtgccaggg
cttccagcaaatgggaagatgcacagcactgtggattgcaatggtgtgg
tttccttggtgggtggaccttcagctctaacgtcacctactggacaact
tcccccagagggcaccaccacagaaacggaagtcagaaagagaaggtta
agctcttaccagatttcaatggagatgctggaggattcctctggaaggc
aaagagccgtgagcatagccagcattctgaccaacacaatggaagaact
tgaagaatctagacagaaatgtccgccatgctggtatagatttgccaat
gtgttcttgatctgggactgctgtgatgcatggttaaaagtaaaacatc
ttgtgaatttaattgttatggatccatttgttgatcttgccatcactat
ttgcattgtcttaaataccctctttatggccatggagcactaccccatg
actgagcaattcagtagtgtgttgactgtaggaaacctggtctttactg
ggattttcacagcagaaatggttctcaagatcattgccatggatcctta
ttactatttccaagaaggctggaatatctttgatggaattattgtcagc
ctcagtttaatggagcttggtctgtcaaatgtggagggattgtctgtac
tgcgatcattcagactgcttagagttttcaagttggcaaaatcctggcc
cacactaaatatgctaattaagatcattggcaattctgtgggggctcta
ggaaacctcaccttggtgttggccatcatcgtcttcatttttgctgtgg
tcggcatgcagctctttggtaagagctacaaagaatgtgtctgcaagat
caatgatgactgtacgctcccacggtggcacatgaacgacttcttccac
tccttcctgattgtgttccgcgtgctgtgtggagagtggatagagacca
tgtgggactgtatggaggtcgctggccaaaccatgtgccttattgtttt
catgttggtcatggtcattggaaaccttgtggttctgaacctctttctg
gccttattgttgagttcatttagctcagacaaccttgctgctactgatg
atgacaatgaaatgaataatctgcagattgcagtaggaagaatgcaaaa
gggaattgattatgtgaaaaataagatgcgggagtgtttccaaaaagcc
ttttttagaaagccaaaagttatagaaatccatgaaggcaataagatag
acagctgcatgtccaataatactggaattgaaataagcaaagagcttaa
ttatcttagagatgggaatggaaccaccagtggtgtaggtactggaagc
agtgttgaaaaatacgtaatcgatgaaaatgattatatgtcattcataa
acaaccccagcctcaccgtcacagtgccaattgctgttggagagtctga
ctttgaaaacttaaatactgaagagttcagcagtgagtcagaactagaa
gaaagcaaagagaaattaaatgcaaccagctcatctgaaggaagcacag
ttgatgttgttctaccccgagaaggtgaacaagctgaaactgaacccga
agaagaccttaaaccggaagcttgttttactgaaggatgtattaaaaag
tttccattctgtcaagtaagtacagaagaaggcaaagggaagatctggt
ggaatcttcgaaaaacctgctacagtattgttgagcacaactggtttga
gactttcattgtgttcatgatccttctcagtagtggtgcattggccttt
gaagatatatacattgaacagcgaaagactatcaaaaccatgctagaat
atgctgacaaagtctttacctatatattcattctggaaatgcttctcaa
atgggttgcttatggatttcaaacatatttcactaatgcctggtgctgg
ctagatttcttgatcgttgatgtttctttggttagcctggtagccaatg
ctcttggctactcagaactcggtgccatcaaatcattacggacattaag
agctttaagacctctaagagccttatcccggtttgaaggcatgagggtg
gttgtgaatgctcttgttggagcaattccctctatcatgaatgtgctgt
tggtctgtctcatcttctggttgatctttagcatcatgggtgtgaattt
gtttgctggcaagttctaccactgtgttaacatgacaacgggtaacatg
tttgacattagtgatgttaacaatttgagtgactgtcaggctcttggca
agcaagctcggtggaaaaacgtgaaagtaaactttgataatgttggcgc
tggctatcttgcactgcttcaagtggccacatttaaaggctggatggat
attatgtatgcagctgttgattcacgagatgttaaacttcagcctgtat
atgaagaaaatctgtacatgtatttatactttgtcatctttatcatctt
tgggtcattcttcactctgaatctattcattggtgtcatcatagataac
ttcaaccagcagaaaaagaagtttggaggtcaagacatctttatgacag
aggaacagaaaaaatattacaatgcaatgaagaaacttggatccaagaa
acctcagaaacccatacctcgcccagcaaacaaattccaaggaatggtc
tttgattttgtaaccagacaagtctttgatatcagcatcatgatcctca
tctgcctcaacatggtcaccatgatggtggaaacggatgaccagggcaa
atacatgaccctagttttgtcccggatcaacctagtgttcattgttctg
ttcactggagaatttgtgctgaagctcgtctccctcagacactactact
tcactataggctggaacatctttgactttgtggtggtgattctctccat
tgtaggtatgtttctggctgagatgatagaaaagtattttgtgtcccct
accttgttccgagtgatccgtcttgccaggattggccgaatcctacgtc
tgatcaaaggagcaaaggggatccgcacgctgctctttgctttgatgat
gtcccttcctgcgttgtttaacatcggcctcctgctcttcctggtcatg
tttatctatgccatctttgggatgtccaactttgcctatgttaaaaagg
aagctggaattgatgacatgttcaactttgagacctttggcaacagcat
gatctgcttgttccaaattacaacctctgctggctgggatggattgcta
gcacctattcttaatagtgcaccacccgactgtgaccctgacacaattc
accctggcagctcagttaagggagactgtgggaacccatctgttgggat
tttcttttttgtcagttacatcatcatatccttcctggttgtggtgaac
atgtacatcgcggtcatcctggagaacttcagtgttgctactgaagaaa
gtgcagagcccctgagtgaggatgactttgagatgttctatgaggtttg
ggaaaagtttgatcccgatgcgacccagtttatagagttctctaaactc
tctgattttgcagctgccctggatcctcctcttctcatagcaaaaccca
acaaagtccagcttattgccatggatctgcccatggtcagtggtgaccg
gatccactgtcttgatattttatttgcctttacaaagcgtgttttgggt
gagagtggagagatggatgcccttcgaatacagatggaagacaggttta
tggcatcaaacccctccaaagtctcttatgagcctattacaaccacttt
gaaacgtaaacaagaggaggtgtctgccgctatcattcagcgtaatttc
agatgttatcttttaaagcaaaggttaaaaaatatatcaagtaactata
acaaagaggcaattaaagggaggattgacttacctataaaacaagacat
gattattgacaaactaaatgggaactccactccagaaaaaacagatggg
agttcctctaccacctctcctccttcctatgatagtgtaacaaaaccag
acaaggaaaagtttgagaaagacaaaccagaaaaagaaagcaaaggaaa
agaggtcagagaaaatcaaaagtaa
H.s. SCN4A
(SEQ ID NO: 9)
atggccagaccatctctgtgcaccctggtgcctctgggccctgagtgct
tgcgccccttcacccgggagtcactggcagccatagaacagcgggcggt
ggaggaggaggcccggctgcagcggaataagcagatggagattgaggag
cccgaacggaagccacgaagtgacttggaggctggcaagaacctaccca
tgatctacggagaccccccgccggaggtcatcggcatccccctggagga
cctggatccctactacagcaataagaagaccttcatcgtactcaacaag
ggcaaggccatcttccgcttctccgccacacctgctctctacctgctga
gccccttcagcgtagtcaggcgcggggccatcaaggtgctcatccatgc
gctgttcagcatgttcatcatgatcaccatcttgaccaactgcgtattc
atgaccatgagtgacccgcctccctggtccaagaatgtggagtacacct
tcacagggatctacacctttgagtccctcatcaagatactggcccgagg
cttctgtgtcgacgacttcacattcctccgggacccctggaactggctg
gacttcagtgtcatcatgatggcgtacctgacagagtttgtggacttgg
gcaacatctcagccctgaggaccttccgggtgctgcgggccctcaaaac
catcacggtcatcccagggctgaagacgatcgtgggggccctgatccag
tcggtgaaaaagctgtcggatgtgatgatcctcactgtcttctgcctga
gcgtctttgcgctggtaggactgcagctcttcatgggaaacctgaggca
gaagtgtgtgcgctggcccccgccgttcaacgacaccaacaccacgtgg
tacagcaatgacacgtggtacggcaatgacacatggtatggcaatgaga
tgtggtacggcaatgactcatggtatgccaacgacacgtggaacagcca
tgcaagctgggccaccaacgatacctttgattgggacgcctacatcagt
gatgaagggaacttctacttcctggagggctccaacgatgccctgctct
gtgggaacagcagtgatgctgggcactgccctgagggttatgagtgcat
caagaccgggcggaaccccaactatggctacaccagctatgacaccttc
agctgggccttcttggctctcttccgcctcatgacacaggactattggg
agaacctcttccagctgacccttcgagcagctggcaagacctacatgat
cttcttcgtggtcatcatcttcctgggctctttctacctcatcaatctg
atcctggccgtggtggccatggcatatgccgagcagaatgaggccaccc
tggccgaggataaggagaaagaggaggagtttcagcagatgcttgagaa
gttcaaaaagcaccaggaggagctggagaaggccaaggccgcccaagct
ctggaaggtggggaggcagatggggacccagcccatggcaaagactgca
atggcagcctggacacatcgcaaggggagaagggagccccgaggcagag
cagcagcggagacagcggcatctccgacgccatggaagaactggaagag
gcccaccaaaagtgcccaccatggtggtacaagtgcgcccacaaagtgc
tcatatggaactgctgcgccccgtggctgaagttcaagaacatcatcca
cctgatcgtcatggacccgttcgtggacctgggcatcaccatctgcatc
gtgctcaacaccctcttcatggccatggaacattaccccatgacggagc
actttgacaacgtgctcactgtgggcaacctggtcttcacaggcatctt
cacagcagagatggttctgaagctgattgccatggacccctacgagtat
ttccagcagggttggaatatcttcgacagcatcatcgtcaccctcagcc
tggtagagctaggcctggccaacgtacagggactgtctgtgctacgctc
cttccgtctgctgcgggtcttcaagctggccaagtcgtggccaacgctg
aacatgctcatcaagatcattggcaattcagtgggggcgctgggtaacc
tgacgctggtgctggctatcatcgtgttcatcttcgccgtggtgggcat
gcagctgtttggcaagagctacaaggagtgcgtgtgcaagattgccttg
gactgcaacctgccgcgctggcacatgcatgatttcttccactccttcc
tcatcgtcttccgcatcctgtgcggggagtggatcgagaccatgtggga
ctgcatggaggtggccggccaagccatgtgcctcaccgtcttcctcatg
gtcatggtcatcggcaatcttgtggtcctgaacctgttcctggctctgc
tgctgagctccttcagcgccgacagtctggcagcctcggatgaggatgg
cgagatgaacaacctgcagattgccatcgggcgcatcaagttgggcatc
ggctttgccaaggccttcctcctggggctgctgcatggcaagatcctga
gccccaaggacatcatgctcagcctcggggaggctgacggggccgggga
ggctggagaggcgggggagactgcccccgaggatgagaagaaggagccg
cccgaggaggacctgaagaaggacaatcacatcctgaaccacatgggcc
tggctgacggccccccatccagcctcgagctggaccaccttaacttcat
caacaacccctacctgaccatacaggtgcccatcgcctccgaggagtcc
gacctggagatgcccaccgaggaggaaaccgacactttctcagagcctg
aggatagcaagaagccgccgcagcctctctatgatgggaactcgtccgt
ctgcagcacagctgactacaagccccccgaggaggaccctgaggagcag
gcagaggagaaccccgagggggagcagcctgaggagtgcttcactgagg
cctgcgtgcagcgctggccctgcctctacgtggacatctcccagggccg
tgggaagaagtggtggactctgcgcagggcctgcttcaagattgtcgag
cacaactggttcgagaccttcattgtcttcatgatcctgctcagcagtg
gggctctggccttcgaggacatctacattgagcagcggcgagtcattcg
caccatcctagaatatgccgacaaggtcttcacctacatcttcatcatg
gagatgctgctcaaatgggtggcctacggctttaaggtgtacttcacca
acgcctggtgctggctcgacttcctcatcgtggatgtctccatcatcag
cttggtggccaactggctgggctactcggagctgggacccatcaaatcc
ctgcggacactgcgggccctgcgtcccctgagggcactgtcccgattcg
agggcatgagggtggtggtgaacgccctcctaggcgccatcccctccat
catgaatgtgctgcttgtctgcctcatcttctggctgatcttcagcatc
atgggtgtcaacctgtttgccggcaagttctactactgcatcaacacca
ccacctctgagaggttcgacatctccgaggtcaacaacaagtctgagtg
cgagagcctcatgcacacaggccaggtccgctggctcaatgtcaaggtc
aactacgacaacgtgggtctgggctacctctccctcctgcaggtggcca
ccttcaagggttggatggacatcatgtatgcagccgtggactcccggga
gaaggaggagcagccgcagtacgaggtgaacctctacatgtacctctac
tttgtcatcttcatcatctttggctccttcttcaccctcaacctcttca
ttggcgtcatcattgacaacttcaaccagcagaagaagaagttaggggg
gaaagacatctttatgacggaggaacagaagaaatactataacgccatg
aagaagcttggctccaagaagcctcagaagccaattccccggccccaga
acaagatccagggcatggtgtatgacctcgtgacgaagcaggccttcga
catcaccatcatgatcctcatctgcctcaacatggtcaccatgatggtg
gagacagacaaccagagccagctcaaggtggacatcctgtacaacatca
acatgatcttcatcatcatcttcacaggggagtgcgtgctcaagatgct
cgccctgcgccagtactacttcaccgttggctggaacatctttgacttc
gtggtcgtcatcctgtccattgtgggccttgccctctctgacctgatcc
agaagtacttcgtgtcacccacgctgttccgtgtgatccgcctggcgcg
gattgggcgtgtcctgcggctgatccgcggggccaagggcatccggacg
ctgctgttcgccctcatgatgtcgctgcctgccctcttcaacatcggcc
tcctcctcttcctggtcatgttcatctactccatcttcggcatgtccaa
ctttgcctacgtcaagaaggagtcgggcatcgatgatatgttcaacttc
gagaccttcggcaacagcatcatctgcctgttcgagatcaccacgtcgg
ccggctgggacgggctcctcaaccccatcctcaacagcgggcccccaga
ctgtgaccccaacctggagaacccgggcaccagtgtcaagggtgactgc
ggcaacccctccatcggcatctgcttcttctgcagctatatcatcatct
ccttcctcatcgtggtcaacatgtacatcgccatcatcctggagaactt
caatgtggccacagaggagagcagcgagccccttggtgaagatgacttt
gagatgttctacgagacatgggagaagttcgaccccgacgccacccagt
tcatcgcctacagccgcctctcagacttcgtggacaccctgcaggaacc
gctgaggattgccaagcccaacaagatcaagctcatcacactggacttg
cccatggtgccaggggacaagatccactgcctggacatcctctttgccc
tgaccaaagaggtcctgggtgactctggggaaatggacgccctcaagca
gaccatggaggagaagttcatggcagccaacccctccaaggtgtcctac
gagcccatcaccaccaccctcaagaggaagcacgaggaggtgtgcgcca
tcaagatccagagggcctaccgccggcacctgctacagcgctccatgaa
gcaggcatcctacatgtaccgccacagccacgacggcagcggggatgac
gcccctgagaaggaggggctgcttgccaacaccatgagcaagatgtatg
gccacgagaatgggaacagcagctcgccaagcccggaggagaagggcga
ggcaggggacgccggacccactatggggctgatgcccatcagcccctca
gacactgcctggcctcccgcccctcccccagggcagactgtgcgcccag
gtgtcaaggagtctcttgtctag
H.s. SCN5A
(SEQ ID NO: 10)
atggcaaacttcctattacctcggggcaccagcagcttccgcaggttca
cacgggagtccctggcagccatcgagaagcgcatggcagagaagcaagc
ccgcggctcaaccaccttgcaggagagccgagaggggctgcccgaggag
gaggctccccggccccagctggacctgcaggcctccaaaaagctgccag
atctctatggcaatccaccccaagagctcatcggagagcccctggagga
cctggaccccttctatagcacccaaaagactttcatcgtactgaataaa
ggcaagaccatcttccggttcagtgccaccaacgccttgtatgtcctca
gtcccttccaccccatccggagagcggctgtgaagattctggttcactc
gctcttcaacatgctcatcatgtgcaccatcctcaccaactgcgtgttc
atggcccagcacgaccctccaccctggaccaagtatgtcgagtacacct
tcaccgccatttacacctttgagtctctggtcaagattctggctcgagg
cttctgcctgcacgcgttcactttccttcgggacccatggaactggctg
gactttagtgtgattatcatggcatacacaactgaatttgtggacctgg
gcaatgtctcagccttacgcaccttccgagtcctccgggccctgaaaac
tatatcagtcatttcagggctgaagaccatcgtgggggccctgatccag
tctgtgaagaagctggctgatgtgatggtcctcacagtcttctgcctca
gcgtctttgccctcatcggcctgcagctcttcatgggcaacctaaggca
caagtgcgtgcgcaacttcacagcgctcaacggcaccaacggctccgtg
gaggccgacggcttggtctgggaatccctggacctttacctcagtgatc
cagaaaattacctgctcaagaacggcacctctgatgtgttactgtgtgg
gaacagctctgacgctgggacatgtccggagggctaccggtgcctaaag
gcaggcgagaaccccgaccacggctacaccagcttcgattcctttgcct
gggcctttcttgcactcttccgcctgatgacgcaggactgctgggagcg
cctctatcagcagaccctcaggtccgcagggaagatctacatgatcttc
ttcatgcttgtcatcttcctggggtccttctacctggtgaacctgatcc
tggccgtggtcgcaatggcctatgaggagcaaaaccaagccaccatcgc
tgagaccgaggagaaggaaaagcgcttccaggaggccatggaaatgctc
aagaaagaacacgaggccctcaccatcaggggtgtggataccgtgtccc
gtagctccttggagatgtcccctttggccccagtaaacagccatgagag
aagaagcaagaggagaaaacggatgtcttcaggaactgaggagtgtggg
gaggacaggctccccaagtctgactcagaagatggtcccagagcaatga
atcatctcagcctcacccgtggcctcagcaggacttctatgaagccacg
ttccagccgcgggagcattttcacctttcgcaggcgagacctgggttct
gaagcagattttgcagatgatgaaaacagcacagcgggggagagcgaga
gccaccacacatcactgctggtgccctggcccctgcgccggaccagtgc
ccagggacagcccagtcccggaacctcggctcctggccacgccctccat
ggcaaaaagaacagcactgtggactgcaatggggtggtctcattactgg
gggcaggcgacccagaggccacatccccaggaagccacctcctccgccc
tgtgatgctagagcacccgccagacacgaccacgccatcggaggagcca
ggcgggccccagatgctgacctcccaggctccgtgtgtagatggcttcg
aggagccaggagcacggcagcgggccctcagcgcagtcagcgtcctcac
cagcgcactggaagagttagaggagtctcgccacaagtgtccaccatgc
tggaaccgtctcgcccagcgctacctgatctgggagtgctgcccgctgt
ggatgtccatcaagcagggagtgaagttggtggtcatggacccgtttac
tgacctcaccatcactatgtgcatcgtactcaacacactcttcatggcg
ctggagcactacaacatgacaagtgaattcgaggagatgctgcaggtcg
gaaacctggtcttcacagggattttcacagcagagatgaccttcaagat
cattgccctcgacccctactactacttccaacagggctggaacatcttc
gacagcatcatcgtcatccttagcctcatggagctgggcctgtcccgca
tgagcaacttgtcggtgctgcgctccttccgcctgctgcgggtcttcaa
gctggccaaatcatggcccaccctgaacacactcatcaagatcatcggg
aactcagtgggggcactggggaacctgacactggtgctagccatcatcg
tgttcatctttgctgtggtgggcatgcagctctttggcaagaactactc
ggagctgagggacagcgactcaggcctgctgcctcgctggcacatgatg
gacttctttcatgccttcctcatcatcttccgcatcctctgtggagagt
ggatcgagaccatgtgggactgcatggaggtgtcggggcagtcattatg
cctgctggtcttcttgcttgttatggtcattggcaaccttgtggtcctg
aatctcttcctggccttgctgctcagctccttcagtgcagacaacctca
cagcccctgatgaggacagagagatgaacaacctccagctggccctggc
ccgcatccagaggggcctgcgctttgtcaagcggaccacctgggatttc
tgctgtggtctcctgcggcagcggcctcagaagcccgcagcccttgccg
cccagggccagctgcccagctgcattgccaccccctactccccgccacc
cccagagacggagaaggtgcctcccacccgcaaggaaacacggtttgag
gaaggcgagcaaccaggccagggcacccccggggatccagagcccgtgt
gtgtgcccatcgctgtggccgagtcagacacagatgaccaagaagaaga
tgaggagaacagcctgggcacggaggaggagtccagcaagcagcaggaa
tcccagcctgtgtccggtggcccagaggcccctccggattccaggacct
ggagccaggtgtcagcgactgcctcctctgaggccgaggccagtgcatc
tcaggccgactggcggcagcagtggaaagcggaaccccaggccccaggg
tgcggtgagaccccagaggacagttgctccgagggcagcacagcagaca
tgaccaacaccgctgagctcctggagcagatccctgacctcggccagga
tgtcaaggacccagaggactgcttcactgaaggctgtgtccggcgctgt
ccctgctgtgcggtggacaccacacaggccccagggaaggtctggtggc
ggttgcgcaagacctgctaccacatcgtggagcacagctggttcgagac
attcatcatcttcatgatcctactcagcagtggagcgctggccttcgag
gacatctacctagaggagcggaagaccatcaaggttctgcttgagtatg
ccgacaagatgttcacatatgtcttcgtgctggagatgctgctcaagtg
ggtggcctacggcttcaagaagtacttcaccaatgcctggtgctggctc
gacttcctcatcgtagacgtctctctggtcagcctggtggccaacaccc
tgggctttgccgagatgggccccatcaagtcactgcggacgctgcgtgc
actccgtcctctgagagctctgtcacgatttgagggcatgagggtggtg
gtcaatgccctggtgggcgccatcccgtccatcatgaacgtcctcctcg
tctgcctcatcttctggctcatcttcagcatcatgggcgtgaacctctt
tgcggggaagtttgggaggtgcatcaaccagacagagggagacttgcct
ttgaactacaccatcgtgaacaacaagagccagtgtgagtccttgaact
tgaccggagaattgtactggaccaaggtgaaagtcaactttgacaacgt
gggggccgggtacctggcccttctgcaggtggcaacatttaaaggctgg
atggacattatgtatgcagctgtggactccagggggtatgaagagcagc
ctcagtgggaatacaacctctacatgtacatctattttgtcattttcat
catctttgggtctttcttcaccctgaacctctttattggtgtcatcatt
gacaacttcaaccaacagaagaaaaagttagggggccaggacatcttca
tgacagaggagcagaagaagtactacaatgccatgaagaagctgggctc
caagaagccccagaagcccatcccacggcccctgaacaagtaccagggc
ttcatattcgacattgtgaccaagcaggcctttgacgtcaccatcatgt
ttctgatctgcttgaatatggtgaccatgatggtggagacagatgacca
aagtcctgagaaaatcaacatcttggccaagatcaacctgctctttgtg
gccatcttcacaggcgagtgtattgtcaagctggctgccctgcgccact
actacttcaccaacagctggaatatcttcgacttcgtggttgtcatcct
ctccatcgtgggcactgtgctctcggacatcatccagaagtacttcttc
tccccgacgctcttccgagtcatccgcctggcccgaataggccgcatcc
tcagactgatccgaggggccaaggggatccgcacgctgctctttgccct
catgatgtccctgcctgccctcttcaacatcgggctgctgctcttcctc
gtcatgttcatctactccatctttggcatggccaacttcgcttatgtca
agtgggaggctggcatcgacgacatgttcaacttccagaccttcgccaa
cagcatgctgtgcctcttccagatcaccacgtcggccggctgggatggc
ctcctcagccccatcctcaacactgggccgccctactgcgaccccactc
tgcccaacagcaatggctctcggggggactgcgggagcccagccgtggg
catcctcttcttcaccacctacatcatcatctccttcctcatcgtggtc
aacatgtacattgccatcatcctggagaacttcagcgtggccacggagg
agagcaccgagcccctgagtgaggacgacttcgatatgttctatgagat
ctgggagaaatttgacccagaggccactcagtttattgagtattcggtc
ctgtctgactttgccgatgccctgtctgagccactccgtatcgccaagc
ccaaccagataagcctcatcaacatggacctgcccatggtgagtgggga
ccgcatccattgcatggacattctctttgccttcaccaaaagggtcctg
ggggagtctggggagatggacgccctgaagatccagatggaggagaagt
tcatggcagccaacccatccaagatctcctacgagcccatcaccaccac
actccggcgcaagcacgaagaggtgtcggccatggttatccagagagcc
ttccgcaggcacctgctgcaacgctctttgaagcatgcctccttcctct
tccgtcagcaggcgggcagcggcctctccgaagaggatgcccctgagcg
agagggcctcatcgcctacgtgatgagtgagaacttctcccgacccctt
ggcccaccctccagctcctccatctcctccacttccttcccaccctcct
atgacagtgtcactagagccaccagcgataacctccaggtgcgggggtc
tgactacagccacagtgaagatctcgccgacttccccccttctccggac
agggaccgtgagtccatcgtgtga
H.s. SCN7A
(SEQ ID NO: 11)
atgttggcttcaccagaacctaagggccttgttcccttcactaaagagt
cttttgaacttataaaacagcatattgctaaaacacataatgaagacca
tgaagaagaagacttaaagccaactcctgatttggaagttggcaaaaag
cttccatttatttatggaaacctttctcaaggaatggtgtcagagccct
tggaagatgtggacccatattactacaagaaaaaaaatactttcatagt
attaaataaaaatagaacaatcttcagattcaatgcggcttccatcttg
tgtacattgtctcctttcaattgtattagaagaacaactatcaaggttt
tggtacatccctttttccaactgtttattctaattagtgtcctgattga
ttgcgtattcatgtccctgactaatttgccaaaatggagaccagtatta
gagaatactttgcttggaatttacacatttgaaatacttgtaaaactct
ttgcaagaggtgtctgggcaggatcattttccttcctcggtgatccatg
gaactggctcgatttcagcgtaactgtgtttgaggttattataagatac
tcacctctggacttcattccaacgcttcaaactgcaagaactttgagaa
ttttaaaaattattcctttaaatcaaggtctgaaatcccttgtaggggt
cctgatccactgcttgaagcagcttattggtgtcattatcctaactctg
ttttttctgagcatattttctctaattgggatggggctcttcatgggca
acttgaaacataaatgttttcgatggccccaagagaatgaaaatgaaac
cctgcacaacagaactggaaacccatattatattcgagaaacagaaaac
ttttattatttggaaggagaaagatatgctctcctttgtggcaacagga
cagatgctggtcagtgtcctgaaggatatgtgtgtgtaaaagctggcat
aaatcctgatcaaggcttcacaaattttgacagttttggctgggcctta
tttgccctatttcggttaatggctcaggattaccctgaagtactttatc
accagatactttatgcttctgggaaggtctacatgatattttttgtggt
ggtaagttttttgttttccttttatatggcaagtttgttcttaggcata
cttgccatggcctatgaagaagaaaagcagagagttggtgaaatatcta
agaagattgaaccaaaatttcaacagactggaaaagaacttcaagaagg
aaatgaaacagatgaggccaagaccatacaaatagaaatgaagaaaagg
tcaccaatttccacagacacatcattggatgtgttggaagatgctactc
tcagacataaggaagaacttgaaaaatccaagaagatatgcccattata
ctggtataagtttgctaaaactttcttgatctggaattgttctccctgt
tggttaaaattgaaagagtttgtccataggattataatggcaccattta
ctgatcttttccttatcatatgcataattttaaacgtatgttttctgac
cttggagcattatccaatgagtaaacaaactaacactcttctcaacatt
ggaaacctggttttcattggaattttcacagcagaaatgatttttaaaa
taattgcaatgcatccatatgggtatttccaagtaggttggaacatttt
tgatagcatgatagtgttccatggtttaatagaactttgtctagcaaat
gttgcaggaatggctcttcttcgattattcaggatgttaagaattttca
agttgggaaagtattggccaacattccagattttgatgtggtctcttag
taactcatgggtggccctgaaagacttggtcctgttgttgttcacattc
atcttcttttctgctgcattcggcatgaagctgtttggtaagaattatg
aagaatttgtctgccacatagacaaagactgtcaactcccacgctggca
catgcatgactttttccactccttcctgaatgtgttccgaattctctgt
ggagagtgggtagagaccttgtgggactgtatggaggttgcaggccaat
cctggtgtattcctttttacctgatggtcattttaattggaaatttact
ggtactttacctgtttctggcattggtgagctcatttagttcatgcaag
gatgtaacagctgaagagaataatgaagcaaaaaatctccagcttgcag
tggcaagaattaaaaaaggaataaactatgtgcttcttaaaatactatg
caaaacacaaaatgtcccaaaggacacaatggaccatgtaaatgaggta
tatgttaaagaagatatttctgaccataccctttctgaattgagcaaca
cccaagattttctcaaagataaggaaaaaagcagtggcacagagaaaaa
cgctactgaaaatgagagccaatcacttatccccagtcctagtgtctca
gaaactgtaccaattgcttcaggagaatctgatatagaaaatctggata
ataaggagattcagagtaagtctggtgatggaggcagcaaagagaaaat
aaagcaatctagctcatctgaatgcagtactgttgatattgctatctct
gaagaagaagaaatgttctatggaggtgaaagatcaaagcatctgaaaa
atggttgcagacgcggatcttcacttggtcaaatcagtggagcatccaa
gaaaggaaaaatctggcagaacatcaggaaaacctgctgcaagattgta
gagaacaattggtttaagtgttttattgggcttgttactctgctcagca
ctggcactctggcttttgaagatatatatatggatcagagaaagacaat
taaaattttattagaatatgctgacatgatctttacttatatcttcatt
ctggaaatgcttctaaaatggatggcatatggttttaaggcctatttct
ctaatggctggtacaggctggacttcgtggttgttattgtgttttgtct
tagcttaataggcaaaactcgggaagaactaaaacctcttatttccatg
aaattccttcggcccctcagagttctatctcaatttgaaagaatgaagg
tggttgtgagagctttgatcaaaacaaccttacccactttgaatgtgtt
tcttgtctgcctgatgatctggctgatttttagtatcatgggagtagac
ttatttgctggcagattctatgaatgcattgacccaacaagtggagaaa
ggtttccttcatctgaagtcatgaataagagtcggtgtgaaagccttct
gtttaacgaatccatgctatgggaaaatgcaaaaatgaactttgataat
gttggaaatggtttcctttctctgcttcaagtagcaacatttaatggat
ggatcactattatgaattcagcaattgattctgttgctgttaatataca
gcctcattttgaagtcaacatctacatgtattgttactttatcaacttt
attatatttggagtatttctccctctgagtatgctgattactgttatta
ttgataatttcaacaagcataaaataaagctgggaggctcaaatatctt
tataacggttaaacagagaaaacagtaccgcaggctgaagaagctaatg
tatgaggattctcaaagaccagtacctcgcccattaaacaagctccaag
gattcatctttgatgtggtaacaagccaagcttttaatgtcattgttat
ggttcttatatgtttccaagcaatagccatgatgatagacactgatgtt
cagagtctacaaatgtccattgctctctactggattaactcaatttttg
ttatgctatatactatggaatgtatactgaagctcatcgctttccgttg
tttttatttcaccattgcgtggaacatttttgattttatggtggttatt
ttctccatcacaggactatgtctgcctatgacagtaggatcctaccttg
tgcctccttcacttgtgcaactgatacttctctcacggatcattcacat
gctgcgtcttggaaaaggaccaaaggtgtttcataatctgatgcttcct
ttgatgctgtccctcccagcattattgaacatcattcttctcatcttcc
tggtcatgttcatctatgccgtatttggaatgtataattttgcctatgt
taaaaaagaagctggaattaatgatgtgtctaattttgaaacctttggc
aacagtatgctctgtctttttcaagttgcaatatttgctggttgggatg
ggatgcttgatgcaattttcaacagtaaatggtctgactgtgatcctga
taaaattaaccctgggactcaagttagaggagattgtgggaacccctct
gttgggattttttattttgtcagttatatcctcatatcatggctgatca
ttgtaaatatgtacattgttgttgtcatggagtttttaaatattgcttc
taagaagaaaaacaagaccttgagtgaagatgattttaggaaattcttt
caggtatggaaaaggtttgatcctgataggacccagtacatagactcta
gcaagctttcagattttgcagctgctcttgatcctcctcttttcatggc
aaaaccaaacaagggccagctcattgctttggacctccccatggctgtt
ggggacagaattcattgcctcgatatcttacttgcttttacaaagagag
ttatgggtcaagatgtgaggatggagaaagttgtttcagaaatagaatc
agggtttttgttagccaacccttttaagatcacatgtgagccaattacg
actactttgaaacgaaaacaagaggcagtttcagcaaccatcattcaac
gtgcttataaaaattaccgcttgaggcgaaatgacaaaaatacatcaga
tattcatatgatagatggtgacagagatgttcatgctactaaagaaggt
gcctattttgacaaagctaaggaaaagtcacctattcaaagccagatct
aa
H.s. SCN8A
(SEQ ID NO: 12)
atggcagcgcggctgcttgcaccaccaggccctgatagtttcaagcctt
tcacccctgagtcactggcaaacattgagaggcgcattgctgagagcaa
gctcaagaaaccaccaaaggccgatggcagtcatcgggaggacgatgag
gacagcaagcccaagccaaacagcgacctggaagcagggaagagtttgc
ctttcatctacggggacatcccccaaggcctggttgcagttcccctgga
ggactttgacccatactatttgacgcagaaaacctttgtagtattaaac
agagggaaaactctcttcagatttagtgccacgcctgccttgtacattt
taagtccttttaacctgataagaagaatagctattaaaattttgataca
ttcagtatttagcatgatcattatgtgcactattttgaccaactgtgta
ttcatgacttttagtaaccctcctgactggtcgaagaatgtggagtaca
cgttcacagggatttatacatttgaatcactagtgaaaatcattgcaag
aggtttctgcatagatggctttacctttttacgggacccatggaactgg
ttagatttcagtgtcatcatgatggcgtatataacagagtttgtaaacc
taggcaatgtttcagctctacgcactttcagggtactgagggctttgaa
aactatttcggtaatcccaggcctgaagacaattgtgggtgccctgatt
cagtctgtgaagaaactgtcagatgtgatgatcctgacagtgttctgcc
tgagtgtttttgccttgatcggactgcagctgttcatggggaaccttcg
aaacaagtgtgttgtgtggcccataaacttcaacgagagctatcttgaa
aatggcaccaaaggctttgattgggaagagtatatcaacaataaaacaa
atttctacacagttcctggcatgctggaacctttactctgtgggaacag
ttctgatgctgggcaatgcccagagggataccagtgtatgaaagcagga
aggaaccccaactatggttacacaagttttgacacttttagctgggcct
tcttggcattatttcgccttatgacccaggactattgggaaaacttgta
tcaattgactttacgagcagccgggaaaacatacatgatcttcttcgtc
ttggtcatctttgtgggttctttctatctggtgaacttgatcttggctg
tggtggccatggcttatgaagaacagaatcaggcaacactggaggaggc
agaacaaaaagaggctgaatttaaagcaatgttggagcaacttaagaag
caacaggaagaggcacaggctgctgcgatggccacttcagcaggaactg
tctcagaagatgccatagaggaagaaggtgaagaaggagggggctcccc
tcggagctcttctgaaatctctaaactcagctcaaagagtgcaaaggaa
agacgtaacaggagaaagaagaggaagcaaaaggaactctctgaaggag
aggagaaaggggatcccgagaaggtgtttaagtcagagtcagaagatgg
catgagaaggaaggcctttcggctgccagacaacagaatagggaggaaa
ttttccatcatgaatcagtcactgctcagcatcccaggctcgcccttcc
tctcccgccacaacagcaagagcagcatcttcagtttcaggggacctgg
gcggttccgagacccgggctccgagaatgagttcgcggatgacgagcac
agcacggtggaggagagcgagggccgccgggactccctcttcatcccca
tccgggcccgcgagcgccggagcagctacagcggctacagcggctacag
ccagggcagccgctcctcgcgcatcttccccagcctgcggcgcagcgtg
aagcgcaacagcacggtggactgcaacggcgtggtgtccctcatcggcg
gccccggctcccacatcggcgggcgtctcctgccagaggctacaactga
ggtggaaattaagaagaaaggccctggatctcttttagtttccatggac
caattagcctcctacgggcggaaggacagaatcaacagtataatgagtg
ttgttacaaatacactagtagaagaactggaagagtctcagagaaagtg
cccgccatgctggtataaatttgccaacactttcctcatctgggagtgc
cacccctactggataaaactgaaagagattgtgaacttgatagttatgg
acccttttgtggatttagccatcaccatctgcatcgtcctgaatacact
gtttatggcaatggagcaccatcctatgacaccacaatttgaacatgtc
ttggctgtaggaaatctggttttcactggaattttcacagcggaaatgt
tcctgaagctcatagccatggatccctactattatttccaagaaggttg
gaacatttttgacggatttattgtctccctcagtttaatggaactgagt
ctagcagacgtggaggggctttcagtgctgcgatctttccgattgctcc
gagtcttcaaattggccaaatcctggcccaccctgaacatgctaatcaa
gatatggaaattcagtgggtgccctgggcaacctgacactggtgctggc
cattattgtcttcatctttgccgtggtggggatgcaactctttggaaaa
agctacaaagagtgtgtctgcaagatcaaccaggactgtgaactccctc
gctggcatatgcatgactttttccattccttcctcattgtctttcgagt
gttgtgcggggagtggattgagaccatgtgggactgcatggaagtggca
ggccaggccatgtgcctcattgtctttatgatggtcatggtgattggca
acttggtggtgctgaacctgtttctggccttgctcctgagctccttcag
tgcagacaacctggctgccacagatgacgatggggaaatgaacaacctc
cagatctcagtgatccgtatcaagaagggtgtggcctggaccaaactaa
aggtgcacgccttcatgcaggcccactttaagcagcgtgaggctgatga
ggtgaagcctctggatgagttgtatgaaaagaaggccaactgtatcgcc
aatcacaccggtgcagacatccaccggaatggtgacttccagaagaatg
gcaatggcacaaccagcggcattggcagcagcgtggagaagtacatcat
tgatgaggaccacatgtccttcatcaacaaccccaacttgactgtacgg
gtacccattgctgtgggcgagtctgactttgagaacctcaacacagagg
atgttagcagcgagtcggatcctgaaggcagcaaagataaactagatga
caccagctcctctgaaggaagcaccattgatatcaaaccagaagtagaa
gaggtccctgtggaacagcctgaggaatacttggatccagatgcctgct
tcacagaaggttgtgtccagcggttcaagtgctgccaggtcaacatcga
ggaagggctaggcaagtcttggtggatcctgcggaaaacctgcttcctc
atcgtggagcacaactggtttgagaccttcatcatcttcatgattctgc
tgagcagtggcgccctggccttcgaggacatctacattgagcagagaaa
gaccatccgcaccatcctggaatatgctgacaaagtcttcacctatatc
ttcatcctggagatgttgctcaagtggacagcctatggcttcgtcaagt
tcttcaccaatgcctggtgttggctggacttcctcattgtggctgtctc
tttagtcagccttatagctaatgccctgggctactcggaactaggtgcc
ataaagtcccttaggaccctaagagctttgagacccttaagagccttat
cacgatttgaagggatgagggtggtggtgaatgccttggtgggcgccat
cccctccatcatgaatgtgctgctggtgtgtctcatcttctggctgatt
ttcagcatcatgggagttaacttgtttgcgggaaagtaccactactgct
ttaatgagacttctgaaatccgatttgaaattgaagatgtcaacaataa
aactgaatgtgaaaagcttatggaggggaacaatacagagatcagatgg
aagaacgtgaagatcaactttgacaatgttggggcaggatacctggccc
ttcttcaagtagcaaccttcaaaggctggatggacatcatgtatgcagc
tgtagattcccggaagcctgatgagcagcctaagtatgaggacaatatc
tacatgtacatctattttgtcatcttcatcatcttcggctccttcttca
ccctgaacctgttcattggtgtcatcattgataacttcaatcaacaaaa
gaaaaagttcggaggtcaggacatcttcatgaccgaagaacagaagaag
tactacaatgccatgaaaaagctgggctcaaagaagccacagaaaccta
ttccccgccccttgaacaaaatccaaggaatcgtctttgattttgtcac
tcagcaagcctttgacattgttatcatgatgctcatctgccttaacatg
gtgacaatgatggtggagacagacactcaaagcaagcagatggagaaca
tcctctactggattaacctggtgtttgttatcttcttcacctgtgagtg
tgggctcaaaatgtttgcgttgaggcactactacttcaccattggctgg
aacatcttcgacttcgtggtagtcatcctctccattgtgggaatgttcc
tggcagatataattgagaaatactttgtttccccaaccctattccgagt
catccgattggcccgtattgggcgcatcttgcgtctgatcaaaggcgcc
aaagggattcgtaccctgctctttgccttaatgatgtccttgcctgccc
tgttcaacatcggccttctgctcttcctggtcatgttcatcttctccat
ttttgggatgtccaattttgcatatgtgaagcacgaggctggtatcgat
gacatgttcaactttgagacatttggcaacagcatgatctgcctgtttc
aaatcacaacctcagctggttgggatggcctgctgctgcccatcctaaa
ccgcccccctgactgcagcctagataaggaacacccagggagtggcttt
aagggagattgtgggaacccctcagtgggcatcttcttctttgtaagct
acatcatcatctctttcctaattgtcgtgaacatgtacattgccatcat
cctggagaacttcagtgtagccacagaggaaagtgcagaccctctgagt
gaggatgactttgagaccttctatgagatctgggagaagttcgaccccg
atgccacccagttcattgagtactgtaagctggcagactttgcagatgc
cttggagcatcctctccgagtgcccaagcccaataccattgagctcatc
gctatggatctgccaatggtgagcggggatcgcatccactgcttggaca
tcctttttgccttcaccaagcgggtcctgggagatagcggggagttgga
catcctgcggcagcagatggaagagcggttcgtggcatccaatccttcc
aaagtgtcttacgagccaatcacaaccacactgcgtcgcaagcaggagg
aggtatctgcagtggtcctgcagcgtgcctaccggggacatttggcaag
gcggggcttcatctgcaaaaagacaacttctaataagctggagaatgga
ggcacacaccgggagaaaaaagagagcaccccatctacagcctccctcc
cgtcctatgacagtgtaactaaacctgaaaaggagaaacagcagcgggc
agaggaaggaagaagggaaagagccaaaagacaaaaagaggtcagagaa
tccaagtgttag
H.s. SCN9A
(SEQ ID NO: 13)
atggcaatgttgcctcccccaggacctcagagctttgtccatttcacaa
aacagtctcttgccctcattgaacaacgcattgctgaaagaaaatcaaa
ggaacccaaagaagaaaagaaagatgatgatgaagaagccccaaagcca
agcagtgacttggaagctggcaaacaactgcccttcatctatggggaca
ttcctcccggcatggtgtcagagcccctggaggacttggacccctacta
tgcagacaaaaagactttcatagtattgaacaaagggaaaacaatcttc
cgtttcaatgccacacctgctttatatatgctttctccttcagtcctct
aagaagaatatctattaagatttagtacactcctattcagcatgctcat
catgtgcactatctgacaaactgcatatttatgaccatgaataacccgc
cggactggaccaaaaatgtcgagtacacttttactggaatatatacttt
tgaatcacttgtaaaaatccttgcaagaggcttctgtgtaggagaattc
acttttcttcgtgacccgtggaactggctggattttgtcgtcattgttt
ttgcgtatttaacagaatttgtaaacctaggcaatgtttcagctcttcg
aactttcagagtattgagagctttgaaaactatttctgtaatcccaggc
ctgaagacaattgtaggggctttgatccagtcagtgaagaagctttctg
atgtcatgatcctgactgtgttctgtctgagtgtgtttgcactaattgg
actacagctgttcatgggaaacctgaagcataaatgttttcgaaattca
cttgaaaataatgaaacattagaaagcataatgaataccctagagagtg
aagaagactttagaaaatatttttattacttggaaggatccaaagatgc
tctcctttgtggtttcagcacagattcaggtcagtgtccagaggggtac
acctgtgtgaaaattggcagaaaccctgattatggctacacgagctttg
acactttcagctgggccttcttagccttgtttaggctaatgacccaaga
ttactgggaaaacctttaccaacagacgctgcgtgctgctggcaaaacc
tacatgatcttctttgtcgtagtgattttcctgggctccttttatctaa
taaacttgatcctggctgtggttgccatggcatatgaagaacagaacca
ggcaaacattgaagaagctaaacagaaagaattagaacttcaacagatg
ttagaccgtcttaaaaaagagcaagaagaagctgaggcaattgcagcgg
cagcggctgaatatacaagtattaggagaagcagaattatgggcctctc
agagagttcttctgaaacatccaaactgagctctaaaagtgctaaagaa
agaagaaacagaagaaagaaaaagaatcaaaagaagctctccagtggag
aggaaaagggagatgctgagaaattgtcgaaatcagaatcagaggacag
catcagaagaaaaagtttccaccttggtgtcgaagggcataggcgagca
catgaaaagaggttgtctacccccaatcagtcaccactcagcattcgtg
gctccttgttttctgcaaggcgaagcagcagaacaagtctttttagttt
caaaggcagaggaagagatataggatctgagactgaatttgccgatgat
gagcacagcatttttggagacaatgagagcagaaggggctcactgtttg
tgccccacagaccccaggagcgacgcagcagtaacatcagccaagccag
taggtccccaccaatgctgccggtgaacgggaaaatgcacagtgctgtg
gactgcaacggtgtggtctccctggttgatggacgctcagccctcatgc
tccccaatggacagcttctgccagagggcacgaccaatcaaatacacaa
gaaaaggcgttgtagttcctatctcctttcagaggatatgctgaatgat
cccaacctcagacagagagcaatgagtagagcaagcatattaacaaaca
ctgtggaagaacttgaagagtccagacaaaaatgtccaccttggtggta
cagatttgcacacaaattcttgatctggaattgctctccatattggata
aaattcaaaaagtgtatctattttattgtaatggatccttttgtagatc
ttgcaattaccatttgcatagttttaaacacattatttatggctatgga
acaccacccaatgactgaggaattcaaaaatgtacttgctataggaaat
ttggtctttactggaatctttgcagctgaaatggtattaaaactgattg
ccatggatccatatgagtatttccaagtaggctggaatatttttgacag
ccttattgtgactttaagtttagtggagctctttctagcagatgtggaa
ggattgtcagttctgcgatcattcagactgctccgagtcttcaagttgg
caaaatcctggccaacattgaacatgctgattaagatcattggtaactc
agtaggggctctaggtaacctcaccttagtgttggccatcatcgtcttc
atttttgctgtggtcggcatgcagctctttggtaagagctacaaagaat
gtgtctgcaagatcaatgatgactgtacgctcccacggtggcacatgaa
cgacttcttccactccttcctgattgtgttccgcgtgctgtgtggagag
tggatagagaccatgtgggactgtatggaggtcgctggtcaagctatgt
gccttattgtttacatgatggtcatggtcattggaaacctggtggtcct
aaacctatttctggccttattattgagctcatttagttcagacaatctt
acagcaattgaagaagaccctgatgcaaacaacctccagattgcagtga
ctagaattaaaaagggaataaattatgtgaaacaaaccttacgtgaatt
tattctaaaagcattttccaaaaagccaaagatttccagggagataaga
caagcagaagatctgaatactaagaaggaaaactatatttctaaccata
cacttgctgaaatgagcaaaggtcacaatttcctcaaggaaaaagataa
aatcagtggttttggaagcagcgtggacaaacacttgatggaagacagt
gatggtcaatcatttattcacaatcccagcctcacagtgacagtgccaa
ttgcacctggggaatccgatttggaaaatatgaatgctgaggaacttag
cagtgattcggatagtgaatacagcaaagtgagattaaaccggtcaagc
tcctcagagtgcagcacagttgataaccctttgcctggagaaggagaag
aagcagaggctgaacctatgaattccgatgagccagaggcctgtttcac
agatggttgtgtacggaggttctcatgctgccaagttaacatagagtca
gggaaaggaaaaatctggtggaacatcaggaaaacctgctacaagattg
ttgaacacagttggtttgaaagcttcattgtcctcatgatcctgctcag
cagtggtgccctggcttttgaagatatttatattgaaaggaaaaagacc
attaagattatcctggagtatgcagacaagatcttcacttacatcttca
ttctggaaatgcttctaaaatggatagcatatggttataaaacatattt
caccaatgcctggtgttggctggatttcctaattgttgatgtttctttg
gttactttagtggcaaacactcttggctactcagatcttggccccatta
aatcccttcggacactgagagctttaagacctctaagagccttatctag
atttgaaggaatgagggtcgttgtgaatgcactcataggagcaattcct
tccatcatgaatgtgctacttgtgtgtcttatattctggctgatattca
gcatcatgggagtaaatttgtttgctggcaagttctatgagtgtattaa
caccacagatgggtcacggtttcctgcaagtcaagttccaaatcgttcc
gaatgttttgcccttatgaatgttagtcaaaatgtgcgatggaaaaacc
tgaaagtgaactttgataatgtcggacttggttacctatctctgcttca
agttgcaacttttaagggatggacgattattatgtatgcagcagtggat
tctgttaatgtagacaagcagcccaaatatgaatatagcctctacatgt
atatttattttgtcgtctttatcatctttgggtcattcttcactttgaa
cttgttcattggtgtcatcatagataatttcaaccaacagaaaaagaag
cttggaggtcaagacatctttatgacagaagaacagaagaaatactata
atgcaatgaaaaagctggggtccaagaagccacaaaagccaattcctcg
accagggaacaaaatccaaggatgtatatttgacctagtgacaaatcaa
gcctttgatattagtatcatggttcttatctgtctcaacatggtaacca
tgatggtagaaaaggagggtcaaagtcaacatatgactgaagttttata
ttggataaatgtggtttttataatccttttcactggagaatgtgtgcta
aaactgatctccctcagacactactacttcactgtaggatggaatattt
ttgattttgtggttgtgattatctccattgtaggtatgtttctagctga
tttgattgaaacgtattttgtgtcccctaccctgttccgagtgatccgt
cttgccaggattggccgaatcctacgtctagtcaaaggagcaaagggga
tccgcacgctgctctttgctttgatgatgtcccttcctgcgttgtttaa
catcggcctcctgctcttcctggtcatgttcatctacgccatctttgga
atgtccaactttgcctatgttaaaaaggaagatggaattaatgacatgt
tcaattttgagacctttggcaacagtatgatttgcctgttccaaattac
aacctctgctggctgggatggattgctagcacctattcttaacagtaag
ccacccgactgtgacccaaaaaaagttcatcctggaagttcagttgaag
gagactgtggtaacccatctgttggaatattctactttgttagttatat
catcatatccttcctggttgtggtgaacatgtacattgcagtcatactg
gagaattttagtgttgccactgaagaaagtactgaacctctgagtgagg
atgactttgagatgttctatgaggtttgggagaagtttgatcccgatgc
gacccagtttatagagttctctaaactctctgattttgcagctgccctg
gatcctcctcttctcatagcaaaacccaacaaagtccagctcattgcca
tggatctgcccatggttagtggtgaccggatccattgtcttgacatctt
atttgcttttacaaagcgtgttttgggtgagagtggggagatggattct
cttcgttcacagatggaagaaaggttcatgtctgcaaatccttccaaag
tgtcctatgaacccatcacaaccacactaaaacggaaacaagaggatgt
gtctgctactgtcattcagcgtgcttatagacgttaccgcttaaggcaa
aatgtcaaaaatatatcaagtatatacataaaagatggagacagagatg
atgatttactcaataaaaaagatatggcttttgataatgttaatgagaa
ctcaagtccagaaaaaacagatgccacttcatccaccacctctccacct
tcatatgatagtgtaacaaagccagacaaagagaaatatgaacaagaca
gaacagaaaaggaagacaaagggaaagacagcaaggaaagcaaaaaata
g
H.s. SCN10A
(SEQ ID NO: 14)
atggaattccccattggatccctcgaaactaacaacttccgtcgcttta
ctccggagtcactggtggagatagagaagcaaattgctgccaagcaggg
aacaaagaaagccagagagaagcatagggagcagaaggaccaagaagag
aagcctcggccccagctggacttgaaagcctgcaaccagctgcccaagt
tctatggtgagctcccagcagaactgatcggggagcccctggaggatct
agatccgttctacagcacacaccggacatttatggtgctgaacaaaggg
aggaccatttcccggtttagtgccactcgggccctgtggctattcagtc
ctttcaacctgatcagaagaacggccatcaaagtgtctgtccactcgtg
gttcagtttatttattacggtcactattttggttaattgtgtgtgcatg
acccgaactgaccttccagagaaaattgaatatgtcttcactgtcattt
acacctttgaagccttgataaagatactggcaagaggattttgtctaaa
tgagttcacgtacctgagagatccttggaactggctggattttagcgtc
attaccctggcatatgttggcacagcaatagatctccgtgggatctcag
gcctgcggacattcagagttcttagagcattaaaaacagtttctgtgat
cccaggcctgaaggtcattgtgggggccctgattcactcagtgaagaaa
ctggctgatgtgaccatcctcaccatcttctgcctaagtgtttttgcct
tggtggggctgcaactcttcaagggcaacctcaaaaataaatgtgtcaa
gaatgacatggctgtcaatgagacaaccaactactcatctcacagaaaa
ccagatatctacataaataagcgaggcacttctgaccccttactgtgtg
gcaatggatctgactcaggccactgccctgatggttatatctgccttaa
aacttctgacaacccggattttaactacaccagctttgattcctttgct
tgggctttcctctcactgttccgcctcatgacacaggattcctgggaac
gcctctaccagcagaccctgaggacttctgggaaaatctatatgatctt
ttttgtgctcgtaatcttcctgggatctttctacctggtcaacttgatc
ttggctgtagtcaccatggcgtatgaggagcagaaccaggcaaccactg
atgaaattgaagcaaaggagaagaagttccaggaggccctcgagatgct
ccggaaggagcaggaggtgctagcagcactagggattgacacaacctct
ctccactcccacaatggatcacctttaacctccaaaaatgccagtgaga
gaaggcatagaataaagccaagagtgtcagagggctccacagaagacaa
caaatcaccccgctctgatccttacaaccagcgcaggatgtcttttcta
ggcctcgcctctggaaaacgccgggctagtcatggcagtgtgttccatt
tccggtcccctggccgagatatctcactccctgagggagtcacagatga
tggagtctttcctggagaccacgaaagccatcggggctctctgctgctg
ggtgggggtgctggccagcaaggccccctccctagaagccctcttcctc
aacccagcaaccctgactccaggcatggagaagatgaacaccaaccgcc
gcccactagtgagcttgcccctggagctgtcgatgtctcggcattcgat
gcaggacaaaagaagactttcttgtcagcagaatacttagatgaacctt
tccgggcccaaagggcaatgagtgttgtcagtatcataacctccgtcct
tgaggaactcgaggagtctgaacagaagtgcccaccctgcttgaccagc
ttgtctcagaagtatctgatctgggattgctgccccatgtgggtgaagc
tcaagacaattctctttgggcttgtgacggatccctttgcagagctcac
catcaccttgtgcatcgtggtgaacaccatcttcatggccatggagcac
catggcatgagccctaccttcgaagccatgctccagataggcaacatcg
tctttaccatattttttactgctgaaatggtcttcaaaatcattgcctt
cgacccatactattatttccagaagaagtggaatatctttgactgcatc
atcgtcactgtgagtctgctagagctgggcgtggccaagaagggaagcc
tgtctgtgctgcggagcttccgcttgctgcgcgtattcaagctggccaa
atcctggcccaccttaaacacactcatcaagatcatcggaaactcagtg
ggggcactggggaacctcaccatcatcctggccatcattgtctttgtct
ttgctctggttggcaagcagctcctaggggaaaactaccgtaacaaccg
aaaaaatatctccgcgccccatgaagactggccccgctggcacatgcac
gacttcttccactctttcctcattgtcttccgtatcctctgtggagagt
ggattgagaacatgtgggcctgcatggaagttggccaaaaatccatatg
cctcatccttttcttgacggtgatggtgctagggaacctggtggtgctt
aacctgttcatcgccctgctattgaactctttcagtgctgacaacctca
cagccccggaggacgatggggaggtgaacaacctgcaggtggccctggc
acggatccaggtctttggccatcgtaccaaacaggctctttgcagcttc
ttcagcaggtcctgcccattcccccagcccaaggcagagcctgagctgg
tggtgaaactcccactctccagctccaaggctgagaaccacattgctgc
caacactgccagggggagctctggagggctccaagctcccagaggcccc
agggatgagcacagtgacttcatcgctaatccgactgtgtgggtctctg
tgcccattgctgagggtgaatctgatcttgatgacttggaggatgatgg
tggggaagatgctcagagcttccagcaggaagtgatccccaaaggacag
caggagcagctgcagcaagtcgagaggtgtggggaccacctgacaccca
ggagcccaggcactggaacatcttctgaggacctggctccatccctggg
tgagacgtggaaagatgagtctgttcctcaggtccctgctgagggagtg
gacgacacaagctcctctgagggcagcacggtggactgcctagatcctg
aggaaatcctgaggaagatccctgagctggcagatgacctggaagaacc
agatgactgcttcacagaaggatgcattcgccactgtccctgctgcaaa
ctggataccaccaagagtccatgggatgtgggctggcaggtgcgcaaga
cttgctaccgtatcgtggagcacagctggtttgagagcttcatcatctt
catgatcctgctcagcagtggatctctggcctttgaagactattacctg
gaccagaagcccacggtgaaagctttgctggagtacactgacagggtct
tcacctttatctttgtgttcgagatgctgcttaagtgggtggcctatgg
cttcaaaaagtacttcaccaatgcctggtgctggctggacttcctcatt
gtgaatatctcactgataagtctcacagcgaagattctggaatattctg
aagtggctcccatcaaagcccttcgaacccttcgcgctctgcggccact
gcgggctctttctcgatttgaaggcatgcgggtggtggtggatgccctg
gtgggcgccatcccatccatcatgaatgtcctcctcgtctgcctcatct
tctggctcatcttcagcatcatgggtgtgaacctcttcgcagggaagtt
ttggaggtgcatcaactataccgatggagagttttcccttgtacctttg
tcgattgtgaataacaagtctgactgcaagattcaaaactccactggca
gcttcttctgggtcaatgtgaaagtcaactttgataatgttgcaatggg
ttaccttgcacttctgcaggtggcaacctttaaaggctggatggacatt
atgtatgcagctgttgattcccgggaggtcaacatgcaacccaagtggg
aggacaacgtgtacatgtatttgtactttgtcatcttcatcatttttgg
aggcttcttcacactgaatctctttgttggggtcataattgacaacttc
aatcaacagaaaaaaaagttagggggccaggacatcttcatgacagagg
agcagaagaaatactacaatgccatgaagaagttgggctccaagaagcc
ccagaagcccatcccacggcccctgaacaagttccagggttttgtcttt
gacatcgtgaccagacaagcttttgacatcaccatcatggtcctcatct
gcctcaacatgatcaccatgatggtggagactgatgaccaaagtgaaga
aaagacgaaaattctgggcaaaatcaaccagttctttgtggccgtcttc
acaggcgaatgtgtcatgaagatgttcgctttgaggcagtactacttca
caaatggctggaatgtgtttgacttcattgtggtggttctctccattgc
gagcctgattttttctgcaattcttaagtcacttcaaagttacttctcc
ccaacgctcttcagagtcatccgcctggcccgaattggccgcatcctca
gactgatccgagcggccaaggggatccgcacactgctctttgccctcat
gatgtccctgcctgccctcttcaacatcgggctgttgctattccttgtc
atgttcatctactctatcttcggtatgtccagctttccccatgtgaggt
gggaggctggcatcgacgacatgttcaacttccagaccttcgccaacag
catgctgtgcctcttccagattaccacgtcggccggctgggatggcctc
ctcagccccatcctcaacacagggcccccctactgtgaccccaatctgc
ccaacagcaatggcaccagaggggactgtgggagcccagccgtaggcat
catcttcttcaccacctacatcatcatctccttcctcatcatggtcaac
atgtacattgcagtgattctggagaacttcaatgtggccacggaggaga
gcactgagcccctgagtgaggacgactttgacatgttctatgagacctg
ggagaagtttgacccagaggccactcagtttattaccttttctgctctc
tcggactttgcagacactctctctggtcccctgagaatcccaaaaccca
atcgaaatatactgatccagatggacctgcctttggtccctggagataa
gatccactgcttggacatcctttttgctttcaccaagaatgtcctagga
gaatccggggagttggattctctgaaggcaaatatggaggagaagttta
tggcaactaatctttcaaaatcatcctatgaaccaatagcaaccactct
ccgatggaagcaagaagacatttcagccactgtcattcaaaaggcctat
cggagctatgtgctgcaccgctccatggcactctctaacaccccatgtg
tgcccagagctgaggaggaggctgcatcactcccagatgaaggttttgt
tgcattcacagcaaatgaaaattgtgtactcccagacaaatctgaaact
gcttctgccacatcattcccaccgtcctatgagagtgtcactagaggcc
ttagtgatagagtcaacatgaggacatctagctcaatacaaaatgaaga
tgaagccaccagtatggagctgattgcccctgggccctag
H.s. SCN11A
(SEQ ID NO: 15)
atggatgacagatgctacccagtaatctttccagatgagcggaatttcc
gccccttcacttccgactctctggctgcaattgagaagcggattgccat
ccaaaaggagaaaaagaagtctaaagaccagacaggagaagtaccccag
cctcggcctcagcttgacctaaaggcctccaggaagttgcccaagctct
atggcgacattcctcgtgagctcataggaaagcctctggaagacttgga
cccattctaccgaaatcataagacatttatggtgttaaacagaaagagg
acaatctaccgcttcagtgccaagcatgccttgttcatttttgggcctt
tcaattcaatcagaagtttagccattagagtctcagtccattcattgtt
cagcatgttcattatcggcaccgttatcatcaactgcgtgttcatggct
acagggcctgctaaaaacagcaacagtaacaatactgacattgcagagt
gtgtcttcactgggatttatatttttgaagctttgattaaaatattggc
aagaggtttcattctggatgagttttctttccttcgagatccatggaac
tggctggactccattgtcattggaatagcgattgtgtcatatattccag
gaatcaccatcaaactattgcccctgcgtaccttccgtgtgttcagagc
tttgaaagcaatttcagtagtttcacgtctgaaggtcatcgtgggggcc
ttgctacgctctgtgaagaagctggtcaacgtgattatcctcaccttct
tttgcctcagcatctttgccctggtaggtcagcagctcttcatgggaag
tctgaacctgaaatgcatctcgagggactgtaaaaatatcagtaacccg
gaagcttatgaccattgctttgaaaagaaagaaaattcacctgaattca
aaatgtgtggcatctggatgggtaacagtgcctgttccatacaatatga
atgtaagcacaccaaaattaatcctgactataattatacgaattttgac
aactttggctggtcttttcttgccatgttccggctgatgacccaagatt
cctgggagaagctttatcaacagaccctgcgtactactgggctctactc
agtcttcttcttcattgtggtcattttcctgggctccttctacctgatt
aacttaaccctggctgttgttaccatggcatatgaggagcagaacaaga
atgtagctgcagagatagaggccaaggaaaagatgtttcaggaagccca
gcagctgttaaaggaggaaaaggaggctctggttgccatgggaattgac
agaagttcacttacttcccttgaaacatcatattttaccccaaaaaaga
gaaagctctttggtaataagaaaaggaagtccttctttttgagagagtc
tgggaaagaccagcctcctgggtcagattctgatgaagattgccaaaaa
aagccacagctcctagagcaaaccaaacgactgtcccagaatctatcac
tggaccactttgatgagcatggagatcctctccaaaggcagagagcact
gagtgctgtcagcatcctcaccatcaccatgaaggaacaagaaaaatca
caagagccttgtctcccttgtggagaaaacctggcatccaagtacctcg
tgtggaactgttgcccccagtggctgtgcgttaagaaggtcctgagaac
tgtgatgactgacccgtttactgagctggccatcaccatctgcatcatc
atcaacactgtcttcttggccatggagcatcacaagatggaggccagtt
ttgagaagatgttgaatatagggaatttggttttcactagcatttttat
agcagaaatgtgcctaaaaatcattgcgctcgatccctaccactacttt
cgccgaggctggaacatttttgacagcattgttgctcttctgagttttg
cagatgtaatgaactgtgtacttcaaaagagaagctggccattcttgcg
ttccttcagagtgctcagggtcttcaagttagccaaatcctggccaact
ttgaacacactaattaagataatcggcaactctgtcggagcccttggaa
gcctgactgtggtcctggtcattgtgatctttattttctcagtagttgg
catgcagctttttggccgtagcttcaattcccaaaagagtccaaaactc
tgtaacccgacaggcccgacagtctcatgtttacggcactggcacatgg
gggatttctggcactccttcctagtggtattccgcatcctctgcgggga
atggatcgaaaatatgtgggaatgtatgcaagaagcgaatgcatcatca
tcattgtgtgttattgtcttcatattgatcacggtgataggaaaacttg
tggtgctcaacctcttcattgccttactgctcaattcctttagcaatga
ggaaagaaatggaaacttagaaggagaggccaggaaaactaaagtccag
ttagcactggatcgattccgccgggctttttgttttgtgagacacactc
ttgagcatttctgtcacaagtggtgcaggaagcaaaacttaccacagca
aaaagaggtggcaggaggctgtgctgcacaaagcaaagacatcattccc
ctggtcatggagatgaaaaggggctcagagacccaggaggagcttggta
tactaacctctgtaccaaagaccctgggcgtcaggcatgattggacttg
gttggcaccacttgcggaggaggaagatgacgttgaattttctggtgaa
gataatgcacagcgcatcacacaacctgagcctgaacaacaggcctatg
agctccatcaggagaacaagaagcccacgagccagagagttcaaagtgt
ggaaattgacatgttctctgaagatgagcctcatctgaccatacaggat
ccccgaaagaagtctgatgttaccagtatactatcagaatgtagcacca
ttgatcttcaggatggctttggatggttacctgagatggttcccaaaaa
gcaaccagagagatgtttgcccaaaggctttggttgctgctttccatgc
tgtagcgtggacaagagaaagcctccctgggtcatttggtggaacctgc
ggaaaacctgctaccaaatagtgaaacacagctggtttgagagctttat
tatctttgtgattctgctgagcagtggggcactgatatttgaagatgtt
caccttgagaaccaacccaaaatccaagaattactaaattgtactgaca
ttatttttacacatatttttatcctggagatggtactaaaatgggtagc
cttcggatttggaaagtatttcaccagtgcctggtgctgccttgatttc
atcattgtgattgtctctgtgaccaccctcattaacttaatggaattga
agtccttccggactctacgagcactgaggcctcttcgtgcgctgtccca
gtttgaaggaatgaaggtggtggtcaatgctctcataggtgccatacct
gccattctgaatgttttgcttgtctgcctcattttctggctcgtatttt
gtattctgggagtatacttcttttctggaaaatttgggaaatgcattaa
tggaacagactcagttataaattataccatcattacaaataaaagtcaa
tgtgaaagtggcaatttctcttggatcaaccagaaagtcaactttgaca
atgtgggaaatgcttacctcgctctgctgcaagtggcaacatttaaggg
ctggatggatattatatatgcagctgttgattccacagagaaagaacaa
cagccagagtttgagagcaattcactcggttacatttacttcgtagtct
ttatcatctttggctcattcttcactctgaatctcttcattggcgttat
cattgacaacttcaaccaacagcagaaaaagttaggtggccaagacatt
tttatgacagaagaacagaagaaatactataatgcaatgaaaaaattag
gatccaaaaaacctcaaaaacccattccacggcctctgaacaaatgtca
aggtctcgtgttcgacatagtcacaagccagatctttgacatcatcatc
ataagtctcattatcctaaacatgattagcatgatggctgaatcataca
accaacccaaagccatgaaatccatccttgaccatctcaactgggtctt
tgtggtcatctttacgttagaatgtctcatcaaaatctttgctttgagg
caatactacttcaccaatggctggaatttatttgactgtgtggtcgtgc
ttctttccattgttagtacaatgatttctaccttggaaaatcaggagca
cattcctttccctccgacgctcttcagaattgtccgcttggctcggatt
ggccgaatcctgaggcttgtccgggctgcacgaggaatcaggactctcc
tctttgctctgatgatgtcgcttccttctctgttcaacattggtcttct
actctttctgattatgtttatctatgccattctgggtatgaactggttt
tccaaagtgaatccagagtctggaatcgatgacatattcaacttcaaga
cttttgccagcagcatgctctgtctcttccagataagcacatcagcagg
ttgggattccctgctcagccccatgctgcgatcaaaagaatcatgtaac
tcttcctcagaaaactgccacctccctggcatagccacatcctactttg
tcagttacattatcatctcctttctcattgttgtcaacatgtacattgc
tgtgattttagagaacttcaatacagccactgaagaaagtgaggaccct
ttgggtgaagatgactttgacatattttatgaagtgtgggaaaagtttg
acccagaagcaacacaatttatcaaatattctgccctttctgactttgc
tgatgccttgcctgagcctttgcgtgtcgcaaagccaaataaatatcaa
tttctagtaatggacttgcccatggtgagtgaagatcgcctccactgca
tggatattcttttcgccttcaccgctagggtactcggtggctctgatgg
cctagatagtatgaaagcaatgatggaagagaagttcatggaagccaat
cctctcaagaagttgtatgaacccatagtcaccaccaccaagagaaagg
aagaggaaagaggtgctgctattattcaaaaggcctttcgaaagtacat
gatgaaggtgaccaagggtgaccaaggtgaccaaaatgacttggaaaac
gggcctcattcaccactccagactctttgcaatggagacttgtctagct
ttggggtggccaagggcaaggtccactgtgactga
H.s. SCN1B
(SEQ ID NO: 16)
Atggggaggctgctggccttagtggtcggcgcggcactggtgtcctcag
cctgcgggggctgcgtggaggtggactcggagaccgaggccgtgtatgg
gatgaccttcaaaattctttgcatctcctgcaagcgccgcagcgagacc
aacgctgagaccttcaccgagtggaccttccgccagaagggcactgagg
agtttgtcaagatcctgcgctatgagaatgaggtgttgcagctggagga
ggatgagcgcttcgagggccgcgtggtgtggaatggcagccggggcacc
aaagacctgcaggatctgtctatcttcatcaccaatgtcacctacaacc
actcgggcgactacgagtgccacgtctaccgcctgctcttcttcgaaaa
ctacgagcacaacaccagcgtcgtcaagaagatccacattgaggtagtg
gacaaagccaacagagacatggcatccatcgtgtctgagatcatgatgt
atgtgctcattgtggtgttgaccatatggctcgtggcagagatgattta
ctgctacaagaagatcgctgccgccacggagactgctgcacaggagaat
gcctcggaatacctggccatcacctctgaaagcaaagagaactgcacgg
gcgtccaggtggccgaatag
H.s. SCN2B
(SEQ ID NO: 17)
Atgcacagagatgcctggctacctcgccctgccttcagcctcacggggc
tcagtctctttttctctttggtgccaccaggacggagcatggaggtcac
agtacctgccaccctcaacgtcctcaatggctctgacgcccgcctgccc
tgcaccttcaactcctgctacacagtgaaccacaaacagttctccctga
actggacttaccaggagtgcaacaactgctctgaggagatgttcctcca
gttccgcatgaagatcattaacctgaagctggagcggtttcaagaccgc
gtggagttctcagggaaccccagcaagtacgatgtgtcggtgatgctga
gaaacgtgcagccggaggatgaggggatttacaactgctacatcatgaa
cccccctgaccgccaccgtggccatggcaagatccatctgcaggtcctc
atggaagagccccctgagcgggactccacggtggccgtgattgtgggtg
cctccgtcgggggcttcctggctgtggtcatcttggtgctgatggtggt
caagtgtgtgaggagaaaaaaagagcagaagctgagcacagatgacctg
aagaccgaggaggagggcaagacggacggtgaaggcaacccggatgatg
gcgccaagtag
H.s. SCN3B
(SEQ ID NO: 18)
Atgcctgccttcaatagattgtttcccctggcttctctcgtgcttatct
actgggtcagtgtctgcttccctgtgtgtgtggaagtgccctcggagac
ggaggccgtgcagggcaaccccatgaagctgcgctgcatctcctgcatg
aagagagaggaggtggaggccaccacggtggtggaatggttctacaggc
ccgagggcggtaaagatttccttatttacgagtatcggaatggccacca
ggaggtggagagcccctttcaggggcgcctgcagtggaatggcagcaag
gacctgcaggacgtgtccatcactgtgctcaacgtcactctgaacgact
ctggcctctacacctgcaatgtgtcccgggagtttgagtttgaggcgca
tcggccctttgtgaagacgacgcggctgatccccctaagagtcaccgag
gaggctggagaggacttcacctctgtggtctcagaaatcatgatgtaca
tccttctggtcttcctcaccttgtggctgctcatcgagatgatatattg
ctacagaaaggtctcaaaagccgaagaggcagcccaagaaaacgcgtct
gactaccttgccatcccatctgagaacaaggagaactctgcggtaccag
tggaggaatag
H.s. SCN4B
(SEQ ID NO: 19)
Atgcccggggctggggacggaggcaaagccccggcgagatggctgggca
ctgggcttttgggcctcttcctgctccccgtaaccctgtcgctggaggt
gtctgtgggaaaggccaccgacatctacgctgtcaatggcacggagatc
ctgctgccctgcaccttctccagctgctttggcttcgaggacctccact
tccggtggacctacaacagcagtgacgcattcaagattctcatagaggg
gactgtgaagaatgagaagtctgaccccaaggtgacgttgaaagacgat
gaccgcatcactctggtaggctctactaaggagaagatgaacaacattt
ccattgtgctgagggacctggagttcagcgacacgggcaaatacacctg
ccatgtgaagaaccccaaggagaataatctccagcaccacgccaccatc
ttcctccaagtcgttgatagactggaagaagtggacaacacagtgacac
tcatcatcctggctgtcgtgggcggggtcatcgggctcctcatcctcat
cctgctgatcaagaaactcatcatcttcatcctgaagaagactcgggag
aagaagaaggagtgtctcgtgagctcctcggggaatgacaacacggaga
acggcttgcctggctccaaggcagaggagaaaccaccttcaaaagtgtg
a
H.s. SCN1A
(SEQ ID NO: 20)
mcqtvlvppgpdsfnffircslaaierriacckaknpkpdkkdddcngp
kpnsdlcagknlpfiygdippcmvscpledldpyyinkktfivlnkgka
ifrfsatsalyiltpfnplrkiaikilvhslfsmlimctiltncvfmtm
snppdwtknveytftgiytfeslikiiargfcledftflrdpwnwldft
vitfayvtefvdlgnvsalrtfrvlralktisvipglktivgaliqsvk
klsdvmiltvfclsvfaliglqlfmgnlrnkciqwpptnasleehsiek
nitvnyngtlinetvfefdwksyiqdsryhyflegfldallcgnssdag
qcpegymcvkagrnpnygytsfdtfswaflslfrlmtqdfwenlyqltl
raagktymiffvlviflgsfylinlilavvamayccqnqatlccacqkc
acfqqmicqlkkqqcaaqqaatataschsrcpsaagrlsdssscaskls
sksakerrnrrkkrkqkeqsggeekdedefqksesedsirrkgfrfsie
gnrltyekryssphqsllsirgslfsprrnsrtslfsfrgrakdvgsen
dfaddehstfednesrrdslfvprrhgerrnsnlsqtsrssrmlavfpa
ngkmhstvdcngvvslvggpsvptspvgqllpegtttetemrkrrsssf
hvsmdfledpsqrqramsiasiltntveeleesrqkcppcwykfsnifl
iwdcspywlkvkhvvnlvvmdpfvdlaiticivlntlfmamehypmtdh
fnnvltvgnlvftgiftaemflkiiamdpyyyfqegwnifdgfivtlsl
velglanveglsvlrsfrllrvfklakswptlnmlikiignsvgalgnl
tlvlaiivfifavvgmqlfgksykdcvckiasdcqlprwhmndffhsfl
ivfrvlcgewietmwdcmevagqamcltvfmmvmvignlvvinlflall
lssfsadnlaatdddnemnnlqiavdrmhkgvayvkrkiyefiqqsfir
kqkildeikplddlnnkkdscmsnhtaeigkdldylkdvngttsgigtg
ssvekyiidesdymsfinnpsltvtvpiavgesdfenlntedfssesdl
eeskeklnesssssegstvdigapveeqpvvepeetlepeacftegcvq
rfkccqinveegrgkqwwnlrrtcfrivehnwfetfivfmillssgala
fediyidqrktiktmleyadkvftyifilemllkwvaygyqtyftnawc
wldflivdvslvsltanalgyselgaikslrfiralrplralsrfegmr
vvvnallgaipsimnyllyclifwlifsimgvnlfagkfyhcintttgd
rfdiedvnnhtdclkliernetarwknvkvnfdnvgfgylsllqvatfk
gwmdimyaavdsrnvelqpkyeeslymylyfvifiifgsfftlnlfigv
iidnfnqqkkkfggqdifmteeqkkyynamkklgskkpqkpiprpgnkf
qgmvfdfvtrqvfdisimiliclnmvtmmvetddqseyvttilsrinlv
fivlftgecvlklislrhyyftigwnifdfvvvilsivgmflaelieky
fvsptlfrvirlarigrilrlikgakgirtllfalmmslpalfniglll
flvmfiyaifgmsnfayvkrevgiddmfnfetfgnsmiclfqittsagw
dgllapilnskppdcdpnkvnpgssvkgdcgnpsvgifffvsyiiisfl
vvvnmyiavilenfsvateesaeplseddfemfyevwekfdpdatqfme
feklsqfaaaleppliflpqpnklqliamdipmvsgdrilicldilfaf
tkrvlgesgemdalriqmeerfmasnpskvsyqpitttlkrkqeevsav
iiqrayrrhllkrtvkqasftynknkikgganllikedmiidrinensi
tektdltmstaacppsydrytkpivekheqegkdekakgk
H.s. SCN2A
(SEQ ID NO: 21)
maqsvlvppgpdsfrfftreslaaieqriaeekakrpkqerkdeddeng
pkpnsdleagkslpfiygdippemvsvpledldpyyinkktfivlnkgk
aisrfsatpalyiltpfnpirklaikilvhslfnmlimcalinevfmtm
snppdwtknveyffigiytfeslikilargfcledftflrdpwnwldft
vitfayvtefvdlgnvsalrtfrvlralktisvipglktivgaliqsvk
klsdvmiltvfclsvfaliglqlfmgnlrnkclqwppdnssfeinitsf
fnnsldgngttfnrtvsifnwdeyiedkshfyflegqndallegnssda
gqcpegyicvkagrnpnygytsfdtfswaflslfrlmtqdfwenlyqlt
lraagktymiffvlviflgsfylinlilavvamayeeqnqatleeaeqk
eaefqqmleqlkkqqeeaqaaaaaasaesrdfsgaggigvfsesssvas
klssksekelknrrlddckqkeqsgeeekndrvrksesedsirrkgfrf
slegsrltyekrfssphqsllsirgslfsprrnsraslfsfrgrakdig
sendfaddehstfedndsrrdslfvphrhgerrhsnvsqasrasrvlpi
lpmngkmhsavdcngvvslvggpstltsagqllpegttteteirkrrss
syhvsmdlledptsrqramsiasiltntmeeleesrqkcppcwykfanm
cliwdcckpwlkvkhlvnlvvmdpfvdlaiticivintlfmamehypmt
eqfssvlsvgnlvftgiftaemflkiiamdpyyyfqegwnifdgfivsl
slmelglanveglsvlrsfrllrvfklakswptlnmlikiignsvgalg
nitivlaiivfifavvgmqlfgksykecyckisndcelprwhmhdffhs
flivfrvlcgewietmwdcmevagqtmcltvfmmvmvignlvvinlfla
lllssfssdnlaatdddnemnnlqiavgrmqkgidfvkrkirefiqkaf
vrkqkaldeikpledlnnkkdscisnhttieigkdlnylkdgngttsgi
gssvekyvvdesdymsfinnpsltvtvpiavgesdfenlnteefssesd
meeskeklnatsssegstvdigapaegeqpevepeeslepeacftedcv
rkfkccqisieegkgklwwnlrktcykivehnwfetfivfmillssgal
afediyieqrktiktmleyadkvftyifilemllkwvaygfqvyftnaw
cwldflivdvslvsltanalgyselgaikslrtlralrplralsrfegm
rvvvnallgaipsimnyllyclifwlifsimgvnlfagkfyhcinyttg
emfdvsvynnyseckaliesnqtarwknvkvnfdnvglgylsllqvatf
kgwmdimyaavdsrnvelqpkyednlymylyfvifiifgsfftlnlfig
viidnfnqqkkkfggqdifmteeqkkyynamkklgskkpqkpiprpank
fqgmvfdfvtkqvfdisimiliclnmvtmmvetddqsqemtnilywinl
vfivlftgecvlklislryyyftigwnifdfvvvilsivgmflaelick
yfvsptlfrvirlarigrilrlikgakgirtllfalmmslpalfnigll
lflvmfiyaifgmsnfayvkrevgiddmfnfetfgnsmiclfqittsag
wdgllapilnsgppdcdpdkdhpgssvkgdcgnpsvgifffvsyiiisf
lvvvnmyiavilenfsvateesaeplseddfemfyevwekfdpdatqfi
efaklsdfadaldpplliakpnkvqliamdlpmvsgdrihcldilfaft
krvlgesgemdalriqmeerfmasnpskvsyepitttlkrkqeevsaii
iqrayrryllkqkvkkvssiykkdkgkecdgtpikedtlidklnenstp
ektdmtpsttsppsydsvtkpekekfekdksekedkgkdireskk
H.s. SCN3A
(SEQ ID NO: 22)
maqallvppgpesfrlftreslaaiekraaeekakkpkkeqdnddenkp
kpnsdleagknlpfiygdippemvsepledldpyyinkktfivmnkgka
ifrfsatsalyiltpinpvrkiaikilvhslfsmlimctiltncvfmtl
snppdwtknveytftgiytfeslikilargfcledftflrdpwnwldfs
vivmayvtefvdlgnvsalrtfrvlralktisvipglktivgaliqsvk
klsdvmiltvfclsvfaliglqlfmgnlinkclqwppsdsafetnttsy
fngtmdsngtfvnytmstfnwkdyigddshfyvldgqkdpllcgngsda
gqcpegyicvkagrnpnygytsfdtfswaflslfrlmtqdywenlyqlt
lraagktymiffvlviflgsfylvnlilavvamayeeqnqatleeaeqk
eaefqqmleqlkkqqeeaqavaaasaasrdfsgigglgellessseask
lssksakewrnrrkkrrqrehlegnnkgerdsfpksesedsvkrssflf
smdgnrltsdkkfcsphqsllsirgslfsprrnsktsifsfrgrakdvg
sendfaddehstfedsesrrdslfvphrhgerrnsnvsqasmssrmvpg
lpangkmhstvdcngvvslvggpsaltsptgqlppegtttetevrkrrl
ssyqismemledssgrqraysiasiltntmeeleesrqkcppcwyrfan
vfliwdccdawlkvkhlvnlivmdpfvdlaiticivintlfmamehypm
teqfssvltvgnlvftgiftaemvlkiiamdpyyyfqegwnifdgiivs
lslmelglsnveglsvlrsfrllrvfklakswptlnmlikiignsvgal
gnitivlaiivfifavvgmqlfgksykecyckinddctlprwhmndfrh
sflivfrvlcgewietmwdcmevagqtmclivfmlvmvignlvvinffl
alllssfssdnlaatdddnemnnlqiavgrrnqkgidyvknkmrecfqk
affrkpkvieihegnkidscmsnntgieiskelnylrdgngttsgvgtg
ssvekyvidendymsfinnpsltvtvpiavgesdfenlnteefssesel
eeskeklnatsssegstvdvvlpregeqactcpeedlkpeacftegcik
kfpfcqvstcegkgkiwwnlrktcysivehnwfetfivfmillssgala
fediyieqrktiktmleyadkvftyifilemllkwvaygfqtyftnawc
wldflivdvslvslvanalgyselgaikslrfiralrplralsrfegmr
vvvnalvgaipsimnyllvclifwlifsimgvnlfagkfyhcvnmttgn
mfdisdvnnlsdcqalgkqarwknvkvnfdnvgagylallqvatfkgwm
dimyaavdsrdvklqpvyeenlymylyfvifilfgsfftlnlfigviid
nfnqqkkkfggqdifmteeqkkyynamkklgskkpqkpiprpankfqgm
vfdfvtrqvfdisimificInmvtmmvetddqgkymtivlsrinlvfiv
lftgefvlkIvslrhyyftigwnifdfvvvilsivgmflaemiekyfvs
ptifrvirlarigrilrlikgakgirtllfalmmslpalfniglllflv
mfiyaifgmsnfayvkkeagiddmfnfetfgnsmiclfqittsagwdgl
lapilnsappdcdpdtihpgssvkgdcgnpsvgifffvsyilisflvvv
nmyiavilenfsvateesaeplseddfemfyevwekfdpdatqfiefsk
lsdfaaaldpplliakpnkvqliamdlpmvsgdrihcldilfaftkrvl
gesgemdalriqmedrfmasnpskvsyepittfikrkqeevsaafiqrn
frcyllkqrlknissnynkeaikgridlpikqdmiidkingnstpektd
gsssttsppsydsvtkpdkekfekdkpekeskgkevrenqk
H.s. SCN4A
(SEQ ID NO: 23)
marpslctlvplgpeclrpftreslaaieqraveeearlqrnkqmeiee
perkprsdleagknlpmiygdpppevigipledldpyysnkktfivink
gkaifrfsatpalyllspfsvvrrgaikvlihalfsmfimitiltncvf
mtmsdpppwsknveyffigiytfeslikilargfcvddftflrdpwnwl
dfsvimmayltefvdlgnisalrtfrvlralktitvipglktivgaliq
svkklsdvmiltvfclsvfalvglqlfmgnlrqkcvrwpppfndtnttw
ysndtwygndtwygnemwygndswyandtwnshaswatndtfdwdayis
degnfyflegsndallcgnssdaghcpegyeciktgrnpnygytsydtf
swaflalfrimtqdywenlfqlfiraagktymifivviifigsfylinl
ilavvamayaeqneatlaedkekeeefqqmlekflkhqeelekakaaqa
leggeadgdpahgkdcngsldtsqgekgaprqsssgdsgisdameelee
ahqkcppwwykcahkvliwnccapwlkfkniihlivmdpfvdlgitici
vintlfmamehypmtehfdnvltvgnlvftgiftaemvlkliamdpyey
fqqgwnifdsfivtlslvelglanvqglsvlisfrllrvfklakswpfi
nmlikfignsvgalgnitivlafivfifavvgmqlfgksykecyckial
denlprwhmhdffhsflivfrilcgewietmwdcmevagqamcitvflm
vmvignlvvlnlflalllssfsadslaasdedgemnnlqiaigriklgi
gfakafllgllhgkilspkdimlslgeadgageageagetapedekkep
peedlkkdnhilnhmgladgppssleldhlnfinnpyltiqvpiasees
dlempteeetdtfsepedskkppqplydgnssvcstadykppeedpeeq
aeenpegeqpeecfteacvqrwpclyvdisqgrgkkwwfirracfkive
hnwfetfivfmillssgalafediyieqrrvirtileyadkvftyifim
emllkwvaygfkvyftnawcwldflivdvsfislvanwlgyselgpiks
lrtlralrplralsrfegmrvvvnallgaipsimnvllvclifwlifsi
mgvnlfagkfyycintttserfdisevnnkseceslmhtgqvrwlnvkv
nydnvglgylsilqvatfkgwmdimyaavdsrekccqpqyevnlymyly
fviffifgsfftlnlfigviidnfnqqlckklggkdifmteeqkkyyna
mldclgskkpqkpiprpqnkiqgmvydlvtkqafditimiliclnmvtm
mvetdnqsqlkvdilyninmifiiiftgeevlkmlalrqyyftvgwnif
dfvvvilsivglalsdliqkyfvspfifrvirlarigrvirlirgakgi
rtllfalmmslpalfniglfiflvmfiysifgmsnfayvkkesgiddmf
nfetfgnsiiclfeittsagwdgllnpilnsgppdcdpnlenpgtsvkg
dcgnpsigicffcsyiiisflivvnmyiaiilenfnvateesseplged
dfemfyetwekfdpdatqfiaysrlsdfvdtlqeplriakpnkiklitl
dlpmvpgdkihcldilfaltkevlgdsgemdalkqtmeekfmaanpskv
syepittfikrkheevcaikiqrayrrhliqrsmkqasymyrhshdgsg
ddapekegllantmskmyghengnssspspeekgeagdagptmglmpis
psdtawppapppgqtvrpgykeslv
H.s. SCN5A
(SEQ ID NO: 24)
manfllprgtssfrrftreslaaiekrmaekqargsttlqesreglpee
eaprpqldlqaskklpdlygnppqeligepledldpfystqktfivink
gktifrfsatnalyvlspfhpirraavkilvhslfnmlimctiltncvf
maqhdpppwtkyveytftaiytfeslvkilargfclhaftfirdpwnwl
dfsviimayttefvdlgnvsalrtfrvlralktisvisglktivgaliq
svkkladvmvitvfclsvfaliglqlfmgnirhkcyrnftaingtngsv
eadglvwesldlylsdpenyllkngtsdvilcgnssdagtcpegyrclk
agenpdhgytsfdsfawaflalfrlmtqdcwerlyqqfirsagkiymif
fmlviflgsfylvnlilavvamayeeqnqatiaeteekekrfqeameml
kkehealtirgvdtvsrsslemsplapvnsherrskrrkrmssgteecg
edrlpksdsedgpramnhlsltrglsrtsmkprssrgsiftfurdlgse
adfaddenstageseshhtsllvpwplutsaqgqpspgtsapghalhgk
knstvdcngvvsllgagdpeatspgshllrpvmlehppdtttpseepgg
pqmltsqapcvdgfeepgarqralsaysyltsaleeleesrhkcppcwn
rlaqryliweccplwmsikqgvklyvmdpftdltitmcivlntlfmale
hynmtsefeemlqvgnlvftgiftaemtfkiialdpyyyfqqgwnifds
iivilslmelglsrmsnlsvlrsifilryfklakswptlntlikiigns
vgalgnitlylaiivfifavvgmqlfgknyselrdsdsgllprwhmmdf
fhafliifrilcgcwictmwdcmcvsgqslcllvfllvmvignlvvlnl
flalllssfsadnitapdedremnnlqlalariqrglrfvkrttwdfcc
gllrqrpqkpaalaaqgqlpsciatpysppppetekvpptrketrfeeg
eqpgqgtpgdpepvcvpiavaesdtddqeedeenslgteeesskqqesq
pvsggpeappdsrtwsqvsatasseaeasasqadwrqqwkaepqapgcg
etpedscsegstadmtntaelleqipdlgqdvkdpedcftegcvrrcpc
cavdttqapgkvwwrlrktcyhivehswfetfiifmillssgalafedi
yleerktikvlleyadkmftyvfylemllkwvaygfkkyftnawcwldf
livdvslvslvantlgfaemgpikslrtlralrplralsrfegmrvvyn
alvgaipsimnvllvclifwlifsimgvnlfagkfgrcinqtegdlpln
ytivnnksqceslnitgelywtkvkvnfdnvgagylallqvatfkgwmd
imyaavdsrgyeeqpqweynlymyiyfvifiifgsfftlnlfigviidn
fnqqldcklggqdifmteeqkkyynamkklgskkpqkpiprpinkyqgf
ifdivtkqafdvtimfliclnmvtmmvetddqspekinilakinllfva
iftgecivklaalrhyyftnswnifdfvvvilsivgtvlsdiiqkyffs
ptlfrvirlarigrilrlirgakgirtllfalmmslpalfniglllflv
mfiysifgmanfayvkweagiddmfnfqtfansmlclfqittsagwdgl
lspilntgppycdptlpnsngsrgdcgspavgilffttyiiisflivvn
myiaiilenfsvateesteplseddfdmfyeiwekfdpeatqfieysvl
sdfadalseplriakpnqislinmdlpmvsgdrihcmdilfaftkrvlg
esgemdalkiqmeekfmaanpskisyepitttlrrkheevsamviqraf
rrhllqrslkhasflfrqqagsglseedaperegliayvmsenfsrplg
ppssssisstsfppsydsvtratsdnlqvrgsdyshsedladfppspdr
dresiv
H.s. SCN7A (occasionally referred to as SCN6A)
(SEQ ID NO: 25)
mlaspepkglvpftkesfelikqhiakthnedheeedlkptpdlevgkk
lpfiygnlsqgmvsepledvdpyyykkkntfivlnknrtifrfnaasil
ctlspfncirrttikvlvhpffqlfilisvlidcvfmsltnlpkwrpvl
entllgiytfeilvklfargvwagsfsflgdpwnwldfsvtvfeviiry
spldfiptlqtartlrilkiiplnqglkslvgvlihclkqligviiltl
ifisifsligmglfingnlkhkcfrwpqenenetllmrtgnpyyirete
nfyylegeryallegnrtdagqcpegyvcvkaginpdqgftnfdsfgwa
lfalfrlmaqdypevlyhqilyasgkvymiffvvvsflfsfymaslflg
ilamayeeekqrvgeiskkiepkfqqtgkelqegnetdeaktiqiemkk
rspistdtsldvledatlrhkeelekskkicplywykfaktfliwncsp
cwlklkefvhriimapftdlfliiciilnvcfltlehypmskqtntlln
ignlvfigiftaemifkiiamhpygyfqvgwnifdsmivfhglielcla
nvagmallrlfrmlrifklgkywptfqilmwslsnswvalkdlvlllft
fiffsaafgmklfgknyeefvchidkdcqlprwhmhdffhsflnyfril
cgewvetlwdcmevagqswcipfylmvilignllvlylflalvssfssc
kdvtaeenneaknlqlavarikkginyvllkilcktqnvpkdtmdhvne
vyvkedisdhtlselsntqdflkdkekssgteknatenesqslipspsy
setypiasgesdienldnkeiqsksgdggskekikqssssecstydiai
seeeemfyggerskhlkngcrrgsslgqisgaskkgkiwqnirktccki
vennwfkcfiglvtllstgtlafediymdqrktikilleyadmiftyif
ilemllkwmaygfkayfsngwyrldfvvvivfclsligktreelkplis
mkflrplrvlsqfermkvvvralikttlptlnvflyclmiwlifsimgv
dlfagrfyecidptsgerfpssevmnksrcesllfnesmlwenakmnfd
nvgngflsllqvatfngwitimnsaidsvavniqphfevniymycyfin
fiifgvflplsmlitviidnfnkhkiklggsnifitvkqrkqyrrlkkl
myedsqrpvprpinklqgfifdvvtsqafnvivmvlicfqaiammidtd
vqslqmsialywinsifvmlytmecilkliafrcfyftiawnifdfmvv
ifsitglclpmtvgsylvppslvqlillsriihmlrlgkgpkvfhnlml
plmlslpallniilliflvmfiyavfgmynfayvkkeagindvsnfetf
gnsmlclfqvaifagwdgmldaifnskwsdcdpdkinpgtqvrgdcgnp
svgifyfvsyiliswliivnmyivvymeflniaskkknktlseddfrkf
fqvwkrfdpdrtqyidssklsdfaaaldpplfmakpnkgqlialdlpma
vgdrihcldillaftkrvmgqdvrmekvvseiesgfflanpfkitcepi
tttlkrkqeaysatiiqrayknyrlrrndkntsdihmidgdrdvhatke
gayfdkakekspiqsqi
H.s. SCN8A
(SEQ ID NO: 26)
maarllappgpdsfkpftpeslanicrriacsklkkppkadgshrcddc
dskpkpnsdleagkslpfiygdipqglvavpledfdpyyltqktfvvin
rgktlfrfsatpalyilspfnlirriaikilihsvfsmiimctiltncv
fmtfsnppdwsknveytftgiytfeslvkiiargfcidgftflrdpwnw
ldfsvimmayitefvnlgnvsalrtfrvlralktisvipglktivgali
qsvkklsdvmiltvfclsvfaliglqlfmgnlrnkcvvwpinfnesyle
ngtkgfdweeyinnktnfytvpgmlepllcgnssdagqcpegyqcmkag
rnpnygytsfdtfswaflalfrlmtqdywenlyqltlraagktymiffy
lvifvgsfylvnlilavvamayeeqnqatlecaeqkeacfkamleqlkk
qqccaqaaamatsagtvsedaiecegeegggsprssseisklssksake
rrnrrkkrkqkelsegeekgdpekvfksesedgmrrkafrlpdnrigrk
fsimilqsllsipgspflsrhnskssifsfrgpgrfrdpgsenefadde
hstveesegrrdslfipirarerrssysgysgysqgsrssrifpslrrs
vkrnstvdengvvsliggpgshiggrllpeatteveikkkgpgsllvsm
dqlasygrkdrinsimsvvtntiveeleesqrkcppcwykfantfliwe
chpywiklkeivnlivmdpfvdlaiticivintlfmamehhpmtpqfeh
vlavgnlvftgiftaemflkliamdpyyyfqegwnifdgfivslslmel
sladveglsvirsifilrvfklakswptlnmlikiignsvgalgnifiv
laiivfifavvgmqlfgksykecvckinqdcelprwhmhdffhsflivf
rvlcgewietmwdcmevagqamclivfmmvmvignlvvinlflaHissf
sadnlaatdddgemnnlqisvirikkgvawtklkvhafmqahfkqread
evkpldelyekkancianhtgadihrngdfqkngngttsgigssvekyi
idedhmsfinnpnitvrvpiavgesdfenlntedvssesdpegskdkld
dtsssegstidikpeveevpveqpeeyldpdacftegcvqrfkccqvni
eeglgkswwilrktcflivehnwfetfiifmillssgalafediyieqr
ktirtileyadkvftyifilemllkwtaygfvkfftnawcwldflivay
slvslianalgyselgaikslrtlralrplralsrfegmrvvvnalvga
ipsimnvllvclifwlifsimgvnlfagkyhycfnetseirfeiedvnn
kteceklmegnnteirwknvkinfdnvgagylallqvatfkgwmdimya
avdsrkpdeqpkyedniymyiyfvifiifgsfftlnlfigviidnfnqq
kkkfggqdifmteeqkkyynamkklgskkpqkpiprpinkiqgivfdfv
tqqafdivimmliclnmvtmmvetdtqskqmenilywinlvfvifftce
cvlkmfalrhyyftigwnifdfvvvilsivgmfladiiekyfvsptlfr
virlarigrilrlikgakgirtllfalmmslpalfniglllflvmfifs
ifgmsnfayvkheagiddmfnfetfgnsmiclfqittsagwdglllpil
nrppdcsldkehpgsgfkgdcgnpsvgiffivsyiiisflivvnmyiai
ilenfsvateesadplseddfetfyeiwekfdpdatqfieyckladfad
alehplrvpkpntieliamdlpmvsgdrihcldilfaftkrvlgdsgel
dilrqqmeerfvasnpskvsyepittfirrkqeevsavvlqrayrghla
rrgfickkttsnklenggthrekkestpstaslpsydsvtkpekekqqr
aeegrrerakrqkevreskc
H.s. SCN9A
(SEQ ID NO: 27)
mamlpppgpqsfvhftkqslalieqriaerkskepkeekkdddeeapkp
ssdleagkqlpfiygdippgmvsepledldpyyadkktfivinkgktif
rfnatpalymlspfsplrrisikilvhslfsmlimctiltncifmtmnn
ppdwtknveytftgiytfeslvkilargfcvgeftflrdpwnwldfvvi
vfayltefvnlgnvsalrtfrvlralktisvipglktivgaliqsvkkl
sdvmiltvfclsvfaliglqlfmgnlkhkcfrnslennetlesimntle
seedfrkyfyylegskdallcgfstdsgqcpegytcvkigrnpdygyts
fdtfswaflalfrlmtqdywenlyqqtlraagktymiffvvviflgsfy
linlilavvamayeeqnqanieeakqkelefqqmldrlkkeqeeaeaia
aaaaeytsirrsrimglsesssetsklssksakermakkknqkklssge
ekgdaeklsksesedsirrksfhlgveghrrahekrlstpnqsplsirg
slfsarrssrtslfsfkgrgrdigsetefaddehsifgdnesrrgslfv
phrpqerrssnisqasrsppmlpvngkmhsavdcngvvslvdgrsalml
pngqllpegttnqihkkrrcssyllsednalndpnlrqramsrasiltn
tveeleesrqkcppwwyrfahkfliwncspywikfkkciyfivmdpfvd
laiticivintlfmamehhpmteefkavlaignlvftgifaaemvlkli
amdpyeyfqvgwnifdslivtlslvelfladveglsvlrsfrllrvfkl
akswptlnmlikiignsvgalgnitivlaiivfifavvgmqlfgksyke
cyckinddctlprwhmndffhsflivfMcgewietmwdcmevagqamcl
ivymmvmvignlvvinlflalllssfssdnitaieedpdannlqiavtr
ikkginyvkqdrefilkafskkpkisreirqaedlntkkenyisnhtla
emskghnflkekdkisgfgssvdkhlmedsdgqsfihnpsltvtvpiap
gesdlenmnaeelssdsdseyskvrinrssssecstvdnplpgegeeae
aepmnsdepeacftdgcvafsccqvniesgkgkiwwnirktcykivehs
wfesfivlmillssgalafediyierldctikiileyadkiftyifile
mllkwiaygyktyftnawcwldflivdvslvtivantlgysdlgpiksl
rtlralrplralsrfegmryvvnaligaipsimmTllvclifwlifsim
gvnlfagkfyecinttdgsrfpasqvpnrsecfalmnvsqnvrwknlkv
nfdnvglgylsllqvatfkgwtiimyaavdsvnvdkqpkyeyslymyiy
fvvfiifgsfftlnlfigviidnfnqqkkklggqdifmteeqkkyynam
kklgskkpqkpiprpgnkiqgcifdlvinqafdisimvliclnmvtmmv
ekegqsqhmtevlywinvvfiilftgecvlklislrhyyftvgwnifdf
vvviisivgmfladlietyfvsptlfrvirlarigrilrlvkgakgirt
llfalmmslpalfniglllflvmfiyaifgmsnfayvkkedgindmfnf
etfgnsmiclfqittsagwdgllapilnskppdcdpkkvhpgssvcgdc
gnpsvgifyfvsyiiisflvvvnmyiavilenfsvateesteplseddf
emfycvwckfdpdatqfiefsklsdfaaaldpplliakpnkvqliamdl
pmvsgdrihcldilfaftkrvlgesgemdslrsqmeerfmsanpskvsy
epittfikrkqedvsatviqrayrrydrqnvknissiyikdgdrdddll
nkkdmafdnvnensspektdatssttsppsydsvtkpdkekyeqdrtek
edkgkdskeskk
H.s. SCN10A
(SEQ ID NO: 28)
mefpigsletnnfrrftpeslveiekqiaakqgtkkarekhreqkdqee
kprpqldlkacnqlpkfygelpaeligepledldpfysthrtfmvinkg
rtisrfsatralwlfspfnlirrtaikvsvhswfslfitvtilvncycm
trtdlpekieyvftviytfealikilargfclneftylrdpwnwldfsv
itlayvgtaidirgisglrtfrviralktysvipglkvivgalihsvkk
ladvtiltifclsvfalvglqlfkgniknkcvkndmavnettnysshrk
pdiyinkrgtsdplicgngsdsghcpdgyiclktsdnpdfnytsfdsfa
waflslfrlmtqdswerlyqqfirtsgkiymiffylviflgsfylynli
lavvtmayeeqnqattdeieakekkfqealemlrkeqevlaalgidtts
lhshngspltsknaserrhrikprvsegstednksprsdpynqrrmsfl
glasgkrrashgsvfhfrspgrdislpegvtddgvfpgdheshrgsfil
gggagqqgplprsplpqpsnpdsrhgedehqppptselapgavdvsafd
agqkktflsaeyldepfraqramsvysiitsvleeleeseqkcppclts
lsqkyliwdccpmwvklkfilfglvtdpfaeltiticivvntifmameh
hgmsptfeamlqignivftifftaemvfkiiafdpyyyfqkkwnifdci
ivtvsllelgvakkgslsvirsfrllrvfklakswptintiikiignsv
galgnitiilaiivfvfalvgkqllgenyrnnrknisaphedwprwhmh
dffhsflivfrilcgewienmwacmevgqksiclilfltvmvlgnlvvi
nlfialllnsfsadnitapeddgevnnlqvalariqvfghrtkqalcsf
fsrscpfpqpkaepelvvklplssskaenhiaantargssgglqaprgp
rdehsdfianptywysvpiaegesdlddleddggedaqsfqqevipkgq
qeqlqqvercgdhltprspgtgtssedlapslgetwkdesvpqvpaegv
ddtsssegstvdcldpeeilrkipeladdleepddcftegcirhcpcck
ldttkspwdvgwqvrktcyrivehswfesfiifmillssgslafedyyl
dqkptvkalleytdrvftfifvfemllkwvaygfkkyftnawcwldfli
vnislisitakileysevapikalrfiralrplralsrfegmrvvvdal
vgaipsimmillvclifwlifsimgvnlfagkfwrcinytdgefslvpl
sivnnksdckiqnstgsffwvnvkvnfdnvamgylallqvatfkgwmdi
myaavdsrevnmqpkwednyymylyfvifiifggfftlnlfygviidnf
nqqkldclggqdifmteeqldcyynamldclgsldcpqkpiprpinkfq
gfvfdivtrqafditimvliclnmitmmvetddqscektkilgkinqff
vayftgccvmkmfatrqyyftngwnvfdfivvvlsiaslifsailkslq
syfspftfrvirlarigrilrliraakgirfilfalmmslpalfnigll
lflvmflysifgmssfphyrweagiddmfnfqtfansmiclfqittsag
wdgllspilntgppycdpnlpnsngtrgdcgspavgiiffttyiiisfl
imvnmyiavilenfnvateesteplseddfdmfyetwekfdpeatqfit
fsalsdfadftsgplripkpnrniliqmdlplvpgdkihcldilfaftk
nvlgesgeldslkanmeekfmatnlskssyepiatftrwkqedisatvi
qkayrsyylhrsmalsntpcvpracccaaslpdegfvaftancncvlpd
ksctasatsfppsycsvtrglsdrvnmrtsssiqncdcatsmeliapgp
H.s. SCN11A
(SEQ ID NO: 29)
mddrcypvifpdernfrpftsdslaaiekriaigkekkkskdqtgevpq
prpqldlkasrklpklygdipreligkpledldpfyrnhktfmvinrkr
tiyrfsakhalfifgpfnsirslairvsvhslfsmfligtviincvfma
tgpaknsnsnntdiaecvftgiyifealikilargfildefsflrdpwn
wldsivigiaivsyipgitikllplrtfrvfralkaisvvsrlkvivga
llrsvkklvnvidtffclsifalvgqqlfmgslnlkcisrdcknisnpc
aydhcfckkenspefkmcgiwmgnsacsiqyeckhtkiripdynytnfd
nfgwsflamfrlmtqdsweklyqqtirttglysvfffivviflgsfyli
nitiavvtmayeeqnknvaaeieakekmfgeaqqllkeekealvamgid
rssltsletsyflpkkrklfgnkkrksfflresgkdqppgsdsdedcqk
kpqlleqtkrlsqnlsldhfdehgdplqrqralsaysiltitmkeqeks
qepclpcgenlaskylvwnccpqwlcvkkvlrtvmtdpftelaiticii
intvflamehhkmeasfekmlnignlvftsifiaemclkiialdpyhyf
rrgwnifdsivallsfadvmncvlqkrswpflrsfrvlrvfklakswpf
intlikiignsvgalgsltvvlvivififsvvgmqlfgrsfnsqkspkl
cnptgptvselrhwhmgdfwhsflvvfrilcgewienmwecmqeanass
slcvivfilitvigklvvinlfialllnsfsneerngnlegearktkvq
laldrfrrafcfvrhtlehfchkwerkqnlpqqkevaggeaaqskdiip
lvmemkrgsetqeelgiltsvpktlgvrhdwtwlaplaeeeddvefsge
dnagritqpepeqqayelhqenkkptsqrvqsveidmfsedephltiqd
prkksdvtsilsecstidlqdgfgwlpemvpkkqperclpkgfgccfpc
csvdkrkppwviwwnlrktcyqivkhswfesfiifvillssgalifedv
hlenqpkiqellnctdiifthifilemvlkwvafgfgkyftsawccldf
iivivsvttlinlmelksfrtlralrplralsqfegmkvvvnaligaip
ailnvllvclifwlvfcilgvyffsgkfgkcingtdsvinytiitnksq
eesgnfswinqkvnfdnvgnaylallqvatfkgwmdiiyaavdstekeq
qpefesnslgyiyfvvfiifgsfftlnlfigviidnfnqqqkklggqdi
fmteeqkkyynamkklgskkpqkpiprpinkcqglvfdivtsqifdiii
islifinmismmaesynqpkamksildhlnwvfvviftleclikifalr
qyyftngwnlfdcvvvllsivstmistlenqehipfpptlfrivrlari
grilfivraargirtllfalmmslpslfnigillflimfiyailgmnwf
skvnpesgiddifnfldfassmlclfqistsagwdsllspmlrskescn
sssenchlpgiatsyfvsyiiisflivvnmyiavilenfntateesedp
lgeddfdifyevwekfdpeatqfikysalsdfadalpeplrvakpnkyq
flvmdlpmvsedrlhcmdilfaftarvlggsdgldsmkammeekfmean
plkklyepivtttkrkeeergaaiiqkafrkymmkvtkgdqgdqndlen
gphsplqtlcngdlssfgvakgkvhcd
H.s. SCN1B
(SEQ ID NO: 30)
Mgrllalvvgaalvssacggcvevdseteavygmtfkilcisckrrset
naetftewtfrqkgteefvkilryenevlqleederfegrvvwngsrgt
kdlqdlsifitnvtynhsgdyechvyrllffenyehntsvvkkihievv
dkanrdmasivseimmyvlivvltiwlvacmiycykkiaaatctaaqcn
ascylaitscskcnctgvqvac
H.s. SCN2B
(SEQ ID NO: 31)
Mhrdawlprpafsltglslffslvppgrsmevtvpatlnvlngsdarlp
ctfnscytvnhkqfslnwtyqecnncseemflqfrmkiinlklerfqdr
vefsgnpskydvsvmlrnvqpedegiyncyimnppdrhrghgkihlqvl
meepperdstvavivgasvggflavvilvlmvvkcvrrkkeqklstddl
kteeegktdgegnpddgak
H.s. SCN3B
(SEQ ID NO: 32)
Mpafnrlfplaslvliywvsvcfpvevevpseteavqgnpmklrciscm
kreeveattvvewfyrpeggkdfliyeyrnghqevespfqgrlqwngsk
dlqdvsitvlnvtlndsglytcnvsrefefeahrpfvkttrliplrvte
eagedftsvvseimmyillvfltlwlliemiycyrkvskaeeaaqenas
dylaipsenkensavpvee
H.s. SCN4B
(SEQ ID NO: 33)
Mpgagdggkaparwlgtgllglfllpvtlslevsvgkatdiyavngtei
llpctfsscfgfedlhfrwtynssdafkiliegtvkneksdpkvtlkdd
dritlvgstkekmnnisivlrdlefsdtgkytchvknpkennlqhhati
flqvvdrleevdntvtliilavvggvigllilillikkliifilkktre
kkkeclvsssgndntenglpgskaeekppskv
Signaling probe 3-(binds target 3)
(SEQ ID NO: 34)
5′-Fam GCGAGAGCGACAAGCAGACCCTATAGAACCTCGC
BHQ1 quench-3′

1. A cell line or cell from the cell line, engineered to stably express a NaV comprising a human alpha 9 subunit, a human beta 1 subunit and a human beta 2 subunit wherein the cell line or cell from the cell line produces a Z′ factor of at least 0.5 in an assay and wherein the expression levels of the NaV subunits do not vary by more than 20% for at least 15 days, 30 day, 45 days, 60 days, 75, days, 100 days, 120 days, or 150 days of continuous culture.

2. The cell line or cell from the cell line of claim 1, wherein the expression levels of the NaV subunits do not vary by more than 10% for at least 15 days, 30 day, 45 days, 60 days, 75, days, 100 days, 120 days, or 150 days of continuous culture.

3. The cell line or cell from the cell line of claim 1 or 2, wherein the cell line or cell from the cell line is grown in the absence of selective pressure.

4.-7. (canceled)

8. The cell line or cell from the cell line of claim 1, wherein the NaV comprises:
a) a NaV subunit that is expressed from an introduced nucleic acid encoding it,
b) a NaV subunit is expressed from an endongenous gene activated by gene activation; or
c) a combination of a) and b).

9. (canceled)

10. The cell line or cell from the cell line of claim 1, wherein cell line or cell from the cell line does not express endogenous NaV prior to engineering.

11. The cell line or cell from the cell line of claim 1, wherein the NaV does not comprise any polypeptide tag.

12.-18. (canceled)

19. A collection of the cell line or cell from the cell line of claim 1, wherein the cells or cell lines in the collection express different forms of NaV.

20. The collection of claim 19, wherein the cell line or cells from the cell lines are matched to share the same physiological property to allow parallel processing.

21. The collection of claim 20, wherein the cell or cells from the cell lines are of the same cell type.

22. The collection of claim 20, wherein the physiological property is growth rate.

23. The collection of claim 20, wherein the physiological property is adherence to a tissue culture surface.

24. The collection of claim 20, wherein the physiological property is Z′ factor.

25. The collection of claim 20, wherein the physiological property is expression level of NaV.

26. A method for identifying a NaV modulator, comprising
a) contacting the cell line or cell from the cell line of claim 1, with a test compound; and
detecting a change in a NaV function in a cell compared to a cell not contacted with the test compound, wherein a change in said function indicates that the test compound is a NaV modulator.

27. The method of claim 26, wherein the test compound is a small molecule, a polypeptide, a peptide, or an antibody or an antigen-binding portion thereof.

28. The method of claim 26, wherein the test compound is in a library of compounds.

29. The method of claim 28, where the library is a small molecule library, a combinatorial library, a peptide library or an antibody library.

30. The method of claim 26, wherein the detecting step is selected from a membrane potential assay, an electrophysiology assay and a binding assay.

31. The method of claim 26, wherein the steps are conducted in a high throughout manner.

32. A modulator identified by the method of claim 26.

33. (canceled)

34. A cell engineered to stably express a NaV at a consistent level over time, the cell made by a method comprising the steps of:
a) providing a plurality of cells that express mRNA(s) encoding the NaV;
b) dispersing the cells individually into individual culture vessels, thereby providing a plurality of separate cell cultures;
c) culturing the cells under a set of desired culture conditions using automated cell culture methods characterized in that the conditions are substantially identical for each of the separate cell cultures, during which culturing the number of cells per separate cell culture is normalized, and wherein the separate cultures are passaged on the same schedule;
d) assaying the separate cell cultures to measure expression of the NaV at least twice; and
e) identifying a separate cell culture that expresses the NaV at a level that does not vary by more than 10% in both assays for at least 15 days, 30 days, 45 days, 60 days, 75 days, 100 days, 120 days, or 150 days of continuous culture and that produces a Z′ factor of at least 0.6, 0.65, 0.70, 0.75, 0.80, or 0.85 in a cell-based assay in response to NaV modulator in both assays, thereby obtaining said cell.

35. A method for identifying a NaV modulator, comprising
a) contacting the cell line or cell from the cell line of claim 19, with a test compound; and
b) detecting a change in a NaV function in a cell compared to a cell not contacted with the test compound, wherein a change in said function indicates that the test compound is a NaV
